(19) United States
(12) Patent Application Publication
(10) Pub. No.: US 2022/0105288 A1
(43) Pub. Date: Apr. 7, 2022

(54) RESPIRATORY DISTRESS MANAGEMENT APPARATUS, SYSTEM AND METHOD

(71) Applicant: ZOLL Medical Corporation, Chelmsford, MA (US)

(72) Inventors: George Beck, Salem, MA (US); Brian P Harvey, Belmont, MA (US); Denise R Eizadkhah, Bridgewater, NJ (US); Dorian LeCroy, Parsippany, NJ (US); Gary A Freeman, Waltham, MA (US)

(21) Appl. No.: 17/449,999

(22) Filed: Oct. 5, 2021

Related U.S. Application Data
(60) Provisional application No. 63/198,234, filed on Oct. 5, 2020.
(60) Provisional application No. 63/168,398, filed on Mar. 31, 2021.

(51) Int. Cl. A61M 16/00; G16H 20/40; G16H 40/67; G16H 40/40

(52) U.S. Cl. CPC A61M 16/024; A61M 16/0051; A61M 16/0003; G16H 20/40; G16H 40/67; A61M 2205/054; A61M 2016/0027; A61M 2016/0033; A61M 2230/40; A61M 2230/20; A61M 2205/505; G16H 40/40

(57) ABSTRACT

Respiratory distress apparatuses, systems and methods are described. An example respiratory distress management device includes a housing, and further has a mechanical ventilation apparatus and a controller within the housing. The controller may include a processor and a memory. The controller may be configured to determine whether, at a particular time, a fault mode condition exists. If a fault mode condition is determined not to exist, then the controller may be configured to enable control of the mechanical ventilation apparatus of the respiratory distress management device by a source in delivering mechanical ventilation to a patient, via signals received by the controller from the source. If a fault mode condition is determined to exist, then the controller may be configured to control the mechanical ventilation apparatus of the respiratory distress management device in delivering mechanical ventilation to the patient.

### PRIORITY APPLICATIONS

This application claims priority to U.S. Provisional Application No. 63/198,234, filed Oct. 5, 2020, titled, “RESPIRATORY DISTRESS MANAGEMENT APPARATUS, SYSTEM, AND METHOD” and U.S. Provisional Application No. 63/168,398, filed Mar. 31, 2021, titled, “RESPIRATORY DISTRESS MANAGEMENT APPARATUS, SYSTEM, AND METHOD”.

### BACKGROUND

Respiratory distress is a common patient complaint that can sometimes signal a medical emergency. By some accounts, respiratory distress is a major complaint in approximately 12% of EMS calls. In an associated emergency response, responders may be posed with a number of complex but critical patient care related challenges.

Responding care providers may face a wide range of response settings, including pre- and out-of-hospital settings, from common indoor and outdoor public and private spaces to out-of-hospital settings that could even include battlefield or mass injury event settings. Other response settings could include ambulance or other transportation contexts, hospital or emergency room arrival, hand-off and transitional contexts, or other in-hospital or emergency room settings. In such settings, the responding care providers must sometimes provide life-saving care, which may include providing mechanical ventilation, for substantial periods of time. The responding care providers in such settings, sometimes with relatively limited training or experience, may be faced with urgently caring for a patient experiencing a medical crisis. Furthermore, they may need to do so using taken or carried equipment, and may need to immediately operationally integrate with other care providers and their devices. The scope or effectiveness of their interventions may be limited based on the equipment that they are able to carry with them to the scene. Still further, in some cases, multiple care providers may be needed at the scene, each with different equipment, and sometimes arriving at different times. This can present further challenges relating to, for example, integration and control of communication, physiological signal collection, data, devices, interventions and overall patient care.

Once at the scene, a responding care provider may need to urgently determine and provide an effective patient intervention, where the selected intervention and the speed of its delivery to the patient can be a matter of life or death. This, in turn, may demand urgent patient related assessment, such as in connection with the nature and extent of particular dysfunction or problems, potentially in a challenging pre-hospital context. The assessment may be made more difficult because the etiology leading to patient respiratory distress may be difficult to determine, and may be associated with underlying causes spanning any of a range of patient systems, such as respiratory, cardiac, endocrine or others. Care providers may be faced with performing or obtaining a patient assessment using available patient history and physiological signals that may not, definitively or alone, point clearly to a particular etiology. Furthermore, care providers may face additional challenges during providing care. These challenges may include, for example, care provider distractions or multitasking, physical instability at the scene, limited or intermittent communication or network availability and associated device integration or isolation issues, and changing or evolving patient conditions or etiologies. Such conditions may demand, during the providing of care, ongoing monitoring and assessment as well as determination and implementation of modified interventions or associated operational parameters.

These and other problems and challenges, upon which the well-being or life of the patient may well depend, face responding emergency care providers in various contexts that may include a patient experiencing RD.

### SUMMARY

One example of an apparatus for treating a patient experiencing respiratory distress includes: a respiratory distress management device, comprising: a housing; a mechanical ventilation apparatus, disposed within the housing, for providing mechanical ventilation to the patient, comprising: a gas flow generator disposed within the housing; and a gas delivery apparatus, disposed at least partially within the housing, coupled with the gas flow generator, comprising a patient circuit extending from the housing and configured to interface with the patient at least in part for delivery of gas to the patient; and a controller, disposed within the housing, the controller comprising a processor and a memory, the controller being configured to: determine whether a fault mode condition exists at a particular time; if the fault mode condition is determined not to exist, enable control of the mechanical ventilation apparatus of the respiratory distress management device by at least one source in delivering mechanical ventilation to the patient, via signals received by the controller from the at least one source; and if the fault mode condition is determined to exist, control the mechanical ventilation apparatus of the respiratory distress management device in delivering the mechanical ventilation to the patient.

In some examples, if the fault mode condition is determined to exist at the particular time, the controller controls the mechanical ventilation apparatus of the respiratory distress management device in delivering the mechanical ventilation to the patient according to previously stored ventilation control parameter values. In some examples, if the fault mode condition is determined to exist at the particular time, the controller controls the mechanical ventilation apparatus of the respiratory distress management device in delivering the mechanical ventilation to the patient according to previously stored last received valid ventilation control parameter values. In some examples, the controller is configured to: determine whether the fault mode condition exists, wherein the fault mode condition is associated with the respiratory distress management device, and wherein the at least one source is separate from the respiratory distress management device.

In some examples, the controller is configured to: determine whether a fault mode condition exists at a particular time, wherein the particular time is a first predetermined time; if the fault mode condition is determined not to exist at the first predetermined time, enable control of the mechanical ventilation apparatus of the respiratory distress management device by at least one source in delivering mechanical ventilation to the patient, via signals received by the controller from the at least one source; and if the fault mode condition is determined to exist at the first predetermined time, control the mechanical ventilation apparatus of the respiratory distress management device in delivering the mechanical ventilation to the patient.

In some examples, the controller is configured to: determine whether a fault mode condition exists at a second predetermined time, wherein the second predetermined time follows the first predetermined time by a predetermined period of time; if the fault mode condition is determined not to exist at the second predetermined time, enable control of the mechanical ventilation apparatus of the respiratory distress management device by at least one source in delivering mechanical ventilation to the patient, via signals received by the controller from the at least one source; and if the fault mode condition is determined to exist at the second predetermined time, control the mechanical ventilation apparatus of the respiratory distress management device in delivering the mechanical ventilation to the patient.

In some examples, the controller is configured such that the predetermined period of time is set prior to use of the respiratory distress management device. In some examples, the controller is configured such that the predetermined period of time is set to one second. In some examples, the controller is configured such that the predetermined period of time is set to between 0.1 second and 1 second. In some examples, the controller is configured such that the predetermined period of time is set to between 1 second and 10 seconds. In some examples, the controller is configured such that the predetermined period of time is set by a user of the respiratory distress management device.

In some examples, the mechanical ventilation apparatus comprises: at least one pressure sensor, disposed within the mechanical ventilation apparatus, configured to sense signals representative of gas flow within the gas delivery apparatus; and wherein the controller is configured to: receive the signals representative of the gas flow; based at least in part on the received signals representative of the gas flow, generate respiratory parameter data corresponding with at least one respiratory parameter of the patient; and transmit the respiratory parameter data to a second device coupled with the respiratory distress management device, the respiratory parameter data being for use in determining a respiratory status of the patient.

In some examples, the controller is configured to transmit the respiratory parameter data to the second device for use by the second device in displaying data related to the respiratory status of the patient on a graphical user interface of a display of the second device. In some examples, the controller is configured to transmit the respiratory parameter data to the second device for use by the second device in displaying user-interactive context sensitive guidance relating to treatment of the patient.

In some examples, the controller is configured to transmit the respiratory parameter data to the second device for use by the second device in displaying user-interactive context sensitive guidance relating to treatment of the patient, the context sensitive guidance comprising a therapy recommendation provided to a user of the second device and relating to a therapy to be delivered to the patient at least in part by the respiratory distress management device upon selection by the user of the therapy recommendation. In some examples, the controller is configured to transmit the respiratory parameter data to the second device for use by the second device in displaying user-interactive context sensitive guidance relating to treatment of the patient, the context sensitive guidance including a therapy recommendation provided to a user of the second device and relating to a therapy to be delivered to the patient at least in part by the respiratory distress management device upon selection by the user of the therapy recommendation, wherein the therapy comprises mechanical ventilation.

In some examples, the controller is configured to generate respiratory parameter data corresponding with at least one respiratory parameter of the patient, wherein the at least one respiratory parameter comprises forced vital capacity (FVC) and forced expiratory volume (FEV).

In some examples, the respiratory distress management device, the at least one source and the second device are different devices and are separate from, but coupled with, the respiratory distress management device. In some examples, the at least one source and the second device are identical, and separate from, but coupled with, the respiratory distress management device. In some examples, the mechanical ventilation apparatus comprises at least one spirometer. In some examples, the at least one pressure sensor is coupled with or part of at least one pneumotachometer disposed within the mechanical ventilation apparatus. In some examples, the respiratory distress management device comprises a bronchodilator. In some examples, the controller determines whether the fault mode condition exists using one or more algorithms stored in the memory of the controller and executed by the processor of the controller. In some examples, the controller is configured to enable the control by the at least one source, wherein the at least one source comprises a portable computing device. In some examples, the controller is configured to enable the control by the at least one source, wherein the at least one source comprises a tablet. In some examples, the controller is configured to enable the control by the at least one source, wherein the at least one source comprises a critical care monitor. In some examples, the controller is configured to enable the control by the at least one source, wherein the at least one source comprises a defibrillator. In some examples, the controller is configured to enable the control by the at least one source, wherein the at least one source comprises one or more aspects of a cloud-based computing system.

In some examples, the controller is configured to enable the control by the at least one source, wherein the control by the at least one source comprises utilizing closed loop ventilation control based on oxygen concentration measurements of the patient's blood, wherein the closed loop ventilation control comprises utilizing at least one oxygen saturation sensor to monitor an oxygen concentration of the patient's blood and adjusting oxygen delivery during the delivery of the mechanical ventilation to the patient to maintain the oxygen concentration of the patient's blood at a desired level or range.

In some examples, the controller is configured to enable control by the at least one source, wherein the at least one source comprises a critical care monitor, and wherein the at least one oxygen saturation sensor is part of a pulse oximeter of the critical care monitor. In some examples, the controller is configured to, in the event that the fault mode condition is determined to exist, switch from enabling the control of the mechanical ventilation by the at least one source to the controller controlling the mechanical ventilation.

In some examples, the fault mode condition indicates that the control by the at least one source would be insufficiently safe. In some examples, the fault mode condition indicates that the control by the at least one source would be insufficiently reliable with respect to receipt of mechanical ventilation control data by the controller of the respiratory distress management system from the at least one source. In some examples, the fault mode condition indicates that the control by the at least one source would be suboptimal, with respect to the treatment of the patient, relative to the control by the controller. In some examples, the fault mode condition relates to one or more unsafe patient physiological parameters. In some examples, the fault mode condition indicates that ventilation control data received by the controller from the at least one source is invalid.

In some examples, the fault mode condition indicates that ventilation control data received by the controller from the at least one source is corrupt. In some examples, the fault mode condition indicates that two or more items of ventilation control data received by the controller from the at least one source are inconsistent with each other. In some examples, the fault mode condition indicates that enabling the control by the at least one source would cause operation of the mechanical ventilation system according to at least one ventilation parameter value that exceeds at least one range. In some examples, the at least one range comprises a range associated with sufficient patient safety. In some examples, the at least one range comprises a flow rate range. In some examples, the at least one range comprises a pressure range. In some examples, the fault mode condition indicates that enabling the control by the at least one source would cause operation of the mechanical ventilation system according to values for at least two parameters, wherein the combination of the values would exceed a range of allowed value combinations for the two parameters. In some examples, the values comprise a value for a flow rate and a value for a pressure. In some examples, the range of allowed value combinations is associated with sufficient patient safety.

In some examples, the respiratory distress management device is configured such that, upon a reset of the controller following a controller processing freeze that occurred during the control by the at least one source, if communication ability exists between the respiratory distress management device and the at least one source, the controller enables the control by the at least one source comprising control in accordance with current ventilation control data from the at least one source. In some examples, the respiratory distress management device is configured such that, upon a reset of the controller following a controller processing freeze that occurred during the control by the at least one source, if communication ability does not exist between the respiratory distress management device and the at least one source, the controller controls the mechanical ventilation in accordance with one or more backup ventilation control parameter values stored in the memory of the controller.

In some examples, the controller is configured to perform cyclic redundancy checks on mechanical ventilation control data received from the at least one source. In some examples, the controller is configured to utilize task prioritization in optimization of controller processing operation. In some examples, the controller is configured to perform processing utilizing data sampling protection. In some examples, the controller is configured to perform processing utilizing one or more histograms in data handling in order to minimize data noise. In some examples, the controller is configured to perform processing utilizing a single thread configuration. In some examples, the controller is configured to perform processing utilizing thread-safe operating system objects. In some examples, the controller is configured to perform processing utilizing a round robin software processing design in order to minimize processing delay. In some examples, the controller is configured to perform token count checks on ventilation control data received from the at least one source.

In some examples, the controller is configured to disable software processing interrupts under one or more conditions. In some examples, the controller is configured to perform processing utilizing prioritization with respect to one or more aspects of communication with the at least one source. In some examples, the controller is configured to perform one or more checks relating to a ventilation control data receipt rate from the at least one source. In some examples, the controller is configured perform processing so as to maintain a minimum portion of unused memory in order to protect against potential data storage overload. In some examples, the controller is configured to perform processing utilizing at least one of an acknowledgement (ACK) or non-acknowledgement (NACK) scheme in ensuring receipt of ventilation control data from the at least one source. In some examples, the controller is configured to perform an automatic reset in the event of a software processing freeze or deadlock condition. In some examples, continuous communication between the respiratory distress management device and the at least one source is utilized in correcting any data receipt errors continuously. In some examples, the controller configured to check that the at least one source is properly configured for communication with the respiratory distress management device.

In some examples, the controller is configured to generate user alarms relating to particular detected conditions. In some examples, the user alarms comprise alarms to provide alerts regarding potentially unsafe ventilation conditions. In some examples, the user alarms comprise alarms to provide alerts regarding potentially unsafe respiratory parameter values of the patient. In some examples, the user alarms comprise alarm indications that are for display to a user of the respiratory distress management system on a graphical user interface that is separate from the respiratory distress management system. In some examples, the user alarms comprise alarms relating to ventilation parameter values. In some examples, the user alarms comprise alarms relating to ventilation parameter values relating to the control by the at least one source. In some examples, the user alarms comprise alarms relating to patient physiological parameters. In some examples, the alarms relating to patient physiological parameters comprise one or more alarms relating to a high breath rate of the patient. In some examples, the alarms relating to patient physiological parameters comprise one or more alarms relating to an incomplete exhalation condition of the patient.

One example of system for treating a patient experiencing respiratory distress includes: a respiratory distress management system, comprising: a mechanical ventilation system for providing mechanical ventilation to the patient, comprising at least one pressure sensor configured to sense signals representative of gas flow within the mechanical ventilation system; and a controller, comprising a processor and a memory, the controller being configured to: determine whether a fault mode condition exists at a particular time; if the fault mode condition is determined not to exist, enable control of the mechanical ventilation system of the respiratory distress management system by at least one source in delivering mechanical ventilation to the patient, via signals received by the controller from the at least one source; and if the fault mode condition is determined to exist, control the mechanical ventilation system of the respiratory distress management system in delivering the mechanical ventilation to the patient.

In some examples, the controller is configured to determine whether the fault mode condition exists, wherein the fault mode condition is associated with the respiratory distress management system, and wherein the at least one source is separate from the respiratory distress management system.

In some examples, the controller is configured to: receive the signals representative of the gas flow; based at least in part on the received signals representative of the gas flow, generate respiratory parameter data corresponding with at least one respiratory parameter of the patient; and transmit the respiratory parameter data to a device coupled with the respiratory distress management system, the respiratory parameter data being for use in determining a respiratory status of the patient.

In some examples, the controller is configured to transmit the respiratory parameter data to the device for use by the device in displaying data related to the respiratory status of the patient on a graphical user interface of a display of the device. In some examples, the controller is configured to transmit the respiratory parameter data to the device for use by the device in displaying user-interactive context sensitive guidance relating to treatment of the patient. In some examples, the controller is configured to transmit the respiratory parameter data to the device for use by the device in displaying user-interactive context sensitive guidance relating to treatment of the patient, the context sensitive guidance comprising a therapy recommendation provided to a user of the device and relating to a therapy to be delivered at least in part by the respiratory distress management device upon selection by the user of the therapy recommendation. In some examples, the controller is configured to transmit the respiratory parameter data to the device for use by the device in displaying user-interactive context sensitive guidance relating to treatment of the patient, the context sensitive guidance including a therapy recommendation provided to a user of the device and relating to a therapy to be delivered at least in part by the respiratory distress management system upon selection by the user of the therapy recommendation, wherein the therapy comprises mechanical ventilation.

In some examples, the respiratory distress management system, the at least one source and the device are separate from each other. In some examples, the at least one source and the device are identical and separate from, but coupled with, the respiratory distress management system. In some examples, the at least one pressure sensor is coupled with or part of at least one pneumotachometer disposed within the mechanical ventilation system. In some examples, the controller determines whether the fault mode condition exists using one or more algorithms stored in the memory of the controller and executed by the processor of the controller. In some examples, the controller is configured to enable the control by the at least one source, wherein the at least one source comprises a portable computing device. In some examples, the controller is configured to enable the control by the at least one source, wherein the at least one source comprises a critical care monitor. In some examples, the controller is configured to enable the control by the at least one source, wherein the at least one source comprises a defibrillator. In some examples, the controller is configured to enable the control by the at least one source, wherein the at least one source comprises one or more aspects of a cloud-based computing system.

In some examples, the controller is configured to enable the control by the at least one source, wherein the control by the at least one source comprises utilizing closed loop ventilation control based on oxygen concentration measurements of the patient's blood, wherein the closed loop ventilation control comprises utilizing at least one oxygen saturation sensor to monitor an oxygen concentration of the patient's blood and adjusting oxygen delivery during the delivery of the mechanical ventilation to the patient to maintain the oxygen concentration of the patient's blood at a desired level or range. In some examples, the controller is configured to enable control by the at least one source, wherein the at least one source comprises a critical care monitor, and wherein the at least one oxygen saturation sensor is part of a pulse oximeter of the critical care monitor. In some examples, the controller is configured to, in the event that the fault mode condition is determined to exist, switch from enabling the control of the mechanical ventilation by the at least one source to the controller controlling the mechanical ventilation. In some examples, the fault mode condition indicates that the control by the at least one source would be insufficiently safe.

In some examples, the fault mode condition indicates that the control by the at least one source would be insufficiently reliable with respect to receipt of mechanical ventilation control data by the controller of the respiratory distress management system from the at least one source. In some examples, the fault mode condition indicates that the control by the at least one source would be suboptimal, with respect to the treatment of the patient, relative to the control by the controller. In some examples, the fault mode condition indicates that ventilation control data received by the controller from the at least one source is invalid. In some examples, the fault mode condition indicates that ventilation control data received by the controller from the at least one source is corrupt. In some examples, the fault mode condition indicates that two or more items of ventilation control data received by the controller from the at least one source are inconsistent with each other.

In some examples, the fault mode condition indicates that enabling the control by the at least one source would cause operation of the mechanical ventilation system according to at least one ventilation parameter value that exceeds at least one range. In some examples, the at least one range comprises a range associated with sufficient patient safety. In some examples, the at least one range comprises a flow rate range. In some examples, the at least one range comprises a pressure range. In some examples, the fault mode condition indicates that enabling the control by the at least one source would cause operation of the mechanical ventilation system according to values for at least two parameters, wherein the combination of the values would exceed a range of allowed value combinations for the two parameters. In some examples, the values comprise a value for a flow rate and a value for a pressure. In some examples, the range of allowed value combinations is associated with sufficient patient safety.

In some examples, the respiratory distress management system is configured such that, upon a reset of the controller following a controller processing freeze that occurred during the control by the at least one source, if communication ability exists between the respiratory distress management system and the at least one source, the controller enables the control by the at least one source comprising control in accordance with current ventilation control data from the at least one source. In some examples, the respiratory distress management system is configured such that, upon a reset of the controller following a controller processing freeze that occurred during the control by the at least one source, if communication ability does not exist between the respiratory distress management system and the at least one source, the controller controls the mechanical ventilation in accordance with one or more backup ventilation control parameter values stored in the memory of the controller. In some examples, the controller is configured to perform cyclic redundancy checks on mechanical ventilation control data received from the at least one source. In some examples, the controller is configured to utilize task prioritization in optimization of controller processing operation.

In some examples, the controller is configured to perform processing utilizing data sampling protection. In some examples, the controller is configured to perform processing utilizing one or more histograms in data handling in order to minimize data noise. In some examples, the controller is configured to perform processing utilizing a single thread configuration. In some examples, the controller is configured to perform processing utilizing thread-safe operating system objects. In some examples, the controller is configured to perform processing utilizing a round robin software processing design in order to minimize processing delay. In some examples, the controller is configured to perform token count checks on ventilation control data received from the at least one source. In some examples, the controller is configured to disable software processing interrupts under one or more conditions. In some examples, the controller is configured to perform processing utilizing prioritization with respect to one or more aspects of communication with the at least one source. In some examples, the controller is configured to perform one or more checks relating to a ventilation control data receipt rate from the at least one source. In some examples, the controller is configured perform processing so as to maintain a minimum portion of unused memory in order to protect against potential data storage overload.

In some examples, the controller is configured to perform processing utilizing at least one of an acknowledgement (ACK) or non-acknowledgement (NACK) scheme in ensuring receipt of ventilation control data from the at least one source. In some examples, the controller is configured to perform an automatic reset in the event of a software processing freeze or deadlock condition. In some examples, wherein continuous communication between the respiratory distress management system and the at least one source is utilized in correcting any data receipt errors continuously. In some examples, the controller configured to check that the at least one source is properly configured for communication with the respiratory distress management system.

In some examples, the controller is configured to generate user alarms relating to particular detected conditions. In some examples, the user alarms comprise alarms to provide alerts regarding potentially unsafe ventilation conditions. In some examples, the user alarms comprise alarms to provide alerts regarding potentially unsafe respiratory parameter values of the patient. In some examples, the user alarms comprise alarm indications that are for display to a user of the respiratory distress management system on a graphical user interface that is separate from the respiratory distress management system. In some examples, the user alarms comprise alarms relating to ventilation parameter values relating to the control by the at least one source. In some examples, the user alarms comprise alarms relating to ventilation parameter values relating to the control by the at least one source. In some examples, the user alarms comprise alarms relating to patient physiological parameters. In some examples, the alarms relating to patient physiological parameters comprise one or more alarms relating to a high breath rate of the patient. In some examples, the alarms relating to patient physiological parameters comprise one or more alarms relating to an incomplete exhalation condition of the patient.

One example of a computer-implemented method for treating a patient experiencing respiratory distress includes a controller, disposed within a respiratory distress management device, the controller comprising a processor and a memory, determining whether, at a particular time, a fault mode condition exists, the fault mode condition being associated with the respiratory distress management device and at least one source that is separate from the respiratory distress management device; if the fault mode condition is determined not to exist, the controller enabling control of a mechanical ventilation system of the respiratory distress management device by the at least one source in delivering mechanical ventilation to the patient, via signals received by the controller from the at least one source; and if the fault mode condition is determined to exist, the controller controlling the mechanical ventilation system of the respiratory distress management device in delivering the mechanical ventilation to the patient.

Some examples include the controller determining whether the fault mode condition exists, wherein the mechanical ventilation system comprises: at least one pressure sensor, disposed within a gas delivery system of the mechanical ventilation system, configured to sense signals representative of gas flow within the gas delivery system; and wherein the controller: receives the signals representative of the gas flow; based at least in part on the received signals representative of the gas flow, generates respiratory parameter data corresponding with at least one respiratory parameter of the patient; and transmits the respiratory parameter data to a second device coupled with the respiratory distress management device, the respiratory parameter data being for use in determining a respiratory status of the patient.

Some examples include the controller transmitting the respiratory parameter data to the second device for use by the second device in displaying data related to the respiratory status of the patient on a graphical user interface of a display of the second device. Some examples include the controller transmitting the respiratory parameter data to the second device for use by the second device in displaying user-interactive context sensitive guidance relating to treatment of the patient. Some examples include the controller transmitting the respiratory parameter data to the second device for use by the second device in displaying user-interactive context sensitive guidance relating to treatment of the patient, the context sensitive guidance comprising a therapy recommendation provided to a user of the second device and relating to a therapy to be delivered at least in part by the respiratory distress management device upon selection by the user of the therapy recommendation. Some examples include the controller transmitting the respiratory parameter data to the second device for use by the second device in displaying user-interactive context sensitive guidance relating to treatment of the patient, the context sensitive guidance including a therapy recommendation provided to a user of the second device and relating to a therapy to be delivered at least in part by the respiratory distress management device upon selection by the user of the therapy recommendation, wherein the therapy comprises mechanical ventilation.

Some examples include the controller determining whether the fault mode condition exists, wherein the respiratory distress management device, the at least one source and the second device are different devices and are separate from, but coupled with, the respiratory distress management device. Some examples include the controller determining whether the fault mode condition exists, wherein the at least one pressure sensor is coupled with or part of at least one pneumotachometer disposed within the mechanical ventilation system. Some examples include the controller determines whether the fault mode condition exists using one or more algorithms stored in the memory of the controller and executed by the processor of the controller.

In some examples, the controller enables the control by the at least one source, wherein the at least one source comprises a portable computing device. In some examples, the controller enables the control by the at least one source, wherein the at least one source comprises a critical care monitor. In some examples, the controller enables the control by the at least one source, wherein the at least one source comprises a defibrillator. In some examples, the controller enables the control by the at least one source, wherein the at least one source comprises one or more aspects of a cloud-based computing system.

In some examples, the controller enables the control by the at least one source, wherein the control by the at least one source comprises utilizing closed loop ventilation control based on oxygen concentration measurements of the patient's blood, wherein the closed loop ventilation control comprises utilizing at least one oxygen saturation sensor to monitor an oxygen concentration of the patient's blood and adjusting oxygen delivery during the delivery of the mechanical ventilation to the patient to maintain the oxygen concentration of the patient's blood at a desired level or range.

In some examples, the controller enables the control by the at least one source, wherein the at least one source comprises a critical care monitor, and wherein the at least one oxygen saturation sensor is part of a pulse oximeter of the critical care monitor. In some examples, the controller, in the event that the fault mode condition is determined to exist, switches from enabling the control of the mechanical ventilation by the at least one source to the controller controlling the mechanical ventilation.

Some examples include the controller determining whether the fault mode condition exists, wherein the fault mode condition indicates that the control by the at least one source would be insufficiently safe. Some examples include the controller determining whether the fault mode condition exists, wherein the fault mode condition indicates that the control by the at least one source would be insufficiently reliable with respect to receipt of mechanical ventilation control data by the controller of the respiratory distress management device from the at least one source. Some examples include the controller determining whether the fault mode condition exists, wherein the fault mode condition indicates that the control by the at least one source would be suboptimal, with respect to the treatment of the patient, relative to the control by the controller. Some examples include the controller determining whether the fault mode condition exists, wherein the fault mode condition indicates that ventilation control data received by the controller from the at least one source is invalid. Some examples include the controller determining whether the fault mode condition exists, wherein the fault mode condition indicates that ventilation control data received by the controller from the at least one source is corrupt. Some examples include the controller determining whether the fault mode condition exists, wherein the fault mode condition indicates that two or more items of ventilation control data received by the controller from the at least one source are inconsistent with each other. Some examples include the controller determining whether the fault mode condition exists, wherein the fault mode condition indicates that enabling the control by the at least one source would cause operation of the mechanical ventilation system according to at least one ventilation parameter value that exceeds at least one range.

Some examples include the controller determining whether the fault mode condition exists, wherein the at least one range comprises a range associated with sufficient patient safety. Some examples include the controller determining whether the fault mode condition exists, wherein the at least one range comprises a flow rate range. Some examples include the controller determining whether the fault mode condition exists, wherein the at least one range comprises a pressure range. Some examples include the controller determining whether the fault mode condition exists, wherein the fault mode condition indicates that enabling the control by the at least one source would cause operation of the mechanical ventilation system according to values for at least two parameters, wherein the combination of the values would exceed a range of allowed value combinations for the two parameters. Some examples include the controller determining whether the fault mode condition exists, wherein the values comprise a value for a flow rate and a value for a pressure. Some examples include the controller determining whether the fault mode condition exists, wherein the range of allowed value combinations is associated with sufficient patient safety.

In some examples, upon a reset of the controller following a controller processing freeze that occurred during the control by the at least one source, if communication ability exists between the respiratory distress management device and the at least one source, the controller enables the control by the at least one source comprising control in accordance with current ventilation control data from the at least one source. In some examples, upon a reset of the controller following a controller processing freeze that occurred during the control by the at least one source, if communication ability does not exist between the respiratory distress management device and the at least one source, the controller controls the mechanical ventilation in accordance with one or more backup ventilation control parameter values stored in the memory of the controller.

In some examples, the controller performs cyclic redundancy checks on mechanical ventilation control data received from the at least one source. In some examples, the controller utilizes task prioritization in optimization of controller processing operation. In some examples, the controller performs processing utilizing data sampling protection. In some examples, the controller performs processing utilizing one or more histograms in data handling in order to minimize data noise. In some examples, the controller performs processing utilizing a single thread configuration. In some examples, the controller performs processing utilizing thread-safe operating system objects. In some examples, the controller performs processing utilizing a round robin software processing design in order to minimize processing delay. In some examples, the controller performs token count checks on ventilation control data received from the at least one source. In some examples, the controller disables software processing interrupts under one or more conditions.

In some examples, the controller performs processing utilizing prioritization with respect to one or more aspects of communication with the at least one source. In some examples, the controller performs one or more checks relating to a ventilation control data receipt rate from the at least one source. In some examples, the controller performs processing so as to maintain a minimum portion of unused memory in order to protect against potential data storage overload. In some examples, the controller performs processing utilizing at least one of an acknowledgement (ACK) or non-acknowledgement (NACK) scheme in ensuring receipt of ventilation control data from the at least one source. In some examples, the controller performs an automatic reset in the event of a software processing freeze or deadlock condition. In some examples, continuous communication between the respiratory distress management device and the at least one source is utilized in correcting any data receipt errors continuously. In some examples, the controller checks that the at least one source is properly configured for communication with the respiratory distress management device.

In some examples, the controller generates user alarms relating to particular detected conditions. In some examples, the controller generates the user alarms, wherein the user alarms comprise alarms to provide alerts regarding potentially unsafe ventilation conditions. In some examples, the controller generates the user alarms, wherein the user alarms comprise alarms to provide alerts regarding potentially unsafe respiratory parameter values of the patient. In some examples, the controller generates the user alarms, wherein at the user alarms comprise alarm indications that are for display to a user of the respiratory distress management system on a graphical user interface that is separate from the respiratory distress management system. In some examples, the controller generates the user alarms, wherein the user alarms comprise alarms relating to ventilation parameter values relating to the control by the at least one source. In some examples, the controller generates the user alarms, wherein the user alarms comprise alarms relating to ventilation parameter values relating to the control by the at least one source. In some examples, the controller generates the user alarms, wherein the user alarms comprise alarms relating to patient physiological parameters. In some examples, the controller generates the user alarms, wherein the alarms relating to patient physiological parameters comprise one or more alarms relating to a high breath rate of the patient. In some examples, the controller generates the user alarms, wherein the alarms relating to patient physiological parameters comprise one or more alarms relating to an incomplete exhalation condition of the patient.

On example of an apparatus for treating a patient experiencing respiratory distress includes: a respiratory distress management device, comprising: a housing; a mechanical ventilation apparatus, disposed within the housing, for providing mechanical ventilation to the patient, comprising: a gas flow generator disposed within the housing; and a gas delivery apparatus, disposed at least partially within the housing, coupled with the gas flow generator, comprising a patient circuit extending from the housing and configured to interface with the patient at least in part for delivery of gas to the patient; at least one pressure sensor, disposed within the mechanical ventilation apparatus, configured to sense signals representative of gas flow within the gas delivery apparatus; and a controller, disposed within the housing, configured to: receive the signals representative of the gas flow; based at least in part on the received signals representative of the gas flow, generate respiratory parameter data corresponding with at least one respiratory parameter of the patient; and transmit the respiratory parameter data to a second device coupled with the respiratory distress management device, the respiratory parameter data being for use in determining a respiratory status of the patient; wherein the controller is configured to be capable of enabling control, by at least one source that is separate from the respiratory distress management apparatus, of the mechanical ventilation apparatus in delivering mechanical ventilation to the patient.

In some examples, the controller is configured such that: the controller determines whether, at a particular time, a fault mode condition exists, the fault mode condition being associated with the respiratory distress management device and the at least one source that is separate from the respiratory distress management device; if the fault mode condition is determined not to exist at the particular time, the controller enables control of the mechanical ventilation apparatus of the respiratory distress management device by the at least one source in delivering mechanical ventilation to the patient, via signals received by the controller from the at least one source; and if the fault mode condition is determined to exist at the particular time, the controller controls the mechanical ventilation apparatus of the respiratory distress management device in delivering the mechanical ventilation to the patient.

In some examples, the controller is configured to enable the control by the at least one source, wherein the control by the at least one source comprises utilizing closed loop ventilation control based on oxygen concentration measurements of the patient's blood, wherein the closed loop ventilation control comprises utilizing at least one oxygen saturation sensor to monitor an oxygen concentration of the patient's blood and adjusting oxygen delivery during the delivery of the mechanical ventilation to the patient to maintain the oxygen concentration of the patient's blood at a desired level or range.

In some examples, the controller is configured to transmit the respiratory parameter data to the second device for use by the second device in displaying data related to the respiratory status of the patient on a graphical user interface of a display of the second device. In some examples, controller is configured to transmit the respiratory parameter data to the second device for use by the second device in displaying user-interactive context sensitive guidance relating to treatment of the patient. In some examples, the controller is configured to transmit the respiratory parameter data to the second device for use by the second device in displaying user-interactive context sensitive guidance relating to treatment of the patient, the context sensitive guidance comprising a therapy recommendation provided to a user of the second device and relating to a therapy to be delivered at least in part by the respiratory distress management device upon selection by the user of the therapy recommendation. In some examples, the controller is configured to transmit the respiratory parameter data to the second device for use by the second device in displaying user-interactive context sensitive guidance relating to treatment of the patient, the context sensitive guidance including a therapy recommendation provided to a user of the second device and relating to a therapy to be delivered at least in part by the respiratory distress management device upon selection by the user of the therapy recommendation, wherein the therapy comprises mechanical ventilation.

In some examples, the controller is configured to generate respiratory parameter data corresponding with at least one respiratory parameter of the patient, wherein the at least one respiratory parameter comprises forced vital capacity (FVC) and forced expiratory volume (FEV). In some examples, the at least one pressure sensor is coupled with or part of at least one pneumotachometer disposed within the mechanical ventilation apparatus. In some examples, the controller is configured to enable the control by the at least one source, wherein the at least one source comprises a device comprising a graphical user interface configured to allow user input in connection with adjustment of one or more ventilation control parameters. In some examples, the respiratory distress management device is configured such that the mechanical ventilation apparatus may be controlled according to signals received by the controller of the respiratory distress management device from outside of the respiratory distress management device. In some examples, the controller is configured to determine whether, at a particular time, the mechanical ventilation apparatus is controlled according to the signals received by the controller from outside of the respiratory distress management device.

In some examples, the controller is configured to determine whether, at the particular time, the mechanical ventilation apparatus is controlled according to the signals received by the controller from outside of the respiratory distress management device from the at least one source. In some examples, the controller is configured to determine whether, at the particular time, the mechanical ventilation apparatus is controlled according to the signals received by the controller of the respiratory distress management device from outside of the respiratory distress management device from the at least one source, wherein the at least one source is a critical care monitor. In some examples, the controller is configured to determine whether, at the particular time, the mechanical ventilation apparatus is controlled according to the signals received by the controller of the respiratory distress management device from outside of the respiratory distress management device from the at least one source, wherein the at least one source is a defibrillator. In some examples, the controller is configured to determine whether, at the particular time, the mechanical ventilation apparatus is controlled according to the signals received by the controller of the respiratory distress management device from outside of the respiratory distress management device from the at least one source, wherein the at least one source is a tablet. In some examples, the controller is configured to determine whether, at the particular time, the mechanical ventilation apparatus is controlled according to the signals received by the controller of the respiratory distress management device from outside of the respiratory distress ventilator unit from the at least one source based at least in part on whether, at the particular time, a communication capability exists between the respiratory distress management device and the controlling source.

In some examples, the controller comprises software, stored in the memory, configured to enable control of the mechanical ventilation apparatus according to the signals received by the controller of the respiratory distress management device from outside of the respiratory distress management device. In some examples, the respiratory distress management device is configured to couple with at least one critical care monitor, and wherein the controller is configured to: receive, from the critical care monitor, signals representative of at least one physiological parameter of the patient; and based at least in part on the received signals, generate the respiratory parameter data corresponding with the at least one respiratory parameter of the patient. In some examples, the controller is configured to receive, from the critical care monitor, the signals representative of the at least one physiological parameter of the patient, comprising receiving the signals representative of at least one physiological parameter of the patient from a defibrillator. In some examples, the controller is configured to receive, from the critical care monitor, the signals representative of the at least one physiological parameter of the patient, comprising receiving the signals obtained at least in part via oximetry. In some examples, the controller is configured to receive, from the critical care monitor, the signals representative of the at least one physiological parameter of the patient, comprising receiving the signals obtained at least in part via capnography. In some examples, the respiratory distress management device is configured to operate in a first mode that does not include delivering mechanical ventilation to the patient, or in a second mode that does include delivering mechanical ventilation to the patient.

In some examples, the gas flow generator comprises a blower. In some examples, the blower, in operation, produces a sound of between 40 and 70 decibels at one meter. In some examples, the gas flow generator comprises a turbine. In some examples, the gas flow generator comprises a compressor. In some examples, the housing of the respiratory distress management device has a volume of between 0.125 and 27 L, such as between 5.0 and 27 L, 1.0 and 5.0 L, between 0.5 and 1.0 L, between 0.15 and 0.5 L, or between 0.125 and 0.15 L. In some examples, the respiratory distress management device has a weight of between 0.2 and 5.0 kg, such as between 2.0 and 5.0 kg, between 1.0 and 2.0 kg, between 0.3 and 1.0 kg, or between 0.2 and 0.3 kg. In some examples, the respiratory distress management device is dimensioned such that none of a length, a height and a width of the respiratory distress management device is greater than 300 mm, greater than 150 mm, or greater than 100 mm or greater than 60 mm.

In some examples, the respiratory distress management device is configured to be portable. In some examples, the respiratory distress management device is configured for use in patient treatment inside a vehicle. In some examples, the respiratory distress management device is configured for use in patient treatment outside of a hospital. In some examples, the controller is configured to generate the respiratory parameter data corresponding with the at least one respiratory parameter of the patient, wherein the at least one respiratory parameter comprises at least one of a respiratory compliance and a respiratory resistance.

In some examples, the respiratory distress management device is configured to deliver non-invasive mechanical ventilation to the patient. In some examples, the respiratory distress management device is configured to deliver non-invasive mechanical ventilation to the patient comprising continuous positive airway Pressure (CPAP) ventilation. In some examples, the respiratory distress management device is configured to deliver non-invasive mechanical ventilation to the patient comprising bilevel ventilation. In some examples, the respiratory distress management device is configured to deliver non-invasive mechanical ventilation to the patient comprising high flow nasal cannula (HFNC) ventilation. In some examples, respiratory distress management device is configured to deliver invasive mechanical ventilation to the patient. In some examples, the respiratory distress management device is configured to deliver invasive mechanical ventilation to the patient comprising assist control (AC) ventilation. In some examples, the respiratory distress management device is configured to deliver invasive mechanical ventilation to the patient comprising synchronized intermittent mandatory ventilation (SIMV). In some examples, the respiratory distress management device is configured to deliver invasive mechanical ventilation to the patient comprising ventilation synchronized with cardiopulmonary resuscitation (CPR) chest compressions being delivered to the patient. In some examples, the respiratory distress management device is coupled with an oxygen source for use in the delivery of the gas to the patient.

In one example, a system for treating a patient experiencing respiratory distress includes: a respiratory distress management system, comprising: a mechanical ventilation system for providing mechanical ventilation to the patient, comprising at least one pressure sensor configured to sense signals representative of gas flow within the mechanical ventilation system; and a controller, comprising a processor and a memory, the controller being configured to: receive the signals representative of the gas flow; based at least in part on the received signals representative of the gas flow, generate respiratory parameter data corresponding with at least one respiratory parameter of the patient; and transmit the respiratory parameter data to a device coupled with the respiratory distress management system, the respiratory parameter data being for use in determining a respiratory status of the patient; wherein the controller is configured to be capable of enabling control, by at least one source that is separate from the respiratory distress management system, of the mechanical ventilation system in delivering mechanical ventilation to the patient.

In some examples, the controller is configured such that: the controller determines whether, at a particular time, a fault mode condition exists, the fault mode condition being associated with the respiratory distress management system and the at least one source that is separate from the respiratory distress management system; if the fault mode condition is determined not to exist at the particular time, the controller enables control of the mechanical ventilation system of the respiratory distress management system by the at least one source in delivering mechanical ventilation to the patient, via signals received by the controller from the at least one source; and if the fault mode condition is determined to exist at the particular time, the controller controls the mechanical ventilation system of the respiratory distress management system in delivering the mechanical ventilation to the patient.

In some examples, the controller is configured to enable the control by the at least one source, wherein the control by the at least one source comprises utilizing closed loop ventilation control based on oxygen concentration measurements of the patient's blood, wherein the closed loop ventilation control comprises utilizing at least one oxygen saturation sensor to monitor an oxygen concentration of the patient's blood and adjusting oxygen delivery during the delivery of the mechanical ventilation to the patient to maintain the oxygen concentration of the patient's blood at a desired level or range.

In some examples, the controller is configured to transmit the respiratory parameter data to the device for use by the device in displaying data related to the respiratory status of the patient on a graphical user interface of a display of the device. In some examples, the controller is configured to transmit the respiratory parameter data to the device for use by the device in displaying user-interactive context sensitive guidance relating to treatment of the patient. In some examples, the controller is configured to transmit the respiratory parameter data to the device for use by the device in displaying user-interactive context sensitive guidance relating to treatment of the patient, the context sensitive guidance comprising a therapy recommendation provided to a user of the device and relating to a therapy to be delivered at least in part by the respiratory distress management device upon selection by the user of the therapy recommendation. In some examples, the controller is configured to transmit the respiratory parameter data to the device for use by the device in displaying user-interactive context sensitive guidance relating to treatment of the patient, the context sensitive guidance including a therapy recommendation provided to a user of the device and relating to a therapy to be delivered at least in part by the respiratory distress management system upon selection by the user of the therapy recommendation, wherein the therapy comprises mechanical ventilation.

In some examples, the at least one pressure sensor is coupled with or part of at least one pneumotachometer disposed within a gas delivery system of the mechanical ventilation system. In some examples, the controller is configured to enable the control by the at least one source, wherein the at least one source comprises a device comprising a graphical user interface configured to allow user input in connection with adjustment of one or more ventilation control parameters. In some examples, the respiratory distress management system is configured such that the mechanical ventilation system may be controlled according to signals received by the controller of the respiratory distress management system from outside of the respiratory distress management system. In some examples, the controller is configured to determine whether, at a particular time, the mechanical ventilation system is controlled according to the signals received by the controller from outside of the respiratory distress management system. In some examples, the controller is configured to determine whether, at the particular time, the mechanical ventilation system is controlled according to the signals received by the controller from outside of the respiratory distress management system from the at least one source. In some examples, the controller is configured to determine whether, at the particular time, the mechanical ventilation system is controlled according to the signals received by the controller of the respiratory distress management system from outside of the respiratory distress management system from the at least one source, wherein the at least one source is a critical care monitor.

In some examples, the controller is configured to determine whether, at the particular time, the mechanical ventilation system is controlled according to the signals received by the controller of the respiratory distress management system from outside of the respiratory distress management system from the at least one source, wherein the at least one source is a defibrillator. In some examples, the controller is configured to determine whether, at the particular time, the mechanical ventilation system is controlled according to the signals received by the controller of the respiratory distress management system from outside of the respiratory distress management system from the at least one source, wherein the at least one source is a tablet. In some examples, the controller is configured to determine whether, at the particular time, the mechanical ventilation system is controlled according to the signals received by the controller of the respiratory distress management system from outside of the respiratory distress management system from the at least one source based at least in part on whether, at the particular time, a communication capability exists between the respiratory distress management system and the at least one source.

In some examples, the controller comprises software, stored in the memory, configured to enable control of the mechanical ventilation system according to the signals received by the controller of the respiratory distress management system from outside of the respiratory distress management system. In some examples, the respiratory distress management system is configured to couple with at least one critical care monitor, and wherein the controller is configured to: receive, from the critical care monitor, signals representative of at least one physiological parameter of the patient; and based at least in part on the received signals, generate the respiratory parameter data corresponding with the at least one respiratory parameter of the patient. In some examples, receiving, from the critical care monitor, the signals representative of the at least one physiological parameter of the patient comprises receiving the signals representative of at least one physiological parameter of the patient from a defibrillator. In some examples, receiving, from the critical care monitor, the signals representative of the at least one physiological parameter of the patient comprises receiving the signals obtained at least in part via oximetry. In some examples, receiving, from the critical care monitor, the signals representative of the at least one physiological parameter of the patient comprises receiving the signals obtained at least in part via capnography. In some examples, the respiratory distress management system is configured to operate in a first mode that does not include delivering mechanical ventilation to the patient, or in a second mode that does include delivering mechanical ventilation to the patient.

In some examples, the gas flow generator comprises a blower. In some examples, the blower, in operation, produces a sound of between 40 and 70 decibels at one meter. In some examples, the gas flow generator comprises a turbine. In some examples, the gas flow generator comprises a compressor. In some examples, the respiratory distress management system has a volume of between 0.125 and 27 L, such as between 5.0 and 27 L, 1.0 and 5.0 L, between 0.5 and 1.0 L, between 0.15 and 0.5 L, or between 0.125 and 0.15 L. In some examples, the respiratory distress management system has a weight of between 0.2 and 5.0 kg, such as between 2.0 and 5.0 kg, between 1.0 and 2.0 kg, between 0.3 and 1.0 kg, or between 0.2 and 0.3 kg.

In some examples, the respiratory distress management system is configured to be portable.

In some examples, the respiratory distress management system is configured for use in patient treatment inside a vehicle. In some examples, the respiratory distress management system is configured for use in patient treatment outside of a hospital. In some examples, the controller is configured to generate the respiratory parameter data corresponding with the at least one respiratory parameter of the patient, wherein the at least one respiratory parameter comprises at least one of a respiratory compliance and a respiratory resistance.

In some examples, the respiratory distress management system is configured to deliver non-invasive mechanical ventilation to the patient. In some examples, the respiratory distress management system is configured to deliver non-invasive mechanical ventilation to the patient comprising continuous positive airway Pressure (CPAP) ventilation. In some examples, the respiratory distress management system is configured to deliver non-invasive mechanical ventilation to the patient comprising bilevel ventilation. In some examples, the respiratory distress management system is configured to deliver non-invasive mechanical ventilation to the patient comprising high flow nasal cannula (HFNC) ventilation. In some examples, respiratory distress management system is configured to deliver invasive mechanical ventilation to the patient. In some examples, the respiratory distress management system is configured to deliver invasive mechanical ventilation to the patient comprising assist control (AC) ventilation. In some examples, the respiratory distress management system is configured to deliver invasive mechanical ventilation to the patient comprising synchronized intermittent mandatory ventilation (SIMV). In some examples, the respiratory distress management system is configured to deliver invasive mechanical ventilation to the patient comprising ventilation synchronized with cardiopulmonary resuscitation (CPR) chest compressions being delivered to the patient. In some examples, the respiratory distress management system is coupled with an oxygen source for use in the delivery of the gas to the patient.

In one examples, a computer-implemented method for treating a patient experiencing respiratory distress includes: a controller, disposed within a respiratory distress management device comprising a mechanical ventilation system for providing mechanical ventilation to the patient and comprising at least one pressure sensor configured to sense signals representative of gas flow within the mechanical ventilation system, the controller comprising a processor and a memory: receiving the signals representative of the gas flow; based at least in part on the received signals representative of the gas flow, generating respiratory parameter data corresponding with at least one respiratory parameter of the patient; and transmitting the respiratory parameter data to a second device coupled with the respiratory distress management device, the respiratory parameter data being for use in determining a respiratory status of the patient; wherein the controller is capable of enabling control, by at least one source that is separate from the respiratory distress management device, of the mechanical ventilation system in delivering mechanical ventilation to the patient.

In some examples, the controller enables the control by the at least one source, wherein the control by the at least one source comprises utilizing closed loop ventilation control based on oxygen concentration measurements of the patient's blood, wherein the closed loop ventilation control comprises utilizing at least one oxygen saturation sensor to monitor an oxygen concentration of the patient's blood and adjusting oxygen delivery during the delivery of the mechanical ventilation to the patient to maintain the oxygen concentration of the patient's blood at a desired level or range.

Some examples include the controller determining whether, at a particular time, a fault mode condition exists, the fault mode condition being associated with the respiratory distress management system and the at least one source that is separate from the respiratory distress management system; if the fault mode condition is determined not to exist at the particular time, the controller enabling control of the mechanical ventilation system of the respiratory distress management system by the at least one source in delivering mechanical ventilation to the patient, via signals received by the controller from the at least one source; and if the fault mode condition is determined to exist at the particular time, the controller controlling the mechanical ventilation system of the respiratory distress management system in delivering the mechanical ventilation to the patient.

Some examples include the controller transmitting the respiratory parameter data to the second device for use by the second device in displaying data related to the respiratory status of the patient on a graphical user interface of a display of the second device. Some examples include the controller transmitting the respiratory parameter data to the second device for use by the second device in displaying user-interactive context sensitive guidance relating to treatment of the patient. Some examples include the controller transmitting the respiratory parameter data to the second device for use by the second device in displaying user-interactive context sensitive guidance relating to treatment of the patient, the context sensitive guidance comprising a therapy recommendation provided to a user of the second device and relating to a therapy to be delivered at least in part by the respiratory distress management device upon selection by the user of the therapy recommendation. Some examples include the controller transmitting the respiratory parameter data to the second device for use by the second device in displaying user-interactive context sensitive guidance relating to treatment of the patient, the context sensitive guidance including a therapy recommendation provided to a user of the second device and relating to a therapy to be delivered at least in part by the respiratory distress management device upon selection by the user of the therapy recommendation, wherein the therapy comprises mechanical ventilation.

Some examples include the controller receiving the signals representative of the gas flow, wherein the at least one pressure sensor is coupled with or part of at least one pneumotachometer disposed within a gas delivery apparatus of the mechanical ventilation system. Some examples include the controller allowing control of the mechanical ventilation system of the respiratory distress management device to deliver ventilation to the patient according to signals received by the respiratory distress management device from the at least one source that is separate from the respiratory distress management device. Some examples include determining, at a particular time, if a communication ability exists, between the respiratory distress management device and the at least one source that is separate from the respiratory distress management device, to allow control of the mechanical ventilation system by the at least one source; if the communication ability is determined to exist at the particular time, the controller allowing the control of the mechanical ventilation system by the at least one source; and if the communication ability is determined not to exist at the particular time, the controller controlling the mechanical ventilation system.

Some examples include, if the communication ability is determined to exist at the particular time, the controller allowing the control of the mechanical ventilation system by the at least one source, wherein the control by the at least one source comprises utilization of one or more algorithms stored at least in part on the at least one source. Some examples include, if the communication ability is determined to exist at the particular time, the controller allowing the control of the mechanical ventilation system by the at least one source, the control by the at least one source being without user input. Some examples include if the communication ability is determined not to exist at the particular time, the controller controlling the mechanical ventilation system using one or more algorithms stored at least in part on the controller. Some examples include if the communication ability is determined not to exist at the particular time, the controller controlling the mechanical ventilation system without user input. Some examples include the controller, if the communication ability is determined to exist at the particular time, allowing the control of the mechanical ventilation system by the at least one source, wherein the at least one source comprises a portable computing device.

Some examples include, if the communication ability is determined to exist at the particular time, the controller allowing the control of the mechanical ventilation system by the at least one source, wherein the at least one source comprises a tablet. Some examples include the controller, if the communication ability is determined to exist at the particular time, allowing the control of the mechanical ventilation system by the at least one source, wherein the at least one source comprises a critical care monitor. Some examples include the controller, if the communication ability is determined to exist at the particular time, allowing the control of the mechanical ventilation system by the at least one source, wherein the at least one source comprises a defibrillator.

In some examples, the respiratory distress management device is coupled with a critical care monitor, wherein the controller generates the respiratory parameter data based at least in part on signals received from the critical care monitor.

In one example, an apparatus for treating a patient experiencing respiratory distress include: a respiratory distress management device, comprising: a housing; a mechanical ventilation apparatus, disposed within the housing, for providing mechanical ventilation to the patient, comprising: a gas flow generator disposed within the housing; and a gas delivery apparatus, disposed at least partially within the housing, coupled with the gas flow generator, comprising a patient circuit extending from the housing and configured to interface with the patient at least in part for delivery of gas to the patient; and a controller, disposed within the housing, the controller comprising a processor and a memory, the controller being configured to: determine whether, at a particular time, a communication ability exists, between the respiratory distress management device and at least one source that is separate from the respiratory distress management device, to allow control, by the at least one source, of the gas delivery apparatus of the mechanical ventilation apparatus in delivering mechanical ventilation to the patient; if the communication ability is determined to exist, enable the control, by the at least one source, of the gas delivery apparatus of the mechanical ventilation apparatus in delivering mechanical ventilation to the patient; if the communication ability is determined not to exist, control the gas delivery apparatus of the mechanical ventilation apparatus in delivering mechanical ventilation to the patient.

In some examples, allowing the control by the at least one source comprises allowing closed loop ventilation control based on oxygen concentration measurements of the patient's blood, wherein the closed loop ventilation control comprises: utilizing at least one oxygen saturation sensor, coupled with the patient, to monitor an oxygen concentration of the patient's blood, and adjusting oxygen delivery during the delivering of the mechanical ventilation to the patient to maintain the oxygen concentration of the patient's blood at a desired level or range.

In some examples, the mechanical ventilation apparatus comprises: at least one pressure sensor, disposed within the mechanical ventilation apparatus of the mechanical ventilation apparatus, configured to sense signals representative of gas flow within the gas delivery apparatus; and wherein the controller is configured to: receive the signals representative of the gas flow; based at least in part on the received signals representative of the gas flow, generate respiratory parameter data corresponding with at least one respiratory parameter of the patient; and transmit the respiratory parameter data to a second device coupled with the respiratory distress management device, the respiratory parameter data being for use in determining a respiratory status of the patient.

In some examples, the controller is configured to transmit the respiratory parameter data to the second device for use by the second device in displaying data related to the respiratory status of the patient on a graphical user interface of a display of the second device. In some examples, the controller is configured to transmit the respiratory parameter data to the second device for use by the second device in displaying user-interactive context sensitive guidance relating to treatment of the patient. In some examples, the controller is configured to transmit the respiratory parameter data to the second device for use by the second device in displaying user-interactive context sensitive guidance relating to treatment of the patient, the context sensitive guidance comprising a therapy recommendation provided to a user of the second device and relating to a therapy to be delivered at least in part by the respiratory distress management device upon selection by the user of the therapy recommendation. In some examples, the controller is configured to transmit the respiratory parameter data to the second device for use by the second device in displaying user-interactive context sensitive guidance relating to treatment of the patient, the context sensitive guidance including a therapy recommendation provided to a user of the second device and relating to a therapy to be delivered at least in part by the respiratory distress management device upon selection by the user of the therapy recommendation, wherein the therapy comprises mechanical ventilation.

In some examples, the controller is configured to generate respiratory parameter data corresponding with at least one respiratory parameter of the patient, wherein the at least one respiratory parameter comprises a forced vital capacity (FVC) parameter and a forced expiratory volume (FEV) parameter. In some examples, the respiratory distress management device, the at least one source and the second device are different devices and are separate from, but coupled with, each other. In some examples, the mechanical ventilation apparatus comprises at least one spirometer. In some examples, the at least one pressure sensor is coupled with or part of at least one pneumotachometer disposed within the mechanical ventilation apparatus.

In one example, a system for treating a patient experiencing respiratory distress, the system includes: a respiratory distress management system, comprising: a mechanical ventilation system for providing mechanical ventilation to the patient; and a controller, comprising a processor and a memory, the controller being configured to: determine whether, at a particular time, a communication ability exists, between the respiratory distress management device and at least one source that is separate from the respiratory distress management device, to allow control, by the at least one source, of the mechanical ventilation apparatus in delivering mechanical ventilation to the patient; if the communication ability is determined to exist at the particular time, enable the control, by the at least one source, of the mechanical ventilation apparatus in delivering mechanical ventilation to the patient; if the communication ability is determined not to exist at the particular time, control the gas delivery apparatus of the mechanical ventilation apparatus in delivering mechanical ventilation to the patient; wherein allowing the control by the at least one source comprises allowing closed loop ventilation control based on oxygen concentration measurements of the patient's blood, wherein the closed loop ventilation control comprises: utilizing at least one oxygen saturation sensor, coupled with the patient, to monitor an oxygen concentration of the patient's blood, and adjusting oxygen delivery during the delivering of the mechanical ventilation to the patient to maintain the oxygen concentration of the patient's blood at a desired level or range.

In some examples, the mechanical ventilation system comprises: at least one pressure sensor, disposed within a gas delivery system of the mechanical ventilation system, configured to sense signals representative of gas flow within the gas delivery system; and wherein the controller is configured to: receive the signals representative of the gas flow; based at least in part on the received signals representative of the gas flow, generate respiratory parameter data corresponding with at least one respiratory parameter of the patient; and transmit the respiratory parameter data to a device coupled with the respiratory distress management system, the respiratory parameter data being for use in determining a respiratory status of the patient.

In some examples, the controller is configured to transmit the respiratory parameter data to the device for use by the device in displaying data related to the respiratory status of the patient on a graphical user interface of a display of the device. In some examples, the controller is configured to transmit the respiratory parameter data to the device for use by the device in displaying user-interactive context sensitive guidance relating to treatment of the patient. In some examples, the controller is configured to transmit the respiratory parameter data to the device for use by the device in displaying user-interactive context sensitive guidance relating to treatment of the patient, the context sensitive guidance comprising a therapy recommendation provided to a user of the device and relating to a therapy to be delivered at least in part by the respiratory distress management device upon selection by the user of the therapy recommendation. In some examples, the controller is configured to transmit the respiratory parameter data to the device for use by the device in displaying user-interactive context sensitive guidance relating to treatment of the patient, the context sensitive guidance including a therapy recommendation provided to a user of the device and relating to a therapy to be delivered at least in part by the respiratory distress management system upon selection by the user of the therapy recommendation, wherein the therapy comprises mechanical ventilation.

In some examples, the respiratory distress management system, the at least one source and the device are different devices and are separate from, but coupled with, each other. In some examples, the at least one source and the device are identical and separate from, but coupled with, the respiratory distress management system. In some examples, the at least one pressure sensor is coupled with or part of at least one pneumotachometer disposed within the mechanical ventilation system.

In one example, a computer-implemented method for treating a patient experiencing respiratory distress includes: a controller, disposed within a respiratory distress management device comprising a mechanical ventilation system for providing mechanical ventilation to the patient, the controller comprising a processor and a memory, determining, at a particular time, whether a communication ability exists, between the respiratory distress management device and at least one source that is separate from the respiratory distress management device, to allow control, by the at least one source, of the mechanical ventilation apparatus in delivering mechanical ventilation to the patient; if the communication ability is determined to exist at the particular time, the controller allowing the control, by the at least one source, of the mechanical ventilation apparatus in delivering mechanical ventilation to the patient; if the communication ability is determined not to exist at the particular time, the controller controls the gas delivery apparatus of the mechanical ventilation apparatus in delivering mechanical ventilation to the patient; wherein allowing the control by the at least one source comprises allowing closed loop ventilation control based on oxygen concentration measurements of the patient's blood, wherein the closed loop ventilation control comprises: utilizing at least one oxygen saturation sensor, coupled with the patient, to monitor an oxygen concentration of the patient's blood, and adjusting oxygen delivery during the delivering of the mechanical ventilation to the patient to maintain the oxygen concentration of the patient's blood at a desired level or range.

In some examples, the mechanical ventilation system comprises: at least one pressure sensor, disposed within a gas delivery system of the mechanical ventilation system, configured to sense first signals representative of gas flow within the gas delivery system; and comprising the controller: receiving the first signals representative of the gas flow; based at least in part on the received first signals representative of the gas flow, generating respiratory parameter data corresponding with at least one respiratory parameter of the patient; and transmitting the respiratory parameter data to a second device coupled with the respiratory distress management device, the respiratory parameter data being for use in determining a respiratory status of the patient. Some examples include the controller transmitting the respiratory parameter data to the second device for use by the second device in displaying data related to the respiratory status of the patient on a graphical user interface of a display of the second device. Some examples include the controller transmitting the respiratory parameter data to the second device for use by the second device in displaying user-interactive context sensitive guidance relating to treatment of the patient.

Some examples include the controller transmitting the respiratory parameter data to the second device for use by the second device in displaying user-interactive context sensitive guidance relating to treatment of the patient, the context sensitive guidance comprising a therapy recommendation provided to a user of the second device and relating to a therapy to be delivered at least in part by the respiratory distress management device upon selection by the user of the therapy recommendation. Some examples include the controller transmitting the respiratory parameter data to the second device for use by the second device in displaying user-interactive context sensitive guidance relating to treatment of the patient, the context sensitive guidance including a therapy recommendation provided to a user of the second device and relating to a therapy to be delivered at least in part by the respiratory distress management device upon selection by the user of the therapy recommendation, wherein the therapy comprises mechanical ventilation. Some examples include the controller allowing the control by the at least one source, wherein the respiratory distress management system, the at least one source and the device are different devices and are separate from, but coupled with, each other.

Some examples include the controller receiving the signals representative of the gas flow, wherein the at least one pressure sensor is coupled with or part of at least one pneumotachometer disposed within the mechanical ventilation system. Some examples include the controller: receiving, from a critical care monitoring device, second signals representative of at least one physiological parameter of the patient; and based at least in part on the received first signals and the received second signals, generating the respiratory parameter data corresponding with the at least one respiratory parameter of the patient.

Some examples include the controller receiving the second signals from the critical care monitor, wherein the critical care monitor is a defibrillator. Some examples include the controller receiving the second signals representative of the at least one physiological parameter of the patient, wherein the at least one physiological parameter comprises a blood oxygen concentration parameter. Some examples include the controller receiving the second signals representative of the at least one physiological parameter of the patient, wherein the at least one physiological parameter is obtained using oximetry. Some examples include the controller receiving the second signals representative of the at least one physiological parameter of the patient, wherein the at least one physiological parameter is obtained using capnography.

Some examples include the controller transmitting the respiratory parameter data to the second device, the respiratory parameter data being for use in determining a respiratory status of the patient, wherein the respiratory status is for display on a graphical user interface of the second device. Some examples include the controller transmitting the respiratory parameter data to the second device, wherein the second device comprises a critical care monitor. Some examples include the controller transmitting the respiratory parameter data to the second device, wherein the second device comprises a tablet. Some examples include the controller transmitting the respiratory parameter data to the second device, wherein the second device comprises an aspect of a cloud-based computing system.

### BRIEF DESCRIPTION OF THE DRAWINGS

Various aspects of embodiments of the present disclosure are discussed below with reference to the accompanying figures, which are not intended to be drawn to scale. The figures are included for illustrative purposes and a further understanding of the various aspects and examples. The figures are incorporated in and constitute a part of this specification, but are not intended to limit the scope of the disclosure. In the figures, identical or nearly identical components that are illustrated in various figures may be represented by like numerals. For purposes of clarity, not every component may be labeled in every figure.

FIG. 1 illustrates an example emergency care environment including a respiratory distress ventilator, a CCM and a tablet.

FIG. 2 illustrates an example emergency care environment including a respiratory distress ventilator, a CCM, a tablet and an offsite monitor.

FIG. 3 illustrates an example emergency care environment including a respiratory distress ventilator and a tablet.

FIG. 4 illustrates an example emergency care environment including a respiratory distress ventilator, a tablet and an offsite monitor.

FIG. 5 illustrates an example emergency care environment including a respiratory distress ventilator and a CCM.

FIG. 6 illustrates an example emergency care environment including a respiratory distress ventilator, a CCM and an offsite monitor.

FIG. 7 illustrates an example emergency care environment including a respiratory distress ventilator, a CCM/defibrillator and a tablet.

FIG. 8 illustrates an example modular system including a respiratory distress ventilator, a CCM/defibrillator and a tablet.

FIG. 9 illustrates an example environment for providing and monitoring patient treatment with multiple devices within a medical treatment system.

FIG. 10 illustrates an example respiratory distress ventilator.

FIG. 11 illustrates an example emergency care setting including a respiratory distress ventilator that can be remote controlled.

FIG. 12 illustrates an example of types of signaling in connection with a ventilation control director (VCD) utilizing fault mode condition analysis.

FIG. 13 illustrates an example of a method relating to fault mode condition analysis implemented by a VCD.

FIG. 14 illustrates an example of an emergency care environment including a VCD of a respiratory distress ventilator controller allowing control of respiratory distress ventilator mechanical ventilation by a remote control source.

FIG. 15 illustrates an example of an emergency care environment including a VCD of a respiratory distress ventilator controller controlling respiratory distress ventilator mechanical ventilation due to detection of a fault mode condition.

FIG. 16 illustrates an example of an emergency care environment including a VCD of a respiratory distress ventilator controller controlling respiratory distress ventilator mechanical ventilation due to detection of a no communication fault mode condition.

FIG. 17 illustrates an example of an emergency care environment including remote control of the respiratory distress ventilator by an off-site source.

FIG. 17A illustrates an example of a non-hospital emergency care environment including a respiratory distress ventilator and multiple other devices.

FIG. 17B illustrates an example of a portion of the non-hospital emergency care environment depicted in FIG. 17A, showing remote control by one of the other devices.

FIG. 18 illustrates example communication related fault mode conditions.

FIG. 19 illustrates example data integrity related fault mode conditions.

FIG. 20 illustrates example ventilator settings related fault mode conditions.

FIG. 21 illustrates example alarm related fault mode conditions.

FIG. 22 illustrates an example method relating to fault mode condition analysis implemented by a VCD including control by the VCD.

FIG. 23 illustrates an example method relating to fault mode condition analysis implemented by a VCD, including details relating to communication related fault mode condition analysis.

FIG. 24 illustrates an example method relating to fault mode condition analysis implemented by a VCD, including details relating to data integrity related fault mode condition analysis.

FIG. 25 illustrates an example method relating to fault mode condition analysis implemented by a VCD, including details relating to ventilation settings related fault mode condition analysis.

FIG. 26 illustrates an example respiratory distress ventilator with non-ventilation and ventilation modes.

FIG. 27 illustrates an example of respiratory distress ventilator ventilation capabilities.

FIG. 28A illustrates an example front of a respiratory distress ventilator, with included controls including a power switch and a communication light.

FIG. 28B illustrates an example front of a respiratory distress ventilator, with included controls including a power switch, a remote control light, and a smaller display.

FIG. 28C illustrates an example front of a respiratory distress ventilator, with included controls including a power switch, a remote control light and a larger display.

FIG. 29 illustrates an example of ventilation control software including CLC capability.

FIG. 30 illustrates an example emergency care environment in which context sensitive guidance (CSG) is provided.

FIG. 31 illustrates an example of components of a system for providing of CSG.

FIG. 32A-C illustrate an example method for providing CSG.

FIG. 33 illustrates an example of a companion device configured to provide a device view.

FIG. 34 illustrates an example of a combined ventilation system/patient monitor-defibrillator device view user interface at the companion device.

FIG. 35 illustrates an example of a medical device data screen.

FIG. 36 illustrates aspects of an example of the mechanical ventilation apparatus for a ventilation system such as a respiratory distress ventilator.

FIG. 37 illustrates aspects of an example pneumatic system that can be used with a portable ventilator.

FIG. 38 illustrates aspects of example external gas supply systems that can be used with a portable ventilator.

FIG. 39 illustrates aspects of example patient circuits that can be used with a portable ventilator.

FIG. 40 illustrates an example of components of various devices described with reference to FIGS. 1-39.

### DETAILED DESCRIPTION

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

In some instances, variations of a term may be utilized that may refer to the same or similar concepts, and certain terms may have meanings that are informed by a particular context. Generally, sending, receiving, or transmitting of data may include by wired and/or wireless connection, and/or within one or more wired or wireless networks. Furthermore, sending from a first entity to a second entity, or to be received by the second entity, can include sending from the first entity to the second entity, or to be received by the second entity, directly from the first entity to the second entity, or indirectly via one or more intermediary entities.

Respiratory distress may include, for example, any form of respiratory or breathing difficulty, impairment or problem, including respiratory failure. Respiratory parameter data can include data relating to respiratory, pulmonary or lung related parameters, as may, for example, be associated with respiratory, pulmonary or lung related characteristics, attributes or functions. Respiratory parameter data can include, for example, data related to pulmonary associated pressure, flow, volume or capacity related parameters, including parameters that may be measured using one or more flow sensors, pneumotachometers or spirometers. Respiratory parameter data can include, for example, data relating to respiratory mechanics, such as respiratory compliance (Crs), respiratory elastance (E) and respiratory resistance (Rrs). Respiratory parameter data can also include, for example, parameters such as vital capacity (VC), forced vital capacity (FVC), forced expiratory volume (FEV) at timed intervals (e.g., between 0.5 and 10 seconds, less than 0.5 second, 0.5 second, 1.0 second or FEV1, 2.0, 3.0 seconds, 4.0 seconds or 5.0 seconds), forced expiratory flow (FEF) at, e.g., 10%-90% capacity, such as 25%-75% or FEF25-75, peak expiratory flow rate (PEF or PEFR) and maximum breathing capacity (sometimes called maximal voluntary ventilation or MVV). Respiratory parameter data may be presented in various different ways or forms, such as may include, for example, in raw terms, such as liters or liters per second, or as percentages. Respiratory parameter data may also be presented, for example, as “predicted” values, such as percent predicted, which can, for example, include results as a percentage value in connection with a reference, average, or other “predicted” value. “Predicted” values may, in some cases, be associated with patients or hypothetical patients of one or more similar characteristics.

A respiratory status may, for example, relate to, identify, or indicate the presence or absence of, one or more respiratory, pulmonary or lung related conditions, and may include RD. A condition may include a state, disease, or problem, or several thereof, or a type, group or category thereof. A respiratory status may include an etiology relating to a respiratory condition or a non-respiratory condition (e.g., respiratory distress associated with acute heart failure). A non-respiratory condition may, for example, be associated with one or more non-respiratory systems, e.g., cardiac, endocrine, exocrine, circulatory, immune, lymphatic, nervous, muscular, renal, skeletal, or others. Additionally, a respiratory status may include one or more determined, assessed, estimated, probable or possible conditions, or determined, assessed, probable or possible associated etiologies. A respiratory status may also include data associated any of the foregoing, which may be called respiratory status data.

A ventilator may include, for example, any device or system capable of delivering ventilation, whether such delivered ventilation is controlled internally, remotely, or with aspects of both.

Various ventilation parameter related terms or abbreviations, including fraction of inspired oxygen (FiO2), positive end-expiratory pressure (PEEP) and others, may refer to ventilation related settings, even though the word “setting” may or may not be stated. Furthermore, reference to a ventilation parameter or parameter setting may be used to refer to the parameter in a conceptual or definitional sense, or the value associated with a particular setting (e.g., “PEEP of 5 cm H2O”). A user may include an individual operating, supervising or in whole or in part responsible for operation of a device such as a respiratory distress ventilator, even if, during a particular period of time while the device is operating, the user may not be interacting with the device.

An alert or alarm may be presented for the attention of a user, such as by being visually or audibly presented, such as via a display, graphical user interface (GUI) or speaker of a device, and may or may not be included in context sensitive guidance (CSG). However, an alert or alarm may also include an alert or alarm condition that is algorithmically identified, recognized or determined by a computerized device, and not necessarily presented or displayed. The term optimizing may include, for example, improvement or improved operation in one or more aspects, for example, relative to an actual, potential or hypothetical less optimized situation or less optimized operation. The term adjusting can include changing as well as not changing or maintaining without change, as may be appropriate. A determined parameter value can include a determined estimated or determined approximated value for the parameter. The term monitoring can refer to or include, for example, monitoring or tracking performed by a computerized device utilizing one or more algorithms and not by a person or user, or monitoring by a person or user, or both. The term continuous can include, among other things, on a periodic basis (with identical or different periods), on a frequent basis, on a repeated basis, or cyclically, for example.

Some embodiments of the present disclosure provide, include, or include use of, apparatuses, systems and methods for use in care of patients experiencing, or having experienced, respiratory distress, such as may include a respiratory distress ventilator. In various embodiments, or modes of operation, a respiratory distress ventilator may include, for example, a device that may control itself or may be remote controlled, such as by a remote control device or system, or may include aspects of both.

In some embodiments, a controller of a respiratory distress ventilator internally signals a mechanical ventilation system (or apparatus) of the respiratory distress ventilator in order to implement control thereof, whether such internal signaling implements internal control or remote control. Internal control includes a controller of a respiratory distress ventilator internally signaling the mechanical ventilation system of the respiratory distress ventilator to implement control according to a setting value (or more than one setting value) that the controller determines, even if the determined setting value is determined based at least in part on a setting value of a received request/command. Remote control includes the controller of the respiratory distress ventilator internally signaling the mechanical ventilation system of the respiratory distress ventilator according to a setting value received by the controller via a request/command from a remote control device or system.

In some embodiments, a respiratory distress ventilator may be capable of either internal or remote control, or operation with aspects of both, and may be capable of determining which, or what particular state or mode, is employed or used at a predetermined or particular time. Furthermore, a respiratory distress ventilator may be capable of switching between modes or states of operation, such as may include an internal control mode, a remote control mode, or a mode that includes aspects of both.

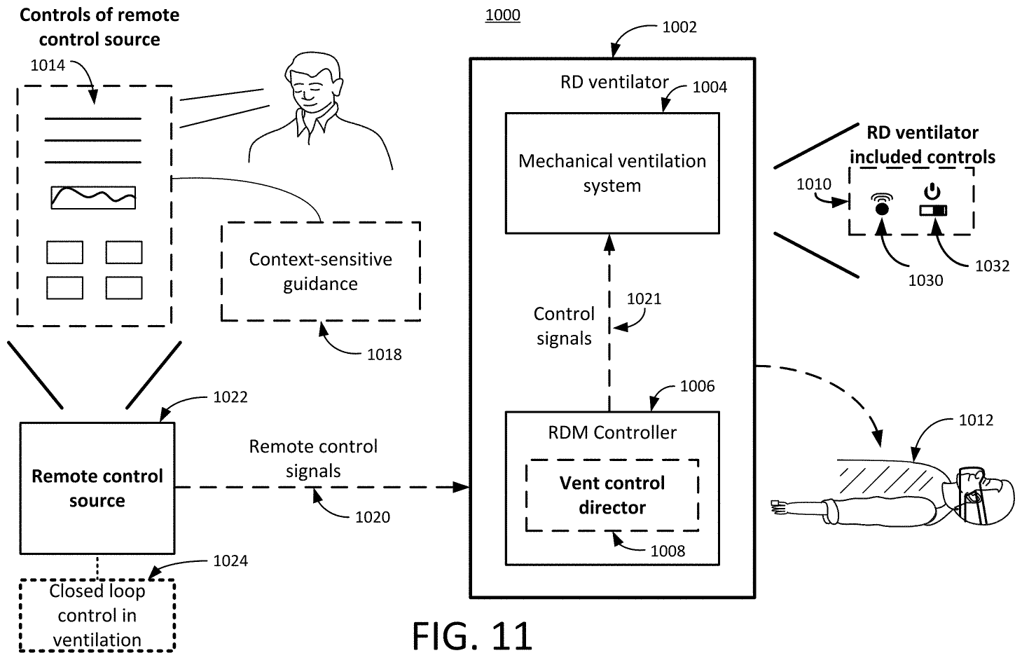

A controller can include physical aspects such as one or more processors and one or more memories, as well as software based aspects, which may include one or more programs, algorithms or software based aspects. The software based aspects may, for example, be stored, in the one or more memories of the controller, accessed by the one or more processors of the controller and used or executed by the processor. In various embodiments, a ventilation control director (VCD) may be or include, for example, software based aspects of one or more controllers, such as a respiratory distress ventilator controller, or may be or include both software and hardware aspects, such as may include processor and memory aspects. In some embodiments, a VCD may be used in management and implementation of aspects of control of mechanical ventilation provided by a respiratory distress ventilator. This may include management and implementation of internal control, remote control or aspects of both. In some embodiments, a VCD may be used in assessing or determining whether internal control or remote control is implemented, or aspects of both, as well as in implementing control accordingly, which may include switching between internal or remote control, or aspects of both, for example. In some embodiments, a VCD may determine a control mode, such as internal control or remote control, based on whether a fault mode condition is detected at a predetermined time. Further description relating to embodiments of a VCD are provided reference to FIGS. 11-25.

The term controls may include physical controls, display/GUI based controls, or both. Controls may, for example, allow user interaction with a device or system, such as may affect operation of the device or system, which may include, for example, making selections or providing input to set, select, change, increase, decrease, confirm or override implementation of a parameter setting value or a mode. A device or system with controls may, in various aspects, operate without user interaction, with user interaction, or with aspects of both. This may include operation that may proceed, in various aspects or instances, without user interaction, but may permit of, or require, user interaction in some aspects or instances. A display/GUI may include controls, and/or controls may include a display/GUI.

In some embodiments, a set of controls, such as of a particular device, may include one or more respiratory aspects. Respiratory aspects of controls may include, for example, display/GUI aspects relating to any aspects of ventilation, ventilation parameters or aspects relating to respiration, respiratory distress or respiratory parameters. Respiratory aspects of controls may or may not include CSG.

Herein, remote control may include control, or aspects of control, provided from outside of, or separately from, a controlled device or system, such as by wired or wireless signaling or communication, such as may include, for example, Bluetooth or ultrawide band. As such, remote control may be implemented via a wired only connection that connects a remote control device and a remotely controlled device, or by a wireless only connection, or by a combination of wired connection and wireless connection, for example. Remote control, such as remote control by a remote control device or system, may include control with or without aspects of user interaction with the remote control device or system, such as, for example, by the user using controls of the remote control device or system. Internal control, such as a device internally controlling itself, may or may not include aspects of user interaction with the internally controlled device, such as, for example, by the user using controls of the internally controlled device. User interaction may include, for example, a user using controls to input, set, select, change, increase, decrease, confirm or override implementation of a parameter setting value or a mode. User interaction may or may not be prompted, guided, or interactively guided, e.g., via CSG, one or more GUI based prompt messages, alarms or alerts that may relate, for example, to patient parameters or ventilation parameters, such as may relate to ventilator operation, performance or components, such as sensors.

Any of various types of gas movers/gas flow generators may be used in various embodiments of a respiratory distress ventilator. These may include, for example, blowers/turbines, compressors, pneumatic regulators, bellows or pistons, for example.

In some embodiments, a respiratory distress ventilator is configured for integration with one or more other devices or systems, such as onsite or offsite remote devices, systems or interfaces, aspects of which may or may not be included in various embodiments. For example, in some embodiments, the respiratory distress ventilator may couple, in a wired and/or wireless fashion, with a device such as a patient monitor or critical care monitor (CCM). In some embodiments, for example, a defibrillator, respiratory distress ventilator, or other device, such as another medical device, monitoring device or computing device, may be, include or function as a CCM. Furthermore, in some embodiments, the respiratory distress ventilator may couple with one or more computing systems, computers, computing devices or portable computers, which can include, for example, a cloud-basing computing system, computer, notebook computer, tablet, touch-based device, smartphone, wearable device or implantable device, among other things.

In some embodiments, smaller size, lighter weight and/or greater simplicity may be desirable or optimized in portable devices including a respiratory distress ventilator. Optimization may take into account various factors such as the actual, predicted, likely or anticipated setting, context, environment or application, such as pre-hospital contexts. Optimization may also take into account allocation of roles of various devices in such contexts. Some embodiments provide a respiratory distress ventilator that is optimized in various ways, as well as related apparatuses, systems and methods. The respiratory distress ventilator may be smaller and/or lighter than various other ventilation devices or systems used, for example, in pre-hospital care. Furthermore, the respiratory distress ventilator may be optimized for users with limited training or experience. Still further, the respiratory distress ventilator may be optimized for use in environments that may include other integrated devices or systems. Additionally, some embodiments provide a system of devices, including a respiratory distress ventilator, in which each device and the system as a whole are optimized and capable of optimized operational integration, in view of such factors and potentially others.

For example, the space immediately surrounding a critical care patient can be very crowded with equipment or items such as, for example, hoses, tubes, wires and various devices. It can be beneficial or advantageous to minimize, for example, the spatial impact of a ventilator. In some embodiments, for example, a respiratory distress ventilator may provide advantages by allowing modularization of, or allowing a higher degree of modularization of, a system including a ventilator, which may allow for a much smaller, simpler ventilator close to the patient. Furthermore, in some embodiments, the controls in or of a remote control device or system may be some variable distance from the patient (e.g. between 0-3 feet, between 3-6 feet, between 6-9 feet, between 9-12 feet, further than 12 feet, or offsite). Some embodiments provide benefits or advantages to patient care by, for example, simplifying the immediate clinical environment of the patient. For example, in some embodiments, even if the combined weight and/or size of a combination of a ventilator and different remote control device, together, are the same or similar to a system in which the controls are provided in or with the ventilator, the combination of the ventilator and the different remote control device may be more usable, or more practically or optimally usable, in various clinical contexts, for example, since only a relatively small ventilator may be required to be at the patient's side or very close to the patient. Some embodiments provide respiratory distress ventilators that provide these advantages.

In some embodiments, an overall environment may include a respiratory distress ventilator that may couple and operationally integrate with one or more other devices, each of which may provide certain roles or functionality in the overall environment. These roles may relate to, for example, device and patient related sensing, operation, processing, software, algorithms, applications, data storage, communication, integration (of devices and systems, roles, functions, and physical, software based and conceptual components and aspects), user interaction and guidance, and patient related assessments or interventions. Within the integrated environment, roles of each device may be optimized, taking into account, for example, factors relating to each device or system as well as the overall environment and anticipated settings. Such optimization may also take into account factors relating to the need for the respiratory distress ventilator to be of a particular or sufficiently small size, light weight, and/or degree of simplicity or ease of portability or use. In some embodiments, a respiratory distress ventilator augments the ability to accurately assess or screen a patient's condition, to determine or select, and initiate and deliver, an effective intervention, and/or to ongoingly control or adjust, or assist in control and adjustment of, parameters relating to an applied intervention. Furthermore, this augmentation may be optimized in view of the overall environment, available devices, systems or users at a given time or time period, and available communication between them at a given time or time period, as well as changing aspects thereof over time.

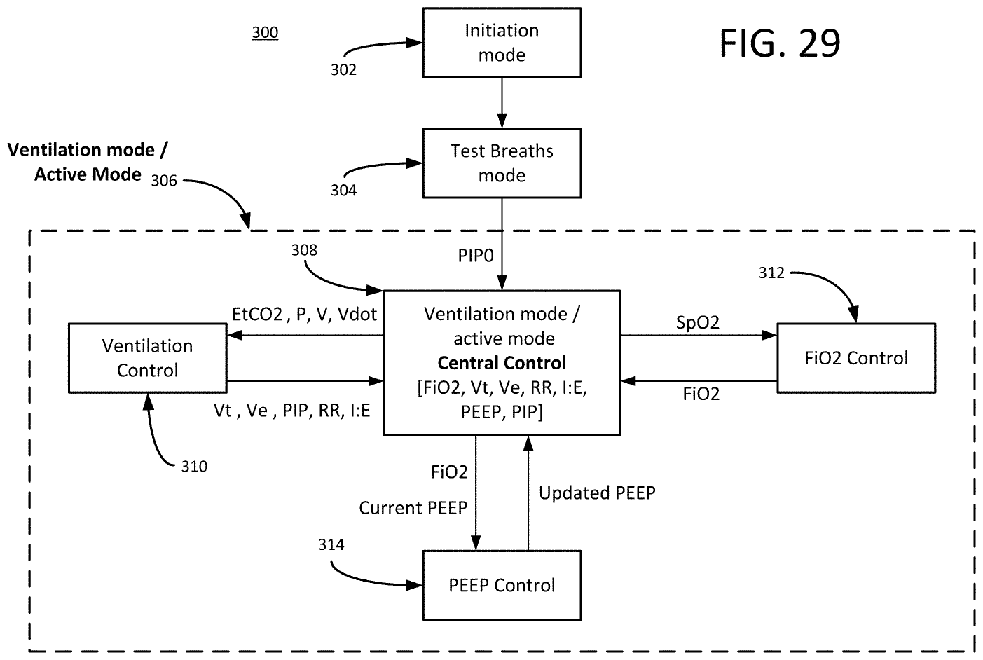

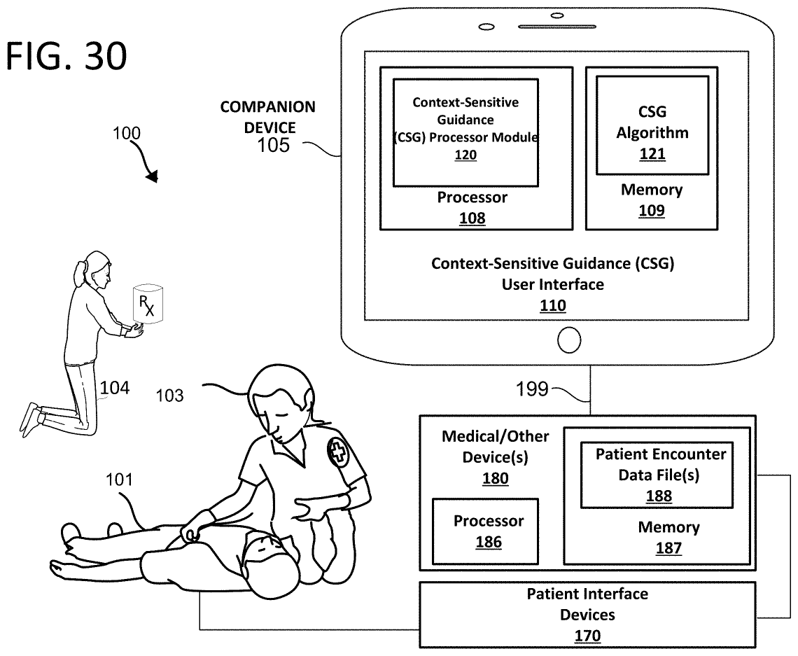

In some embodiments, for example, one or more devices or systems coupled with a respiratory distress ventilator, or integrated into the environment thereof, may supply, or partially supply, components or other aspects that may augment or enhance the operation of the respiratory distress ventilator, and/or otherwise might be, or need to be, included with or in the respiratory distress ventilator. Some such aspects may include physiological and operational signaling, sensing or measuring aspects. Other aspects may relate to power, communication, device recognition, authentication or coupling. Other aspects may relate to processing, hardware and software, such as for data processing and management, data and device integration, or operational aspects such as closed loop control (CLC), examples of which are described with regard to FIG. 29. Other aspects may related to patient respiratory or status assessment, diagnostic screening, diagnosis, treatment or intervention. Other aspects may include controls and control relating to the respiratory distress ventilator (physical, display/GUI based, or both), as well as features thereof. Still other aspects may include determination and/or presentation of context sensitive guidance (CSG), as described further below with reference to, e.g., FIGS. 30-35.

In some embodiments, a respiratory distress ventilator may have a set of (one or more) included controls, while one or more remote control devices, systems or monitors may also have a set of (one or more) remote controls relating to the respiratory distress ventilator. A set of remote controls may for allow remote control of the respiratory distress ventilator, such as may relate, for example, to aspects of operation of a mechanical ventilation system of the respiratory distress ventilator. In various embodiments, a set of included controls of a respiratory distress ventilator may vary across a spectrum of possibilities.

In various embodiments, a set of remote controls may include respiratory aspects that are separate from other aspects, or may include respiratory aspects that are mixed, combined or integrated, for example, into a larger display/GUI or set of controls that may include non-respiratory aspects as well. Furthermore, in various embodiments, a set of remote controls may be separate or separable from other aspects of controls, or may be integrated with other aspects. In some embodiments, the respiratory distress ventilator may have no included controls at all, or may have a limited set of included controls. For example, any or many controls relating to the respiratory distress ventilator may be provided by one or more sets of remote controls of one or more other devices, systems or monitors. In some embodiments, a set of remote controls, or a portion thereof, may, in whole or in part, correspond to, emulate, mirror, duplicate, replicate, simulate, or be in any of varying degrees similar to a set of included controls of the respiratory distress ventilator. Furthermore, in some cases in which the respiratory distress ventilator has no controls or a limited set of controls, a set of remote controls may, to some degree, be similar to or replicate a hypothetical set of included controls that might be included in a respiratory distress ventilator.

As such, in various embodiments, a set of remote controls may be the same, may replicate, may be similar to some degree, or may be different than a set of included controls. Furthermore, various particular individual controls or aspects of the two sets may include ones that are the same or different, or have the same or different scopes, and which may overlap, partially overlap or not overlap. In various embodiments, the set of included controls may be, in at least some aspects, more limited or more simplified than the set of remote controls, or vice versa. For example, a set of included controls may or may not be, or include, in whole or in part, a subset of the set of remote controls, or vice versa. The set of remote controls may include individual controls or aspects that are, or are not, similar or identical to, reflect or mirror individual controls or aspects of the included controls. The set of remote controls may provide visual or GUI features or information relating to, or allowing control of, some or all of the aspects, modes, settings or other parameters that are reflected by or incorporated into the included controls.

In some embodiments, curating, selecting or limiting the set of included controls of a respiratory distress ventilator, such as to an optimized degree, may provide various advantages. Such advantages may include, for example, ease or range of portability (which may include being lightweight, being of small size or having a small footprint), practicality, ease or range of usability, simplicity of control, simplicity of user interaction, or simplicity of operation.

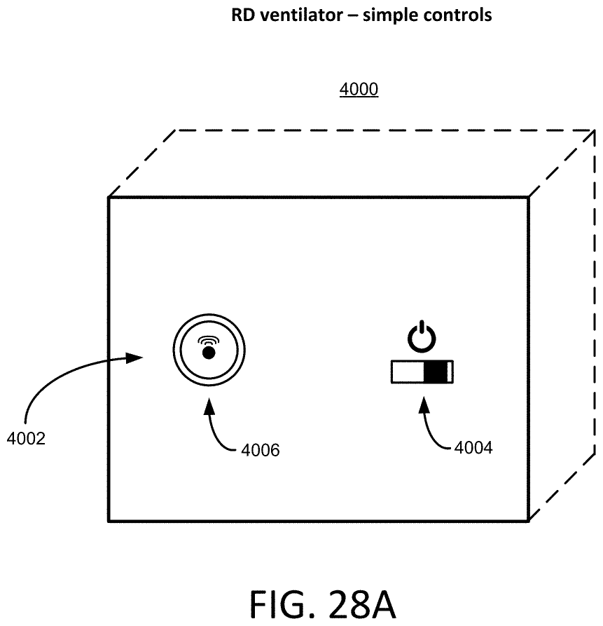

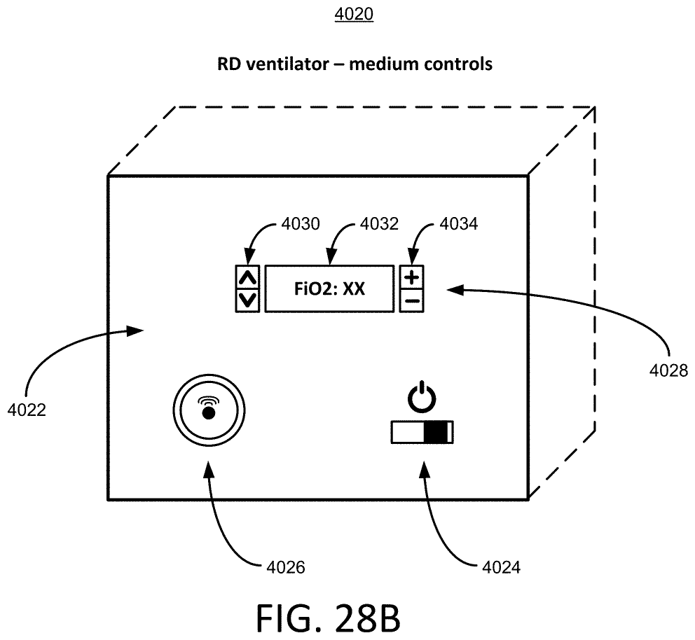

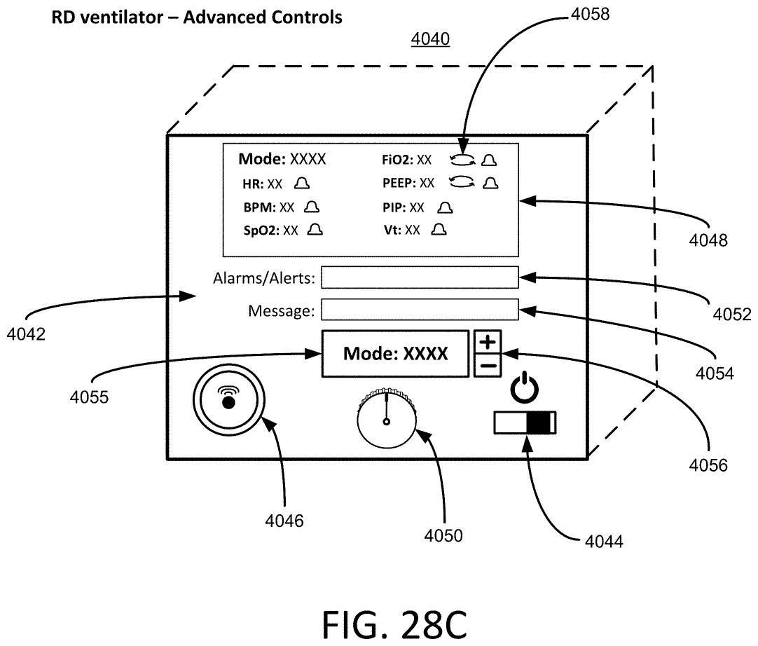

In some embodiments, the set of included controls of the respiratory distress ventilator may be optimized, for example, taking into account factors such as actual, anticipated or likely environments or settings, which can include considerations relating to locations, situations and use case scenarios. Other factors may relate to users and their training, experience and potential or likely degree of availability or distraction at a scene. Other factors may relate to patients or types of patients. Still other factors may relate to devices, systems, communication, reliability or speed of communication, or data and data management aspects. Some examples of controls, including display/GUI aspects, of a respiratory distress ventilator are described with reference to FIGS. 28A-C.

The set of remote controls of a remote control device may also be optimized, including taking into account the foregoing factors, as they apply to the remote control device, as well as other factors. For example, the set of remote controls may include a display/GUI, that is, to a selected or optimized degree, more inclusive, larger, broader in scope, more comprehensive, more sophisticated, more complex and/or more granular than the set of included controls of the respiratory distress ventilator. In some embodiments in which the set of remote controls is, for example, more inclusive or comprehensive than the set of included controls of the respiratory distress ventilator, control by or using the set of remote controls, or by the associated remote control device, may allow various advantages. These advantages may include, for example, greater, additional, more sophisticated, more complex, or more granular control, aspects of control, user based control, user interactivity, or user based control with greater available display/GUI. The greater available display/GUI may include, for example, information such as parameter values, alarms, alerts, user messages or CSG and user interactivity features.

In some embodiments, a respiratory distress ventilator may be capable of being remotely controlled, or may be capable of both remote control or internal control, or aspects of both, such as may include use of a controller of the respiratory distress ventilator. In some embodiments, for example, a respiratory distress ventilator may be capable of determining whether to enable and use remote or internal control, or aspects of both, and may be capable of switching between remote and internal control modes, or modes that include aspects of both. For example, in some embodiments, remote control, or aspects thereof, may be generally preferred when available or optimal, and internal control may be used only when external control, or aspects thereof, is determined to be unavailable, unsafe, not practical, or not optimal relative to internal control. In various embodiments, remote control may be determined to be unavailable or not optimal if, for example, there is no or insufficient communication with the remote control device or system, or other problems exist such as may relate to effective, optimal or safe control or operation of the respiratory distress ventilator by the remote control or by particular remote control requests/commands.

In some embodiments, remote control may include more complex or sophisticated forms or aspects of ventilation or ventilation control, such as may, for example, include CLC aspects, while internal control may include less complex or sophisticated control, such as continued use of current parameter values without adjustment, or use of default or backup parameter values. Furthermore, in some embodiments, a respiratory distress ventilator may include a controller that monitors and allows remote control within certain limits, parameters, or setting value ranges, but may override external control and utilize internal control if and when needed to maintain required setting ranges, for example.

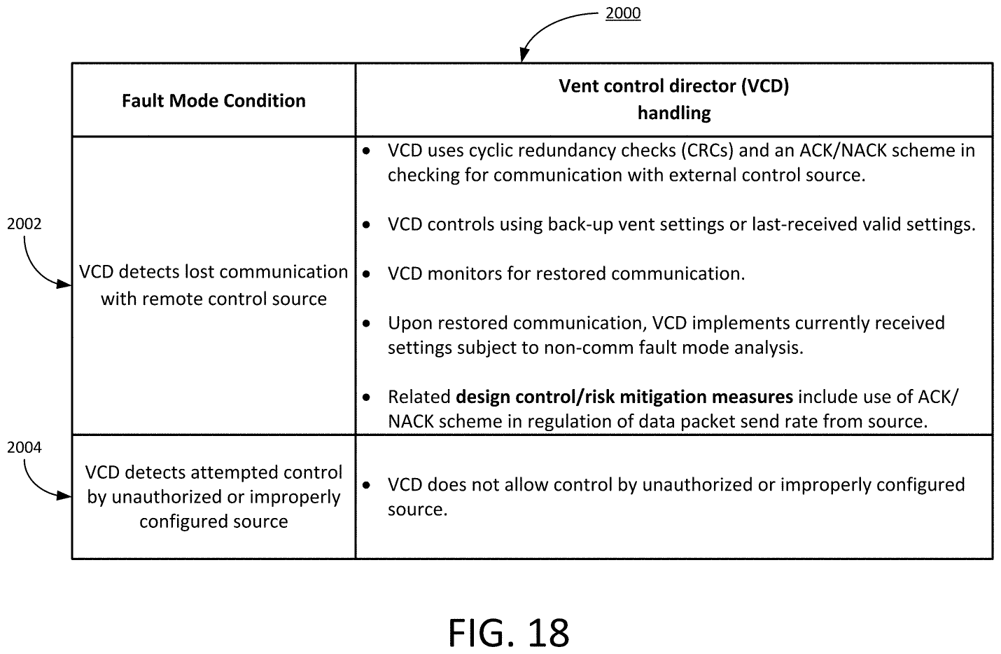

In some embodiments, a respiratory distress ventilator, or the controller thereof, may be configured to allow remote control at times, when a fault mode condition is determined not to exist. In some embodiments, a fault mode condition may include a condition that warrants utilizing internal control, or aspects thereof, as opposed to enabling or allowing remote control. In some embodiments, lack of, or insufficient, communication capability between the respiratory distress ventilator and remote control device or system may constitute a fault mode condition. For example, if such communication is determined not to exist (or be lost), or be insufficient or insufficiently reliable, the respiratory distress ventilator may use (or switch to) internal control (or an internal control mode). The internal control mode may effectively operate as a back-up mode or default mode in the event that control by the remote control device is or becomes unavailable or does not meet particular required conditions. In some embodiments, fault mode conditions may include conditions relating to the remote control device or data received from it, such as may relate, for example, to communication, data integrity, ventilator settings or alarm conditions, as described further with reference to FIGS. 18-21 herein.

In some embodiments, ventilation delivered by a respiratory distress ventilator may include use of various forms of CLC in ventilation. In some embodiments, such CLC may be available when remote control is available, when particular physiological sensing capability is available, or both. While the respiratory distress ventilator may deliver the ventilation, control of aspects of the delivered ventilation may be from a remote source, and the remote source may employ control aspects or features that may include or incorporate forms or aspects of CLC. Forms of CLC that may be available may include CLC relating to fraction of inspired oxygen (FiO2), which may be driven at least in part by measured patient oxygen saturation or SpO2. Forms of CLC may also include CLC relating to positive end-expiratory pressure (PEEP), which may, for example, be driven by current FiO2 as well as a current PEEP. This may include associated PEEP change eligibility rules and PEEP selection rules, further description of which is provided with reference to FIG. 29. For example, in some embodiments, a respiratory distress ventilator, and/or one or more remote control sources, may each include a controller with CLC capability (e.g. of FiO2, PEEP or both, and/or other parameters). As such, in various embodiments, a controller of a respiratory distress ventilator may be capable of internal control including CLC, or of implementing remote control that includes CLC, or both. In some embodiments, a respiratory distress ventilator that is capable of internal control or of being remote controlled may only provide mechanical ventilation including CLC capability, or some forms of CLC capability, when being remote controlled.

In some embodiments, a respiratory distress ventilator may include one or more sensors, flow sensors, pneumotachometers or spirometers, such as may be coupled with inspiratory or expiratory patient circuits or patient circuit limbs of the respiratory distress ventilator that may be capable of sensing signals that can be used in determining at least one patient respiratory parameter. This, in turn, may be used in patient respiratory status determination, screening or diagnosis, or in determining an appropriate intervention. For example, included flow sensors or pneumotachometers may be used both in connection with sensing and measurement associated with a patient's respiratory function as well as in connection with respiratory distress ventilator performance and operation.

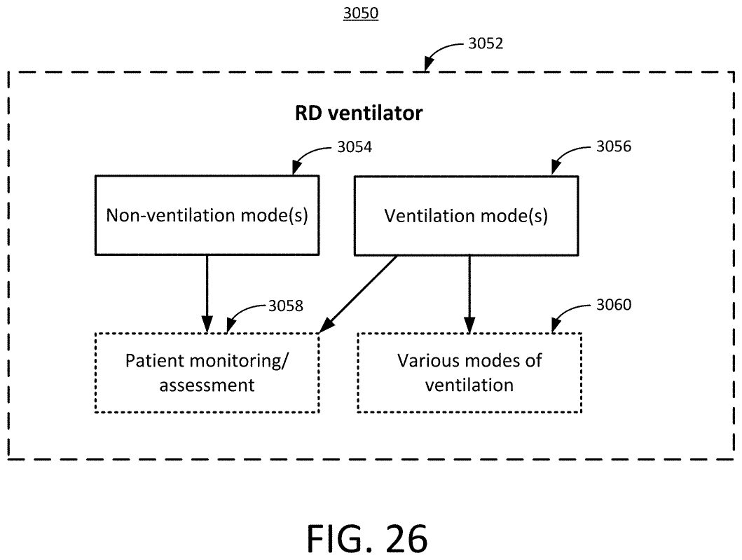

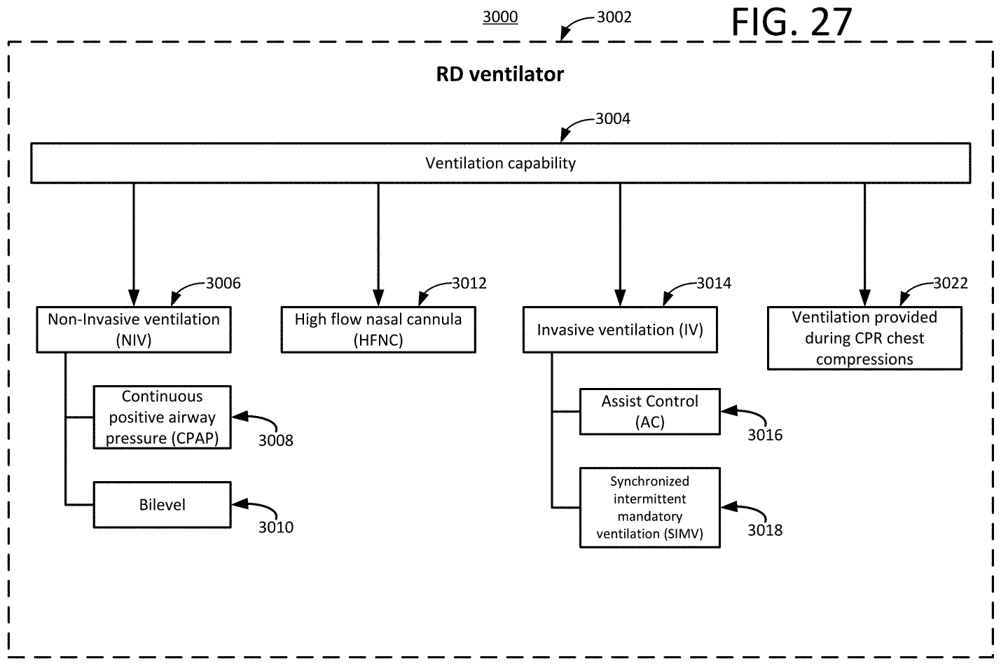

In some embodiments, a respiratory distress ventilator may include one or more non-ventilation modes in which ventilation is not provided, as well as one or more ventilation modes in which ventilation, of various possible types, is provided. Further details with regard to such modes is provided with reference to, e.g., FIGS. 26-27.

In some embodiments, user interactive CSG is determined and provided to a user. Details with regard to CSG are described further with reference to, e.g., FIGS. 30-35.

In some implementations, for example, after a user with a respiratory distress ventilator arrives at a scene with a patient, the respiratory distress ventilator is initially used in obtaining patient respiratory parameter data, which is used in determining a respiratory status of the patient (at the respiratory distress ventilator or elsewhere), and may be used in determining CSG (at the respiratory distress ventilator or elsewhere). The user may be alerted (at the respiratory distress ventilator or elsewhere), such as via CSG, when the patient's respiratory status is being assessed. Furthermore, if additional physiological monitoring is needed, it may be initiated, or the user may be prompted, such as via CSG, to initiate it. If and when it is determined that further monitoring is not appropriate, such as may be determined based at least in part based on the patient's physiological parameters being within normal ranges, the user may be prompted (at the respiratory distress ventilator or elsewhere), such as via CSG, to discontinue use of the respiratory distress ventilator in monitoring the patient. If and when ventilation is appropriate, the user may be so prompted (at the respiratory distress ventilator and/or elsewhere), such as by CSG, and, potentially after the user confirms, ventilation in a particular determined or selected mode may be initiated by the respiratory distress ventilator. Furthermore, during ventilation provided by the respiratory distress ventilator, patient respiratory parameters and respiratory status may continue to be monitored. If ventilation is no longer called for, such as may be determined based at least in part on the patient's physiological parameters, then respiratory distress ventilator ventilation may be discontinued, or the user may be prompted to discontinue it. Alternatively, if another mode of ventilation is determined to be more appropriate or optimal, the user may be prompted to switch to it.

Furthermore, in some embodiments, the respiratory distress ventilator may be used in detection of the need for intervention, whether a mode of ventilation or otherwise. If intervention is determined to be needed, CSG may be used in guiding the user in managing the patient as needed.

The following is a simplified example event including use of a respiratory distress ventilator in an emergency care situation. Based at least in part on patient physiological parameters including cardiac disease history and monitored parameters including SpO2, respiratory rate (RR) and Crs, a patient may be determined to have a respiratory status that includes respiratory distress related to acute heart failure. It may be determined to be appropriate to initiate continuous positive airway pressure (CPAP) ventilation with continuous monitoring of parameters including SpO2, end-tidal carbon dioxide (EtCO2) and minute volume (Ve). The user may be guided accordingly via determined CSG. If the patient's condition deteriorates further in spite of this treatment, as may be determined based at least in part on the continuously monitored SpO2, EtCO2 and Ve, among other things, it may be determined to be appropriate to initiate bilevel ventilation, and the user may be guided accordingly via CSG. Throughout the foregoing example event, displays/GUIs may be provided on devices in the environment, such as a tablet, defibrillator, the respiratory distress ventilator, and/or one or more other devices or systems, that may include integrated respiratory aspects such as may include respiratory parameter values, alerts, alarms, messages or CSG. Furthermore, the respiratory distress ventilator may be internally controlled, remote controlled, controlled with aspects of both, or internally controlled during some time periods and remotely controlled during other time periods, for example.

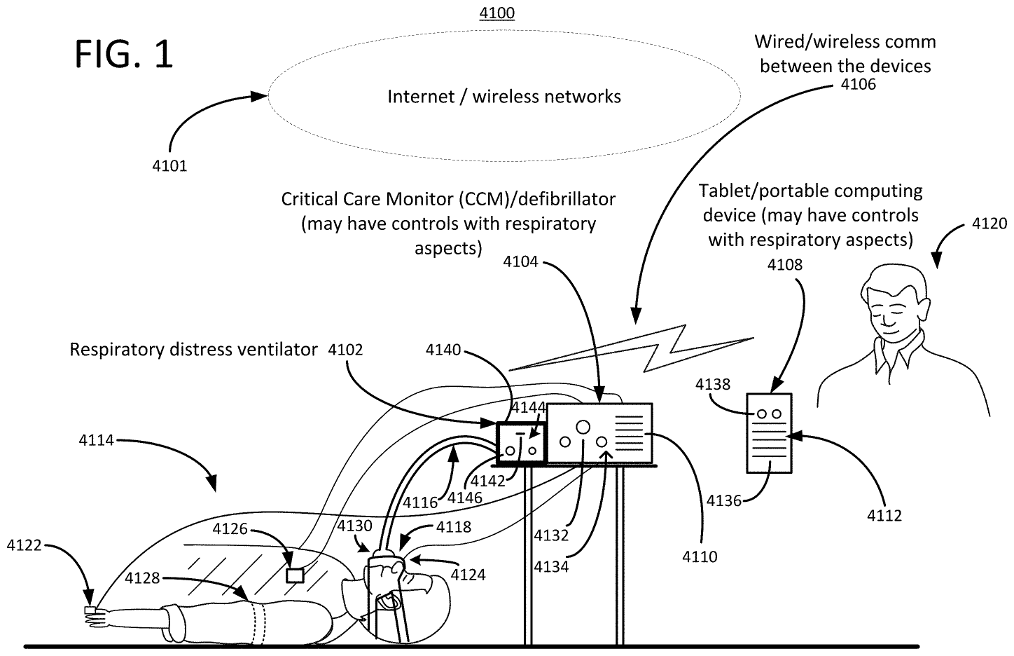

FIGS. 1-7 illustrate example environments including a respiratory distress ventilator, among other devices and systems, according to various embodiments. The various devices and systems may be communicatively coupled by wired and/or wireless connection. Many different combinations of devices and systems, and roles for each, may be used in various embodiments, only a few examples of which are illustrated in FIGS. 1-7.

In FIGS. 1-7, in various implementations, a depicted respiratory distress ventilator may be capable of being remote controlled by one or more of other depicted devices or systems, which may include, for example, a CCM, defibrillator, medical or monitoring device, computing device, portable computing device, tablet, smartphone, or offsite monitor system. In some embodiments, the respiratory distress ventilator may be internally controllable or remotely controllable, and may be able switch between internal and remote control modes.

In various embodiments, particular sensing capabilities and components may be distributed in various ways between the various devices or systems in an environment. These may include, for example, one or more electrodes, capnographic sensors, pulse oximeters, flow sensors, pneumotachometers, spirometers, pressure sensors, barometric sensors, humidity sensors, oxygen sensors, temperature sensors, electrical or magnetic sensors, light/electromagnetic spectrum/optical sensors, blood pressure monitors, heart rate monitors, electrocardiogram (ECG) sensors and others. Sensing capabilities and components may relate, for example, to patient parameters and various patient systems, such as respiratory and circulatory systems. Sensing capabilities and components may also relate to parameters associated with devices and their operation, including a respiratory distress ventilator and ventilation, or a defibrillator and electrotherapy, among other things. In some embodiments, any of various sensing capabilities, such as those of a respiratory distress ventilator, CCM/defibrillator or tablet, may instead be provided by one or more other devices, or may be distributed between multiple devices. Also, in various embodiments, various sensing components, or all or part of a patient circuit, may or may not be considered to be part of a respiratory distress ventilator, or other device, even if they are connected thereto. Furthermore, in various embodiments, roles and functions of the various devices may be distributed differently between the devices.

FIG. 1 illustrates an example emergency care environment 4100 including a respiratory distress ventilator 4102, a CCM 4104, such as a CCM/defibrillator, and a tablet 4108 or other portable computing device such as, e.g., a smartphone or notebook computer. A user 4120 is also shown, which user 4120, and/or one or more other users, may monitor or interact, for example, with the patient 4114, the respiratory distress ventilator 4102, the CCM/defibrillator 4104 and or the tablet 4108. In the embodiment depicted, the devices 4102, 4104, 4108 are coupled with each other by wired and/or wireless connection 4106, such as including over the Internet and/or one or more wireless networks 4101. However, in some implementations, at various times, such connection might be unavailable, lost, limited, or of differing speeds. Furthermore, their operation may be integrated in various ways.

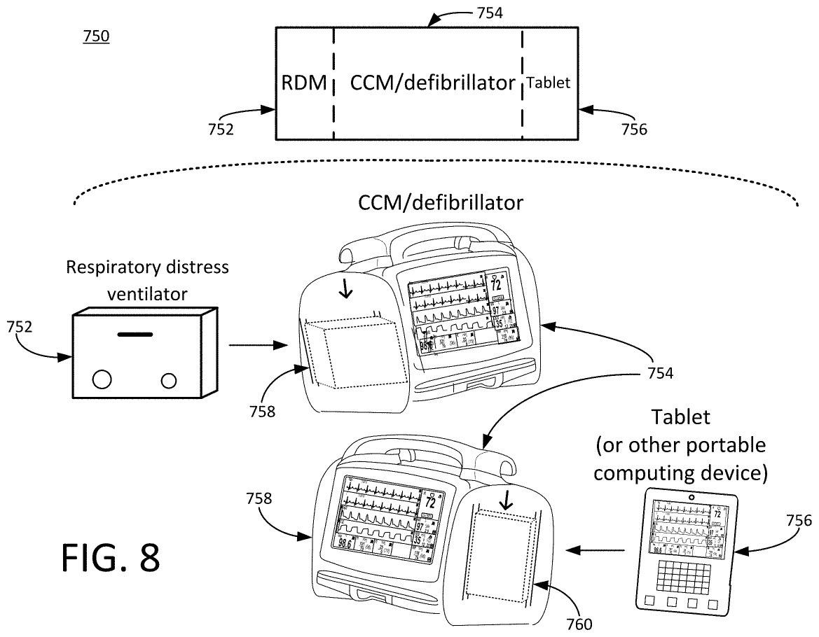

In various embodiments, devices, including the respiratory distress ventilator 4102, may have housings that may be separate and spaced from each other, that are adjacent or touching, or that are physically or mechanically coupled, connected or attached (fixedly or movably). Some examples of the foregoing are described with reference to FIG. 8.

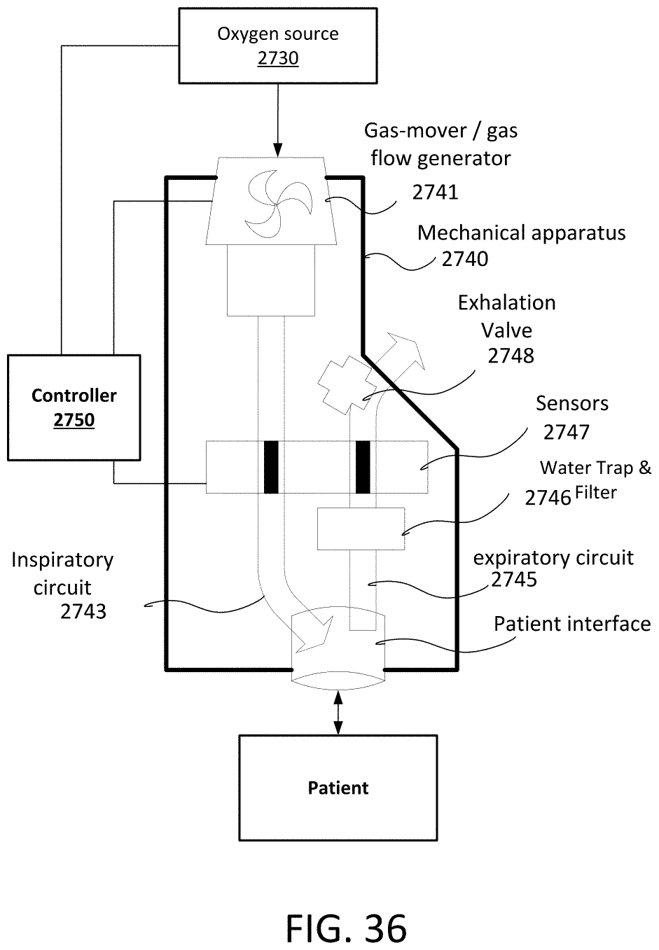

When delivering mechanical ventilation, the respiratory distress ventilator 4102 may provide breathing gas to a patient 4114 via a mechanical ventilation apparatus (examples of which are described with reference to, e.g., FIGS. 36-38) including a gas flow generator (such as a blower, turbine, compressor, pneumatic regulator, bellows, piston or device based thereon) and a gas delivery apparatus including a patient circuit 4116 that includes a facemask 4118. However, in some embodiments, ventilation may be provided via intubation or other patient interface such as nasal cannula rather than via a facemask 4118.

In the example depicted in FIG. 1, the CCM/defibrillator 4104 includes a pulse oximeter 4122 for measuring patient SpO2, a capnographic sensor 4124, which may be used in measuring EtCO2, a blood pressure sensor/monitor 4128, electrodes 4126 that may be used in providing electrotherapy to the patient 4114, and may include various other components such as one or more flow sensors, pressure sensors, airway sensors or spirometers. The respiratory distress ventilator 4102, CCM/defibrillator 4104, tablet 4108 and other devices may also include various sensing, measuring, computerized, electrical, mechanical, coupling and output aspects or components, including power supplies and batteries.

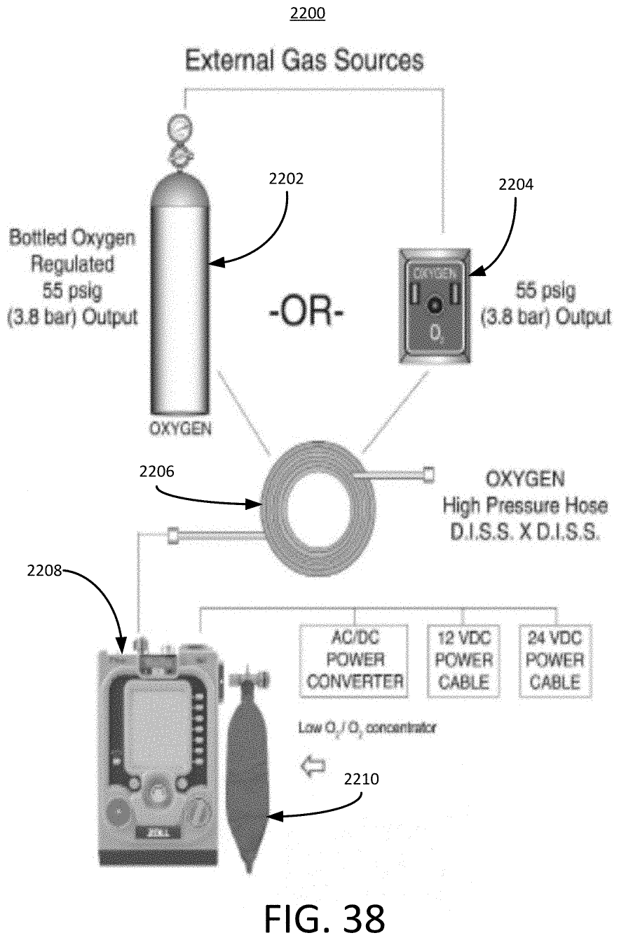

In the example depicted in FIG. 1, the respiratory distress ventilator 4102 may be coupled with a supplemental oxygen (O2) source, some examples of which are described with reference to FIG. 38. In various embodiments, the respiratory distress ventilator 4102 may be capable of supplying oxygen, using the supplemental oxygen source, in a number of different ways, or the respiratory distress ventilator 4102 may be capable of supplying oxygen in any one of several different ways. In various embodiments, the manner in which oxygen is supplied may be determined without user selection, or may be selected by a user, and may or may not require user confirmation.

In some embodiments, a reservoir bag may be used that allows entrainment of oxygen from a low pressure oxygen source. For example, a user may adjust the flow rate of oxygen based at least in part on current SpO2, which may be monitored by one or more of the devices 4102, 4104, 4108. In the embodiment depicted in FIG. 1, the CCM/defibrillator 4104 continuously monitors SpO2. In various embodiments, current SpO2 may be displayed for viewing by the user via a display/GUI of one or more of the devices 4102, 4104, 4108.

In some embodiments, the respiratory distress ventilator 4102 includes and uses a variable rate regulatory valve 4140, in which an oxygen output rate allowed or facilitated by the variable rate regulatory valve 4140 may be varied and changed to a particular oxygen flow rate of a range of possible oxygen flow rates, for example, from 0 l/min to 200 L/min (or, e.g., up to 220, 250 or 275 L/min). The variable rate regulatory valve 4140 may be used in providing a variable and controllable oxygen flow rate for gas provided by the respiratory distress ventilator 4102 during the providing of mechanical ventilation. In the embodiment depicted in FIG. 1, the variable output regulatory valve 4140 may attach to a gas inlet of the respiratory distress ventilator 4102, a high pressure oxygen source, and the CCM/defibrillator 4104 (which monitor SpO2), for example.

In some embodiments, the variable rate regulatory valve 4140 may be controlled automatically or without need for user interaction. For example, this may be based at least in part on the patient's continuously monitored SpO2 and in accordance with an FiO2 setting or adjustment that may be determined based at least in part on the current SpO2. In some embodiments, this arrangement may be used in providing CLC of FiO2 (FiO2 CLC). In some embodiments, a portable oxygen concentrator (POC) may be used. FiO2 CLC may be used in controlling and regulating the output of the POC for entrainment of oxygen into the respiratory distress ventilator 4102 to be used in gas delivered by the respiratory distress ventilator 4102 during mechanical ventilation. Furthermore, in some embodiments, including embodiments in which the variable output regulatory valve 4140 or a POC is used, CLC of PEEP (PEEP CLC) may also be included in control of the respiratory distress ventilator 4104. In some embodiments, PEEP CLC may be based least in part on a current FiO2 setting and a current PEEP setting. Examples of embodiments of FiO2 CLC and PEEP CLC are provided with reference to FIG. 29. In various embodiments, control with FiO2 CLC and/or PEEP CLC may be provided internally by the respiratory distress ventilator 4102 or by a remote control device for the respiratory distress ventilator 4102, such as the tablet 4108 or the CCM/defibrillator 4104.

In some embodiments, the respiratory distress ventilator 4102 may operate, or may have one or more modes of operation, that do not include, or at times or during certain periods of time do not include, delivering mechanical ventilation. Furthermore, the respiratory distress ventilator 4102 may include operation, or modes of operation, for use outside of an emergency care context. For example, in some embodiments, the respiratory distress ventilator 4102 may include and/or may be coupled with one or more spirometers that can be used for patient respiratory assessment outside of an emergency care situation. For example, such assessment may be performed on patients or individuals that may not be presently receiving mechanical ventilation, may not require mechanical ventilation, and may not be presently experiencing RD. In some embodiments, such assessments may include, for example, assessing and measuring the change in one or more respiratory parameter values (e.g., FEV1 or FVC) before and after some change of circumstances or procedure, such as, for example, bronchodilator treatment, or respiratory parameter and respiratory status assessments on a patient during a routine, scheduled or other doctor's office, or medical facility or hospital. Furthermore, some assessments may be made while the patient is be laying, sitting or standing, and while the patient is not experiencing RD.

In the embodiment depicted in FIG. 1, the respiratory distress ventilator 4102 includes one or more flow sensors 4130, pneumotachometers or pressure sensors, which may be disposed within the patient circuit 4116, or within, elsewhere within, or as part of the respiratory distress ventilator 4102, for sensing signals representative of gas flow within the gas delivery apparatus of the respiratory distress ventilator 4102. In embodiments, the one or more flow sensors 4130 may be coupled with or part of one or more spirometers, for example. In some embodiments, a controller of the respiratory distress ventilator 4102 receives the signals representative of the gas flow. Based at least in part on the signals representative of the gas flow, the controller may generate respiratory parameter data corresponding with at least one respiratory parameter of the patient, such as, for example, Crs, Rrs, VC, FVC, FEV at a timed interval such as FEV1, FEF, or FEF25-75, PEF, PEFR, MVV or others.

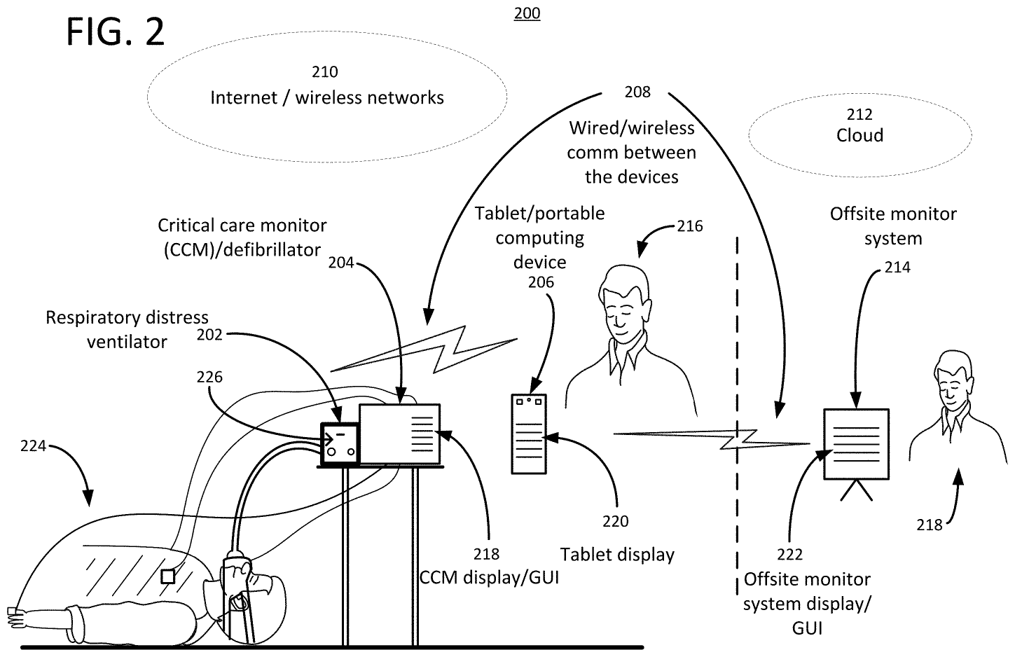

The controller may transmit the respiratory parameter data to be received (directly or via one or more intermediary entities) by, e.g., the tablet 4108, CCM/defibrillator 4104, or elsewhere, such as for use in determining a respiratory status of the patient. However, in some embodiments, the respiratory distress ventilator 4102 may determine, or participate in determining, the respiratory status of the patient. Various examples of embodiments including a respiratory distress ventilator 4102 are described with reference to FIGS. 2-7.

In some embodiments, aspects of a system of devices including a respiratory distress ventilator 4102, such as distribution and integration of roles between the devices as well as aspects of particular devices, may be optimized based on factors that may include a desired degree of portability or ease of operation of the respiratory distress ventilator 4102, for example. Furthermore, aspects of individual devices may be optimized. Such aspects may include, for example, sets of controls, processing and memory aspects, software, algorithms, and CSG related aspects. In some embodiments, various capabilities may be included with the respiratory distress ventilator 4102, or included with one or more other devices or systems, or may be distributed or integrated between them. This may include, for example, processors, memories, controllers, or stored software or algorithms. These capabilities may be used, for example, in processing signals, generating respiratory parameter data or determining a respiratory status of the patient. They may also be used in performing coordination or integration between devices. As such, in this and other ways, the roles and processing capability and components of the respiratory distress ventilator 4102 may be greater or fewer, or determined or allocated, based at least in part on the roles of one or more other devices in an environment. In some embodiments, reducing or limiting the roles, processing capability or components of the respiratory distress ventilator 4102, as well as its included set of controls 4144, can increase or improve the portability or portable practicality of the respiratory distress ventilator 4102, such as by allowing reduced respiratory distress ventilator size, weight, complexity, bulk, footprint, or noise during operation, for example. This, in turn, can allow for various implementations that may be balanced or optimized for particular anticipated or likely scenarios, which can increase positive, potentially life-saving patient outcomes.

In some embodiments, for example, a respiratory distress ventilator may have a size of between 0.125-27 L, such as, e.g., between 5.0-27 L, between 1.0-5.0 L, between 0.5-1.0 L, between 0.15-0.5 L, or between 0.125-0.15 L . Furthermore, in some embodiments, a respiratory distress ventilator may have physical dimensions of between 50×50×50 mm-300×300×300 mm, such as, e.g., between 50×50×50 mm-60×60×60 mm, between 60×60×60 mm-75×75×75 mm, between 75×75×75 mm-100×100×100 mm, between 100×100×100 mm-200×200×200 mm, or between 200×200×200 mm-300×300×300 mm. Furthermore, in some embodiments, a respiratory distress ventilator may have a weight of between 0.2-5.0 kg, such as, e.g., between 1.0-2.0, between 0.3-1.0, or between 0.2-0.3 kg. Furthermore, in some embodiments, a respiratory distress ventilator may have a maximum noise (or noise during a particular operating mode) of 40-70 dB at 1 m, such as, e.g., between 40-50 dB at 1 m, between 50-60 dB at 1 m, or between 60-70 dB at 1 m. Furthermore, in some embodiments, a respiratory distress ventilator may have a battery life of between 2-10 hours, such as, e.g., between 2-4 hours, between 4-6 hours, between 6-8 hours, or between 8-10 hours. In some embodiments, the battery can be charged while the respiratory distress ventilator is operating.

In the embodiment depicted in FIG. 1, each of the respiratory distress ventilator 4102, the CCM/defibrillator 4104 and the tablet 4108 includes a set of one or more controls (which can be physical, display/GUI based, or both). Particularly, as depicted, the respiratory distress ventilator 4102 has a set of included controls 4144, including one or more physical controls 4146 (e.g., one or more physical switches, buttons, knobs, dials, etc.) and one or more GUI/display based controls 4142. However, in some embodiments, the set of included controls 4144 may include only one or more physical controls, only one or more display/GUI based controls, or no controls at all. The CCM/defibrillator 4104 (which may, in some embodiments, be an automated external defibrillator, or AED) has a set of CCM/defibrillator controls 4134, including one or more physical controls 4132 and one or more GUI/display based controls 4110. The tablet 4108 has a set of tablet controls 4112, including one or more physical controls 4138 and one or more GUI/display based controls 4136. In various embodiments, aspects of various sets of controls 4144, 4134, 4112 may be distributed in various ways, and may or may not include similar, identical or overlapping aspects.

In some embodiments, the set of tablet controls 4112 may display data relating to various patient physiological, respiratory and ventilation related parameters, and may include other output or presentation components, such as a speaker and/or a microphone. For example, a microphone could be used to determine the level of ambient noise, which could be used in determining and setting, or using particular settings to achieve, an appropriate volume level or volume control of the respiratory distress ventilator or its components. Furthermore, a combination of a speaker and microphone, used with appropriate software that could be stored on the respiratory distress ventilator, remotely, or both, could be used (such as in combination with, or in some instances instead of, GUI based interaction) in implementing or delivering user interactive CSG or other interaction with the user. For example, this could involve voice input provided by the user via the microphone, such as may include commands, confirmations, questions, responses or answers. It could also include voice output such as may include prompts, confirmations, questions, responses, answers, guidance, instructions or step-by-step instructions delivered to the user using the speaker. In various embodiments, the voice output may be algorithmically determined by the respiratory distress ventilator or an associated remote system, or may be provided by another user or care provider speaking to the user via the speaker, where the other user or care provider uses a microphone of a device of the other user or care provider. Furthermore, the user input could be directed to the respiratory distress ventilator itself, or to another care provider, or both. The other user or care provider could be relatively close to the user, such as in the area or a different portion of the care scene, or could be distant from the user, such as at a remote interface, which could include, for example, a doctor or other care provider at a hospital or medical facility, for example. In some embodiments, for example, the microphone and speaker of the respiratory distress ventilator may allow the user to interact in real time with, or take step by step instructions from, the doctor or other care provider concerning emergency care to the patient, whether relating to ventilation or other care.

Additionally, the set of tablet controls 4112 may include display of integrated data relating to operation of the respiratory distress ventilator 4102 as well as other devices that may also be in use, such as, for example, the CCM/defibrillator 4104. In some embodiments, the set of tablet controls 4112 may allow user interaction, including to obtain or display data, change settings of the respiratory distress ventilator 4102, accept or confirm suggested or recommended settings changes, or view or respond to alarms, alerts, or messages, among other things.

In the embodiment depicted in FIG. 1, any, some or all of the set of included controls 4144 of the respiratory distress ventilator 4102, the set of CCM/defibrillator controls 4134 or the set of tablet controls 4112 may have one or more respiratory aspects. Either or both of the set of CCM/defibrillator controls 4134 and the set of tablet controls 4112 may include remote controls relating to the respiratory distress ventilator 4102, such as may relate to controlling aspects of provided mechanical ventilation, among other things. Furthermore, any of the sets of controls 4144, 4134, 4112 may, for example, include display/GUI aspects relating to respiratory parameter data, respiratory status, or CSG. In some embodiments, the set of included controls 4144 of the respiratory distress ventilator 4102 may be limited, while the set of CCM/defibrillator controls 4134, and/or the set of tablet controls 4112, may be more comprehensive, or otherwise.

Furthermore, in some embodiments, remote control may include or allow relatively sophisticated or complex aspects of mechanical ventilation operation or control, such as may include, for example, CLC such as FiO2 CLC, PEEP CLC or CLC of one or more other ventilation parameters. Also, in some embodiments, the respiratory distress ventilator 4102 may be configured to switch to, or operate using, internal control as a backup or default form of control, such as when a fault mode condition is detected. Examples of fault mode conditions are provided with reference to FIGS. 18-21. The respiratory distress ventilator 4102 may further be configured to detect when a fault mode condition is no longer present, and then switch back to or operate using internal control. In various embodiments, the respiratory distress ventilator 4102 may be remote controlled by the CCM/defibrillator 4104, the tablet 4108, or one or more other devices or systems.

FIGS. 2-7 illustrate additional example environments including a respiratory distress ventilator as well as other devices, systems or monitors, although many other possibilities and combinations may be used. In each of the example environments, in various implementations, any of the depicted devices, systems or monitors may include controls with respiratory aspects and may include remote controls relating to the respiratory distress ventilator. Furthermore, in various implementations, the respiratory distress ventilator may have any of a spectrum of different sets of included controls, from no included controls to a large set of included controls, some examples of which are described further with reference to FIGS. 28A-C. Still further, in various implementations, various sets of remote controls may or may not in whole or in part reflect aspects of any included controls of a respiratory distress ventilator. Additionally, in various implementations, roles or sensing capabilities may be variously included and distributed between devices, systems, monitors or users.

FIG. 2 illustrates an example emergency care environment 200 including a respiratory distress ventilator 202 with a respiratory distress ventilator display/GUI 226, a CCM/defibrillator 204 with a CCM defibrillator display/GUI 218, a tablet 206 (or other portable computing device) with a tablet display/GUI 220, and an offsite monitor system 214, which may include one or more server computers, with an offsite monitor system display/GUI 222. In various embodiments, all or some of the devices or systems 202, 204, 206, 214 may be coupled via wired connection and/or wirelessly via one or more wireless networks 210 which may include the Internet. The tablet 206, or other devices, may be coupled with the remote monitor 214, for example, as aspects of a cloud 212 based computing system or network. A local user 216 is shown, as well as an offsite user 218 at the remote, offsite monitor system 214.

In the embodiment depicted in FIG. 2, local user 216 (or more than one), interacting via the display/GUI 220 of the tablet 206, the display/GUI 218 of the CCM/defibrillator 204, and/or the display/GUI 226 of the respiratory distress ventilator 202, is in communication with the offsite user 218 (or more than one), interacting via the display/GUI 222 of an offsite monitor system 214. In some embodiments, more than one local user may be present and in communication with each other, and the various devices or systems 202, 204, 206, 214 may be in direct or indirect communication, and/or coordination or integration, with each other. The offsite user 218 may be, e.g., a doctor, nurse or other skilled clinician or care provider, such as at, or associated with, a hospital or medical facility, group or entity. In various embodiments, data relating to patient care, which may include respiratory aspects, may be communicated, and may be integrated, between various of the devices or systems 202, 204, 206, 214 with or without user interaction. This may, for example, allow the offsite monitor system 214, and/or the offsite user 218 interacting with and via the offsite monitor system 214, to determine and provide guidance, instruction or CSG to the local user 216 via a local device 202, 204, 206. Furthermore, in some embodiments, any or some combination of the CCM/defibrillator 204, the tablet 206 and the remote monitor system 214 may control, or participate in control, or at certain times or time periods control, aspects of operation of the respiratory distress ventilator 202 in providing mechanical ventilation to the patient 224, such as may include controlling one or more ventilation parameters. This may be done with or without user interaction, or subject to user interaction, where permitted, such as if a user changes, sets or overrides certain parameters.

In some embodiments, the respiratory distress ventilator 202 may obtain and communicate patient respiratory parameter data to one or several of the other devices or systems 204, 206, 214, and/or data may be exchanged between various of the devices or systems 202, 204, 206, 214. One or more of the devices or systems 202, 204, 206, 214, may use the communicated patient respiratory parameter data in determining a patient respiratory status (including associated data). Patient respiratory parameter data and/or respiratory status data may be used, for example, in determining CSG. In some embodiments, however, the respiratory distress ventilator 202 may determine, or participate in determining, and may store, the patient respiratory parameter data and/or the patient respiratory status data, and may then communicate determined data to other devices or systems, for example.

In the environment 200 depicted in FIG. 2, in various embodiments, the respiratory distress ventilator 202 may be capable of remote control by one or more of the CCM/defibrillator 204, the tablet 206 or the remote monitor system 214.

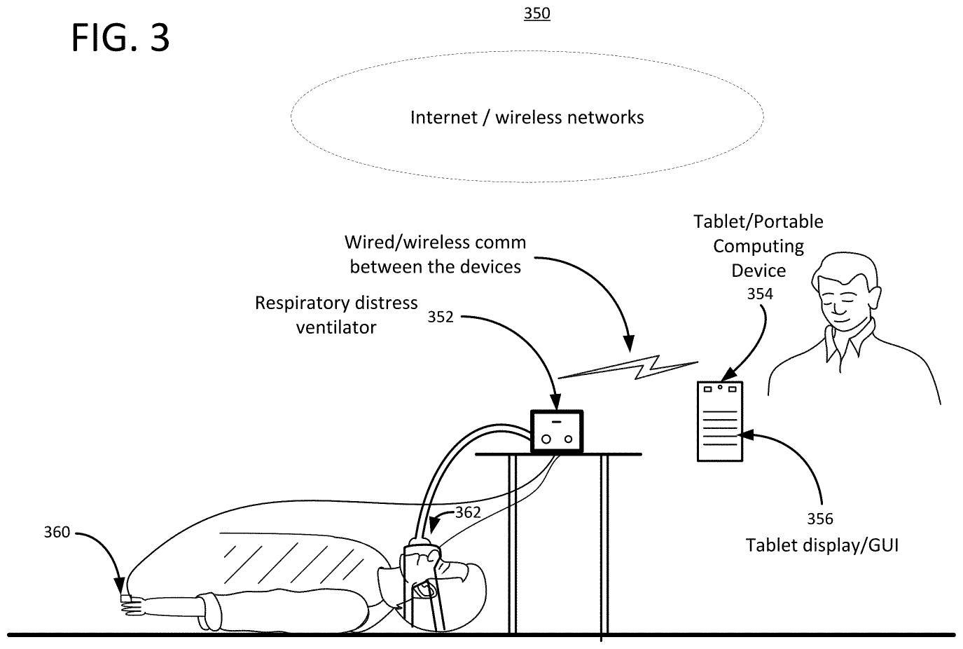

FIG. 3 illustrates an example emergency care environment 350 including a respiratory distress ventilator 352 and a tablet 354 with a tablet display/GUI 356 that may include respiratory aspects (such as may include CSG) and may be capable of remote control of the respiratory distress ventilator 352. In the embodiment depicted in FIG. 3, the respiratory distress ventilator 352 includes a pulse oximeter 360 for measuring patient SpO2, a capnographic sensor 362, which may be used in measuring EtCO2, and may include other sensing components for sensing other patient parameters. In some embodiments, however, some or all sensing components may be included with or distributed between the tablet 354 and/or other devices, for example.

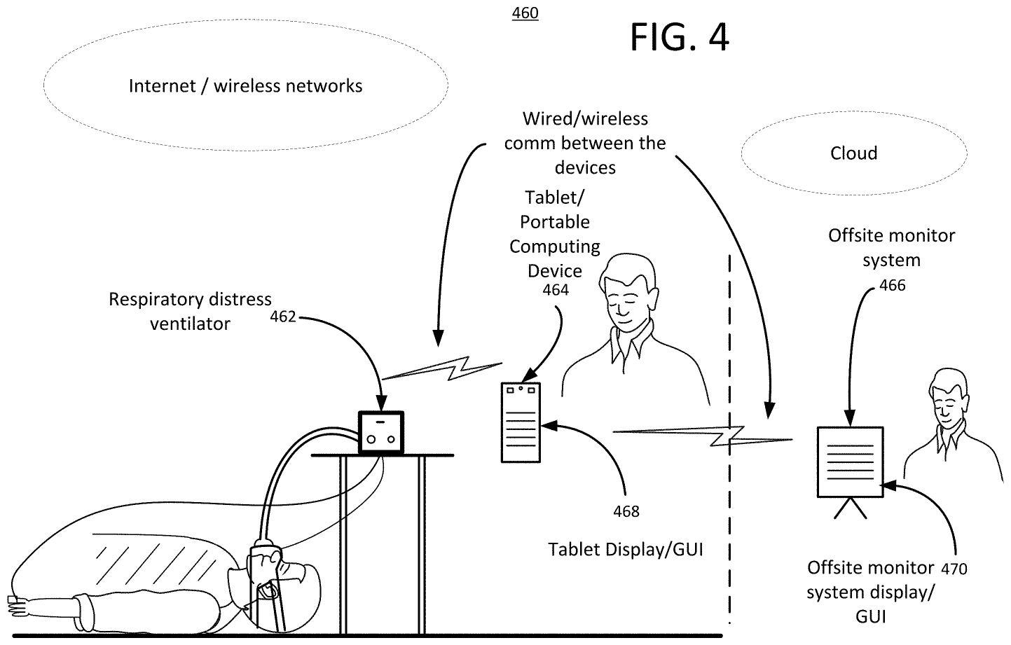

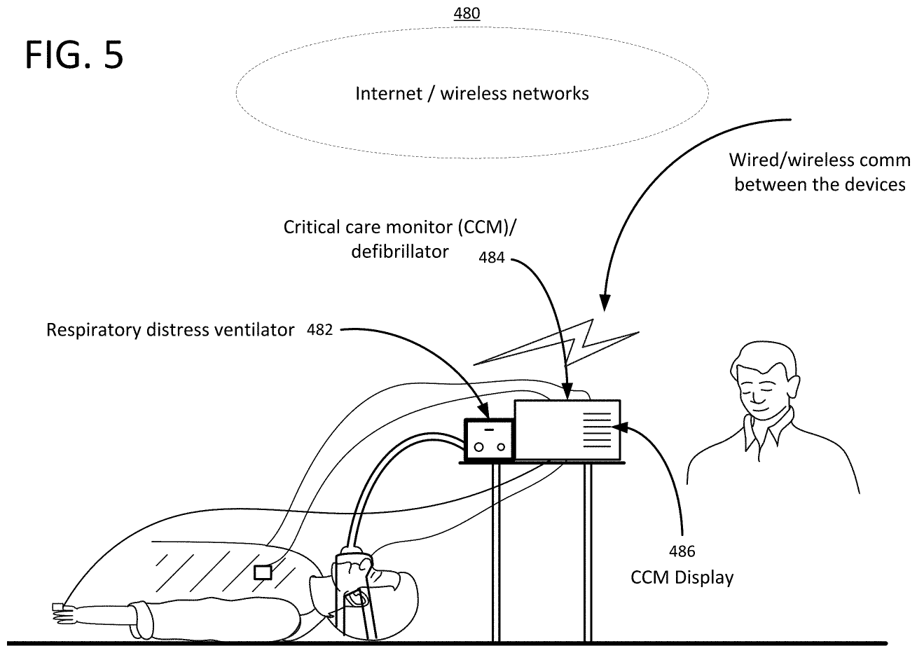

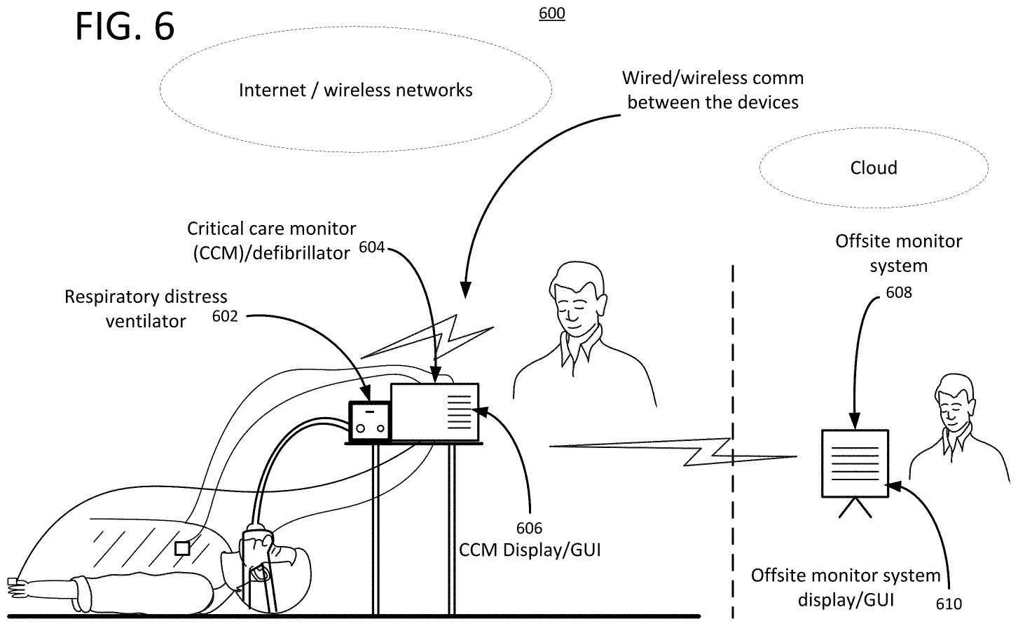

FIGS. 4-7 illustrate schematic diagrams of additional emergency care environments 460, 500, 600, 700 in accordance with various embodiments, including devices or systems that may be communicatively coupled via wired or wireless connection. Particularly, FIG. 4 illustrates an environment 460 including a respiratory distress ventilator 462, a tablet 464 including a display/GUI 468 that may include respiratory aspects or CSG, and an offsite monitor system 466 including a display/GUI 470 that may include respiratory aspects or CSG. FIG. 5 illustrates an environment 480 including a respiratory distress ventilator 482 and a CCM/defibrillator 484 including a display/GUI 486 that may include respiratory aspects or CSG. FIG. 6 illustrates an environment 600 including a respiratory distress ventilator 602, CCM/defibrillator 604 including a display/GUI 606 that may include respiratory aspects or CSG, and an offsite monitor system 608 including a display/GUI 610 that may include respiratory aspects or CSG.

Each of the embodiments described in FIGS. 1-7 can provide various advantages. In some embodiments, a respiratory distress ventilator can be used with a CCM/defibrillator, as shown, for example, in FIGS. 1-2 (also including a tablet or other portable computing device) and FIGS. 5-6 (not including a tablet or other portable computing device). In some implementations, the respiratory distress ventilator can, in some ways, serve or function as an accessory treatment device to the CCM/defibrillator. For example, the CCM/defibrillator may provide sensing, or patient parameter sensing, capability, such as oximetry, capnography, blood pressure monitoring, and/or other sensing capability. Additionally, the CCM/defibrillator (or, in some embodiments, a tablet, or both) may play an important role in providing display/GUI features, which may include CSG. As such, in various implementations that include a CCM/defibrillator, the respiratory distress ventilator may include or provide more limited sensing capability, or display/GUI or CSG capability. As such, in some implementations, the respiratory distress ventilator may be simpler, smaller, lighter, and or more easily, quickly or conveniently portable, and/or its functionality, hardware and software may be less or simpler. For example, in some implementations, a care provider with a respiratory distress ventilator may arrive at a patient scene at which a different care provider (or several) is already providing care with (at least) a CCM/defibrillator, where the respiratory distress ventilator may be able to integrate, providing respiratory monitoring and ventilation capability, even after care and sensing has been initiated using at least the CCM/defibrillator. Alternatively, a care provider with a respiratory distress ventilator may arrive at the patient scene first, and one or more other devices may arrive and be integrated later.

In some embodiments, a respiratory distress ventilator can be used with a tablet or other portable computing device as shown, for example, in FIGS. 1-2 (also including a CCM/defibrillator), and FIGS. 3-4 (not including a CCM/defibrillator). In some implementations, a tablet or other portable computing device can serve as a device with an integrated and comprehensive display/GUI. As such, in some implementations, the respiratory distress ventilator may include less display/GUI and/or CSG features, and/or associated hardware or software, since the tablet or other portable computing device may provide, or to a degree provide, these features. In some embodiments, this can allow the respiratory distress ventilator to be simpler, smaller, lighter and/or more easily, quickly or conveniently portable.

In some embodiments, a remote, offsite care provider, and/or offsite computer system, may be included, such as shown, for example, in FIGS. 2, 4 and 6. In some implementations, the offsite care provider (and/or computer system) can provide additional support (computational or human), or expertise, beyond that which may be available locally. For example, in some embodiments, a more trained care provider or medical expert, such as a medically trained individual, nurse or doctor, may provide CSG, support, guidance, and/or even step by step instructions via a remote interface (and potentially utilizing offsite computing system capabilities, including hardware and software), such as may be delivered to the user of the respiratory distress ventilator, for example, via a display/GUI, and potentially speaker, of a tablet, CCM/defibrillator or respiratory distress ventilator, where the user of the respiratory distress ventilator may interact via the display/GUI and/or potentially a microphone of one of the local devices.

In some implementations, by integrating the role of the offsite care provider and/or offsite computing system, additional or enhanced care may be provided, which could include additional interaction and care enhancements or capabilities. Furthermore, in some embodiments, a video camera and display at the scene of the user of the respiratory distress ventilator or respiratory distress ventilator could additionally enhance this interaction and care capabilities, with the user of the respiratory distress ventilator potentially taking step by step instructions from the offsite care provider, who may also use a video camera and microphone to provide video and audio feed to the user. The offsite care provider may be informed in part by communicated real-time video and audio data, feed or stream, which could include a view of and/or sounds of the patient as well as the user of the respiratory distress ventilator (and/or one or more other local care providers and/or devices). In some embodiments, this could include, for example, 3D video or augmented or enhanced reality presentation, to either or both of the user of the respiratory distress ventilator or portable ventilator and the offsite care provider. For example, in some embodiments, either or both may use or wear one or more augmented or enhanced reality devices in this regard, such as a watch, head-worn or body-worn device, with video and audio capability. In various embodiments, this could provide hands-free use, or could even include motion or other sensors to track and display movements of the user and/or the offsite care provider.

In some embodiments including a respiratory distress ventilator, a CCM/defibrillator may not be included, such as shown, for example, in FIGS. 3 (including a tablet or other portable computing device but not an offsite monitoring system) and 4 (including a tablet or other portable computing device and an offsite monitoring system), and, in some embodiments, a respiratory distress ventilator may be used without a CCM/defibrillator, tablet or portable computing device or remote monitoring system. In some implementations, by not including a CCM/defibrillator, the overall system used in monitoring and treatment of the patient may be more compact, simper, smaller, lighter, and/or more easily, quickly or conveniently portable. However, =in many embodiments and implementations, the flexible, integrated, quickly adaptable nature of the system can allow for changes to included or excluded devices or systems, while still maintaining optimal patient care with presently available devices and systems. This can include, for example, adding, removing or shifting functionality or roles provided by currently available remaining devices or systems (or users), such as when a device or system (or user) is removed (or otherwise unavailable) or added (or otherwise available) to the overall system.

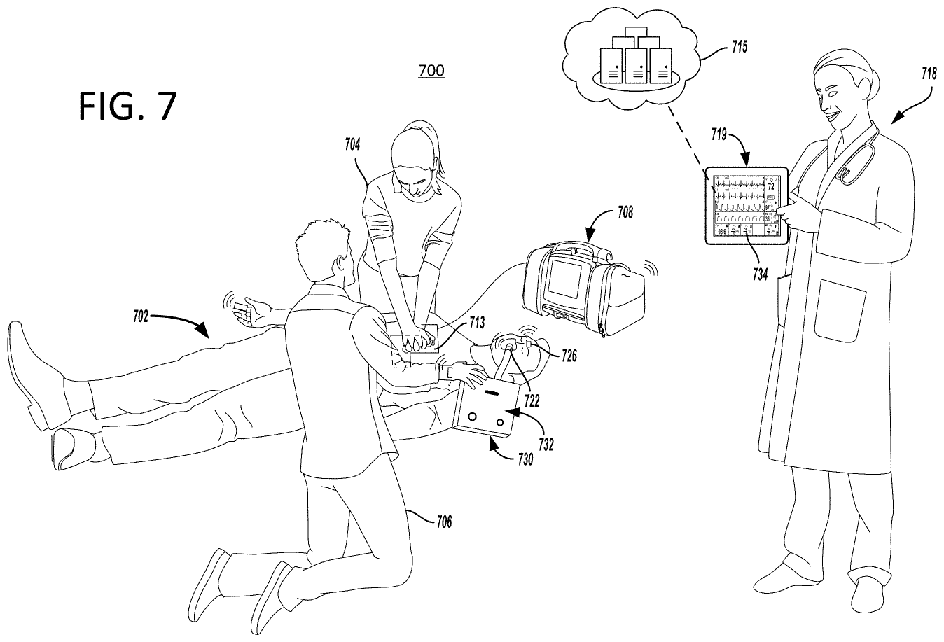

FIG. 7 illustrates an example emergency care environment 700 including a respiratory distress ventilator 730, a defibrillator/critical care monitor (CCM) 708 and one or more companion devices, such as companion tablet 719. In the embodiment depicted, the respiratory distress ventilator 730 has a limited set of included controls 732 (physical, display/GUI based, or both). However, the tablet 719 has a display/GUI 734 that includes a comprehensive set of remote controls associated with the respiratory distress ventilator 730 and its operation, which may include respiratory aspects or CSG.

In some embodiments, sensor data obtained by the one or more sensors (e.g., 722, 726) associated with one or more of the devices 708, 719, 730 can be transmitted to a companion device (e.g., tablet 719) for display within one or more user interface views. For example, the companion device 719 (or one or more other of the devices 732, 708) may display, for example, among other things, some or all of: ECG and defibrillator shock data, airway pressure (Paw), plateau pressure (Pplat), peak inspiratory pressure (PIP), SpO2, FiO2, PEEP, FVC, FEV1, PEF/PEFR), Rrs, Crs, inspired and expired tidal volume, Ve, EtCO2, volume of exhaled carbon dioxide (VCO2), breaths per minute (BPM) and heart rate (HR).

In addition, the tablet 719 can transmit instruction or command signals to the respective medical treatment device 708, 730. The transmitted instruction signals can provide information, initiate one or more functional operations, or set or change operational parameters or modes of the respective medical treatment device 708, 730.

In some implementations, patient treatment information associated with both medical treatment devices 708, 730 (and potentially others) and/or other data, such as caregiver performance data, respiratory parameter data, or patient respiratory status data, can be displayed at the companion device 719. In some examples, supervisor 718 can monitor patient treatment information received from the defibrillator 708 and the respiratory distress ventilator 730 at the companion device 719. In some examples, patient treatment information can be displayed within one or more selectable views at the companion device 719 or within a single user interface view. In addition, in some embodiments, the user interface views at the companion device 719 can include user inputs that allow the supervisor 718 to transmit instruction signals to provide data or issue commands to control one or more functional operations of the medical treatment devices 708, 730. In some embodiments, providing the ability for a single caregiver to view case information from multiple medical treatment devices 708, 730 provides a technical solution to a clinical problem in that a caregiver and/or supervisor using the companion device 719 can provide enhanced coaching and support to other rescuers 704, 706. Further, in certain emergency care situations where patient access or rescue personnel are limited, providing a single caregiver with the ability to monitor case information simultaneously from multiple medical treatment devices 708, 730 and transmit instruction signals to control operation of the medical treatment devices 708, 730 enables patients to receive advanced medical care in remote or non-traditional care environments. However, some embodiments include various roles for various users.

In some embodiments, a local wireless communication channel can be established among two or more of the devices at the emergency care scene 700 to enable data to be securely and accurately shared among the devices and systems. For instance, health data about the patient 702, data indicative of treatment delivered to the patient 702, or other types of data can be exchanged over a wireless communication channel, potentially including one or more remote, offsite systems 715. This can help enable treatment by multiple rescuers to be coordinated or integrated in an efficient and accurate manner. In some examples, wired and/or wireless communication channels are established among only some of the devices involved in treatment of the patient 702 (e.g., between two of the devices). In some examples, a wireless communication channel is established among all of the devices involved in treatment of the patient 702.

FIG. 8 illustrates an example modular system 750 in accordance with some embodiments, including a respiratory distress ventilator 752, a CCM/defibrillator 754 (shown from two different angles) and a tablet 756 or other portable computing device. Such a system may provide various advantages. For example, in some implementations, users with different devices may arrive at a patient scene at different times. A modular system, such as that depicted in FIG. 8, may allow advantages such as faster or staged set up or operation and/or multifaceted integration of devices. This can save time as well as optimize efficiency and operation of the system as a whole, which can be critical or life-saving in an emergency care context.

In various embodiments, in the modular system 750, one or several of the depicted devices 752, 754, 756 may not be included, or any of various other devices may be included, such as one or more medical devices, monitoring devices or portable computing devices. In some embodiments, two or more devices in a local environment (such as any or all of the depicted devices 752, 754, 756 and/or other devices that may be included) may be adapted for, and capable of, being joined or attached (fixedly or movably). For example, one or more devices may be capable of being joined or attached to, and disjoined or detached from, one or more other devices. In some embodiments, one device (or one of several different devices) may serve as a device to which several other devices may be joined or attached. Furthermore, in some embodiments, one or more docks or docking stations may be included, to which each of multiple devices (e.g., some or all of the devices 752, 754, 756 or other devices) may be docked (which may include being joined or attached to the dock or docking station). Still further, a dock or docking station may be included to which multiple devices can connect via wired or wireless connection without joining or attaching to the dock or docking station, such as via wired connection using one or more plug-in cables, for example.

In various embodiments, any or each of the devices, and any included dock or docking station, may include physical, mechanical or electrical components that may facilitate joining, attachment or docking, and may facilitate electrical connection, communication and/or powering. Such powering may include partial, optional or selective powering, as well as powering via wired or wireless connection, such as between devices, between docked devices, or between a dock or docking station and one or more docked devices. Still further, in various embodiments, joined, attached or docked devices may be configured to automatically communicate, authenticate, coordinate or integrate operationally, potentially including aspects relating to display aspects (which may include CSG), sensing capabilities, processing, software, and powering, among other things, which may be enabled or facilitated by the joining, attachment or docking.

In the embodiment shown, the CCM/defibrillator 754 includes physical or mechanical coupling components, such as guide or attachment rails 758 by which the respiratory distress ventilator 752 and the tablet 756 may be coupled to the CCM/defibrillator 754, such as, for example, by being slid into the rails, or in other ways. Although not shown, in some embodiments, the respiratory distress ventilator 752, the CCM/defibrillator 754 and/or the tablet 756 may also have one or more components to facilitate joining or attachment, such as complimentary guide or attachment grooves, for example. However, any of various joining or attachment designs, components, mechanisms or methods may be used in various embodiments. Although, in the embodiment shown, the CCM/defibrillator 754 serves as the device to which the other devices 752, 756 join or attach, in other embodiments, either of the respiratory distress ventilator 752 or the tablet 756 or other portable computing device, or another device may serve that role, and/or a dock or docking station may be used.

As described with reference to FIGS. 1-7, in various embodiments, the respiratory distress ventilator 752 may be capable of being remote controlled by one or more other devices, such as the CCM/defibrillator 754 or the tablet 756. Furthermore, some or each of the respiratory distress ventilator 752, the CCM/defibrillator 754 and the tablet 756 may include displays/GUIs that include respiratory aspects or CSG.

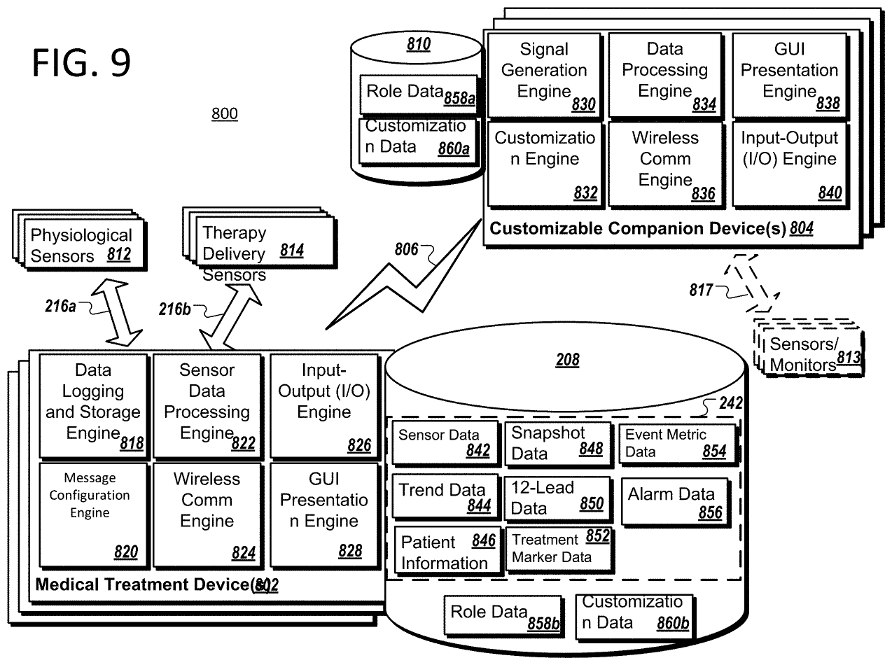

FIG. 9 depicts an example environment 800 for customizing display of case information at, e.g., a companion device (e.g., tablet or other portable computing device as depicted in FIGS. 1-8), including a medical treatment system with a set of devices for providing treatment to a patient during a medical event. The environment 800 may include one or more medical treatment devices 802 (e.g., respiratory distress ventilator, or CCM/defibrillator, as depicted in FIGS. 1-8, or others) and one or more companion devices 804 communicatively coupled to the medical treatment device 802 via communication link 806. In some embodiments, the companion device 804, or another device, may be capable of remote control of a respiratory distress ventilator. The devices 802, 804, in the illustrated example, of the medical treatment system may be co-located at a treatment delivery site, such as a hospital, medical clinic, or ambulance. In other implementations, one or more of companion devices 804 may be added to the system at some point during delivery of a medical therapy or subsequent to delivery of therapy. Conversely, at least one companion device 804 may be removed from co-location during or subsequent to delivery of therapy. The devices may be configured for data communication in a wired or wireless manner for transferring information between certain devices 802, 804 of the system during and/or subsequent to delivery of therapy.

In some implementations, the wireless communication link 806 for connecting the medical treatment device 802 and the companion device 804, in some examples, may be a Wi-Fi network, other short-range wireless communication network or near field communication (NFC) network, local area network (LAN), wide area network (WAN), or the Internet. In other examples, the devices 802, 804 can be configured to communicate over longer communications ranges such as over a cellular communication network. In some implementations, the medical treatment device 802 may function as a wireless access point to provide a direct wireless connection with the companion device 804. In other examples, the wireless communication link 806 can be provided via Bluetooth personal area network.

In some embodiments, different devices 802, 804 may be configured to communicate with one another over different types of communication links 806. In some implementations, the devices 802, 804 can be configured to transmit data via a short-range wireless communication transmitter, e.g. a Bluetooth beacon, to a receiver. In one example, a first companion device 804 may communicate with the medical treatment device 802 via a Wi-Fi communication link while a second companion device 804 may communicate with the medical treatment device via a Bluetooth communication link. In some implementations, a companion device 804 can connect to the medical treatment device 802 via the wireless communication link without having to physically access or interact with the medical treatment device 802. In some examples, transport layer security (TLS) is used at an application level to provide a secure (encrypted) connection between the devices 802, 804. As a second layer of protection, encrypted Wi-Fi or encrypted Bluetooth can be used at a physical layer.

In some implementations, when the wireless communication link 806 is a cellular communication link, the functionality of the medical treatment device 802 can be extended to clinicians who are off-scene and/or performing remote telemedicine. For example, when EMS are transporting a patient to the hospital in an ambulance, a medical team awaiting the arrival of the patient to the hospital can stream real-time case information at a companion device 804 at the hospital via cellular link. In some examples, the wireless communication link 806 can include combinations of multiple wireless communication networks based on proximity of the medical treatment device 802 to the companion device 804.

In some implementations, each of the medical treatment device 802 and the companion device 804 includes a respective wireless communication engine 824, 836 for enabling wireless communication between the devices 802, 804 via the wireless communication link 806. For example, the wireless communication engine 824 of the medical treatment device 802 can be configured to transmit messages generated by message configuration engine 820 to the companion device 804. Wireless communication engine 836 of the companion device 804 can be configured to transmit instruction signals generated by signal generation engine 830, for some examples, such instruction signals may be for controlling one or more functional operations of the medical treatment device 802. In some examples, the wireless communication engines 824, 836 are configured to apply encryption protocols to outgoing signals being transmitted to the other device 804, 802. Similarly, the wireless communication engines 824, 836 can decrypt incoming signals from the other device 804, 802.

In certain embodiments, the wireless communication engine 824 of the medical treatment device 802 can be configured to detect that a respective companion device 804 is within communication range and in response, initiates one or more actions to connect to the companion device 804 via the wireless communication link 806. In some implementations, a companion device 804 that is pairable with the medical treatment device 802 can be preconfigured to automatically connect to the medical treatment device 802 via the wireless communication link 806 when within communication range, without having to discriminate between other devices that happen to be within range and/or negotiate a wireless communication connection. Further, rather than requiring a user to potentially spend significant amounts of time in manually configuring the system of each companion device 804 to connect to the medical treatment device 802, or accessing a screen to view and then select from possible device connections, companion devices 804 located at the emergency scene may be pre-configured to dynamically join and/or leave the secure network or pairing with the medical treatment device 802, for example, automatically and/or with one or more simple actions (e.g., switch actuation, pressing a button, near field communication connection, radio frequency, location/proximity recognition, gestural code, tap/bump recognition, motion-activated, sound/vibration, voice command/recognition, amongst others) and/or merely by being in close physical proximity to one another such as by a Bluetooth proximity connection. In some examples, the companion device 804 may be pre-configured for pairing to other medical treatment devices, and those preconfigured networks can also be displayed upon selecting an icon on a display.

Once such connection via the wireless communication link 806 is made, despite the presence of numerous other devices located nearby, patient information (e.g., physiological data, patient history, rescue info) can be sent back and forth between the connected devices 802, 804 in a reliable and secure manner (e.g., according to HIPAA standards, 802.11i protocols) using any suitable type of communication. Companion devices 804 that are correctly paired with their respective medical treatment devices 802 can help avoid risk of erroneous patient information to be transmitted between medical devices, which could be detrimental to patient outcomes. In some embodiments, to maintain accurate and secure communications, the proximity-based interaction may invoke an authentication protocol, such as the use of encrypted keys, vector initialization, hash encryption, digital certificates, etc., ensuring no drops and/or leakage of data transfer between devices. Additionally, the wireless communication engine 824 of the medical treatment device 802 can be configured to simultaneously cause transmission of real-time streaming data to multiple companion devices 804 via separate wireless communication links 806 for each companion device 804.

In some implementations, a number of additional security-oriented design aspects can also be implemented for the medical treatment system to ensure that data exchanged between the medical treatment device 802 and companion device 804 remains secure. For example, the medical treatment device 802 and/or the companion device 804 can use certificate-based authentication to ensure the authenticity of the respective paired device. In some examples, upon initial connection and setup, the devices 804 can execute an association process to tie a particular companion device 804 to a single medical treatment device 802 so that only the companion device 804 (and any other similarly paired companion devices) can interoperate with the medical treatment device 802. In some embodiments, any Representational State Transfer (REST) or Web Socket communications may require an authenticated connection to enable data exchange between the devices 802, 804. The medical treatment device 802, in some examples, prohibits connection to open Wi-Fi communication links and may only connect to manually defined (e.g., supervisor-defined) Wi-Fi networks. As an additional security measure, when the wireless communication link 806 is a Bluetooth connection, the devices are paired during initial setup when initial connection settings are configured. Further, the data and computer architecture of the medical treatment device 802 can be designed for additional security, which can include separating communications and clinical control onto separate microprocessors.

In some examples, when there are more than one companion devices 804 paired with the medical treatment device 802, one of the companion devices 804 may be designated in advance as the primary companion device. The primary companion device may be so designated by the medical treatment device 802 during device setup, pairing and provisioning by receiving and storing an encrypted token from the medical treatment device 802. The encrypted token may be sent with every instruction from the primary companion device 804 to the medical treatment device 802.

In some implementations, the companion device 804 can display status information for the medical treatment device 802 at one or more of display views of the companion device 804. For example, a device view can include a status bar that displays an amount of battery life, connection status, and a unique name for the medical treatment device 802. Additionally, in some examples, a status bar can include a verification input that allows the companion device 804 to cause an indicator to flash at the medical treatment device 802 to confirm that the companion device 804 is connected to the correct medical treatment device 802 at time of use. For example, one or more display views of the companion device 804 can include a paired device verification input that allows a companion device user to verify which medical treatment device 802 the companion device 804 is connected to. In some examples where multiple medical events are occurring at the same time, such as in a trauma unit of a hospital or on a scene of a mass casualty, there may be multiple rescue teams operating multiple medical treatment devices within close proximity of one another. In such situations, it may be easy to mix up companion devices that are paired to respective medical treatment devices. Therefore, in some implementations, when the verification input is selected, the companion device 804 may generate an instruction signal that causes the connected medical treatment device 802 to generate an indication of being paired with the companion device 804. In some implementations, upon receiving a verification signal from the companion device 804, the medical treatment device 802 may output a visual and/or audible indication of being connected to the companion device 804. In some examples, the indication can be a flashing light and/or a tonal sound pulse.

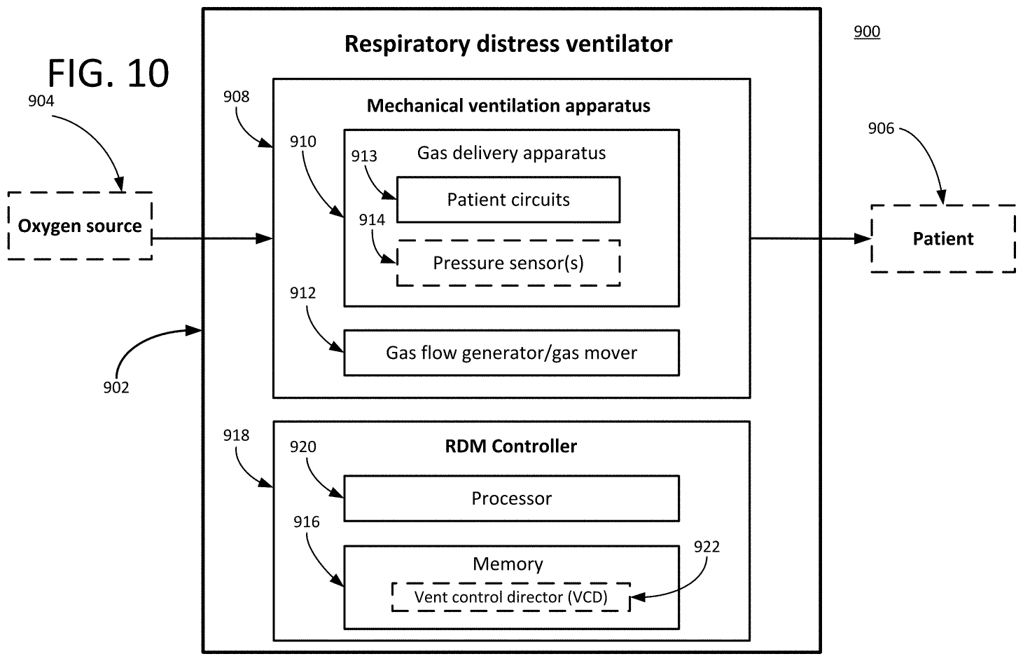

FIG. 10 illustrates an example respiratory distress ventilator 902, in accordance with some embodiments, capable of providing ventilation, and potentially other interventions or treatments, to a patient 906. The respiratory distress ventilator 902 includes a mechanical ventilation apparatus 908 and at least one controller 918. The controller 918 includes at least one processor 920 and at least one memory 916 for storing data, and may also include software that may be stored in the at least one memory 916, such as may include, or may include aspects of, a ventilation control director (VCD) 922, various examples of which are described, for example, with reference to FIGS. 11-25. The respiratory distress ventilator 902 may include or be connected to an oxygen source 904.

In some embodiments, the respiratory distress ventilator 902 is capable of sensing signals representative of gas flow that can be used in determining at least one patient respiratory parameter, such as may include use or one or more pressure sensors 914, flow sensors, pneumotachometers or spirometers, that may, in some embodiments, be included as part of, or within or partially within, the mechanical ventilation apparatus 908, or may be separate but communicatively coupled by wired or wireless connection. In various embodiments, one or more spirometers may be included within the respiratory distress ventilator 902 and/or may be coupled (physically and/or communicatively by wired or wireless connection) with the respiratory distress ventilator 902. Although depicted in a separate box from the patient circuits 913, it is to be understood that the pressure sensors 914 and/or other components such as flow sensors, pneumotachometers or spirometers, may be included within or partially within the patient circuits 913, such as a patient inspiratory circuit and/or a patient expiratory circuit. In some embodiments, the pressure sensor(s) 914 and other associated components may be used in sensing or obtaining respiratory parameter data, which respiratory parameter data may be used in determining or generating a respiratory status (which can include associated data). The respiratory parameter data and respiratory status data may also be used in connection with control of operation of the respiratory distress ventilator 902, such as in control of mechanical ventilation providing by the respiratory distress ventilator 902, whether such control is internally provided by the respiratory distress ventilator 902, remotely provided, or with aspects of both. Furthermore, in some embodiments, the respiratory parameter data and respiratory status may be used in determining or generating CSG, examples of which are described further with reference to, e.g., FIGS. 30-35.

FIG. 11 illustrates an example of an emergency care setting 1000 in accordance with some embodiments. The setting 1000 includes a respiratory distress ventilator 1002 used in providing mechanical ventilation to a patient 1012. The respiratory distress ventilator 1002 may be controlled by a remote control source 1022 including with regard to providing mechanical ventilation. The remote control source 1022 has a set of controls 1014 that includes remote controls for remote control of the respiratory distress ventilator 1002. The respiratory distress ventilator 1002 may have a set of included controls 1010, which may, in some embodiments, be more limited than the remote controls 1014 of the remote control source 1022. In the implementation depicted in FIG. 11, the set of respiratory distress ventilator included controls 1010 includes a power switch and/or indicator 1032 and a communication (or, in some embodiments, remote control) indicator 1030 that may indicate whether communication currently exists between the respiratory distress ventilator 1002 and the remote control source 1022 (or whether the respiratory distress ventilator 1002 is currently being controlled by the remote control source 1022). Some examples of different sets of respiratory distress ventilator included controls are described with reference to, e.g., FIGS. 28A-C.

As depicted, the respiratory distress ventilator 1002 may be remote controlled by the remote control source 1022 via remote control signals 1020, which can include request/command signals, communicated by the remote control source 1022 to the respiratory distress ventilator 1002 (whether directly or via one or more intermediary entities), via wired and/or wireless communication. The remote control signals 1020 are received by the respiratory distress ventilator controller 1006. The respiratory distress ventilator controller 1006 then internally sends control signals 1020 such that the respiratory distress ventilator mechanical ventilation system 1004 is controlled in accordance with the remote control signals 1020. In particular, in the embodiment depicted, a software based ventilation control director (VCD) 1008 is used in implementing control of the mechanical ventilation system 1004 in accordance with the remote control signals 120.

As depicted, the remote control source display/GUI 1014 includes CSG 1018 that may be user interactive and may be used in connection with operation of the respiratory distress ventilator 1002. Further description of examples of CSG are described further with reference to FIGS. 30-35. In some embodiments, respiratory parameter data obtained by the respiratory distress ventilator 1002 is sent to the remote control source 1022, and used in assessing or determining a respiratory status of the patient and in determining CSG.

In the embodiment depicted, control by the remote control source 1022 includes CLC 1024 of any of various types, in connection with provided ventilation, examples of which are provided with reference to FIG. 29. In various embodiments, sensor data used in CLC may be acquired by the remote control source 1022 from various different combinations of one or more devices or systems included in the environment 1000 (locally or offsite). Although, in FIG. 11 and various other figures, CLC 1024 may or may not be depicted, in various implementations and embodiments, CLC 1024 may or may not be included, or may be implemented by any of various depicted devices.

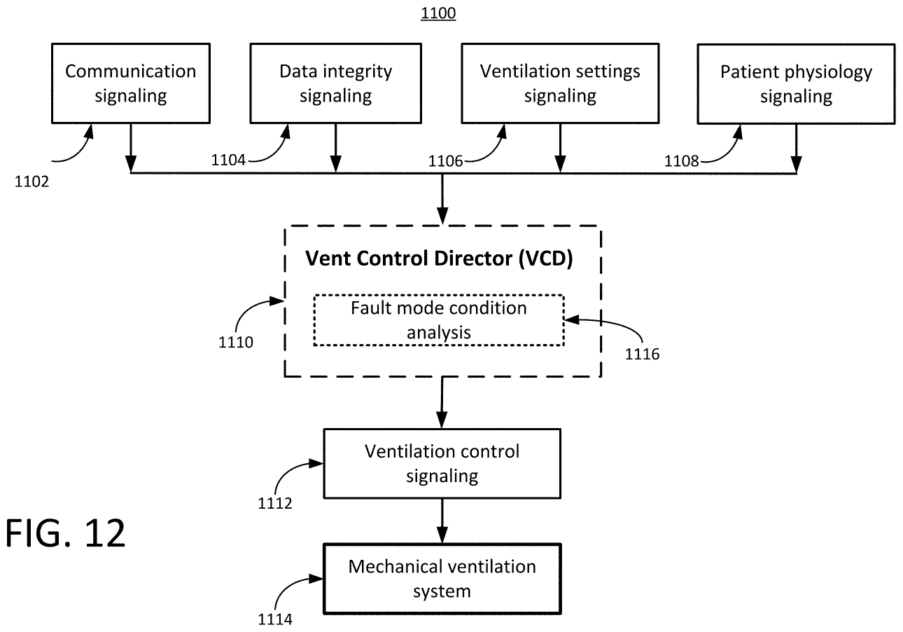

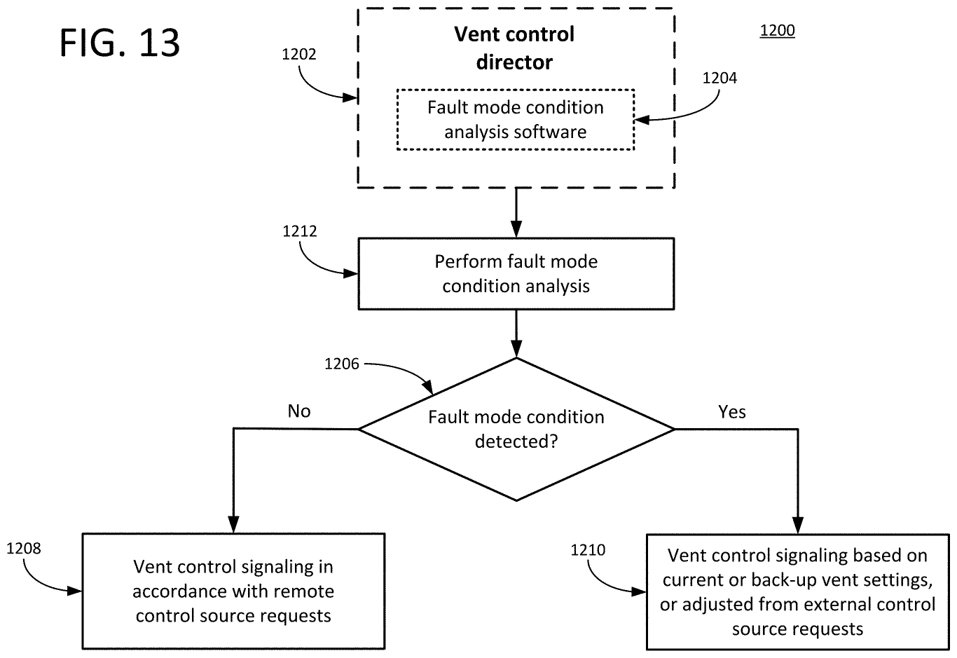

FIG. 12 illustrates examples of signaling 1100 in connection with a VCD 1110 of a respiratory distress ventilator, utilizing fault mode condition analysis 1116, in accordance with some embodiments, detailed examples of which are described with reference to, e.g., FIGS. 13-16. As depicted in FIG. 12, the signaling 1100 includes communication signaling 1102, data integrity signaling 1104, ventilation settings signaling 1106 and patient physiology signaling 1108. However, in various embodiments, other types or categories of signaling may be included.

Communication signaling 1102 may relate, for example, to communication, or presence/existence, degree or speed of communication, between the respiratory distress ventilator and a remote control source. Communication signaling 1102 may also include data used by the VCD to determine whether a remote source is authorized or properly configured for remote control of the respiratory distress ventilator. Communication signaling 1104 may be sent, for example, by a remote control source, and may or may not be sent in response to a received request or other communication by a respiratory distress ventilator.

Data integrity signaling 1104 may relate, for example, to the integrity of data, such as control data, received (directly or via one or more intermediary entities), by the respiratory distress ventilator from the remote control source, such as may relate to, e.g., whether the data is non-corrupt, valid, formatted correctly, bounded correctly or structured correctly. Data integrity signaling 1104 may be sent, for example, by a remote control source, and may or may not be sent in response to a received request/command or other communication by the respiratory distress ventilator.

Ventilation settings signaling 1106 may relate, for example, to particular ventilation parameter settings relating to aspects of mechanical ventilation provided to the patient. Ventilation settings signaling 1106 may be sent, for example, by a remote control source, such as in association with one or more requests, and may or may not be sent in response to a received request/command or other communication by the respiratory distress ventilator.

Patient physiology signaling 1108 may relate, for example, to patient physiological parameters as sensed by sensing aspects of devices or systems, which may include respiratory parameter signals as well as other patient physiological parameter signals. Patient physiology signaling 1108 may or may not be sent in response to a received request or other communication by the respiratory distress ventilator. Furthermore, in various environments and implementations, various types of patient physiology signaling may be sent by or obtained from one or more devices, systems or components in the environment, such as, e.g., a CCM/defibrillator, tablet, other device, or the respiratory distress ventilator, some or any of which devices may, in various embodiments, include various patient parameter sensing capabilities.

The signaling 1102, 1104, 1106, 1108 is received by the VCD 1110. The VCD 1110 performs fault mode condition analysis 1116 based at least in part on at least some of the received signaling 1102, 1104, 1106, 1108. The VCD 1110 sends ventilation control signaling 1112 to a mechanical ventilation system 1114 (or apparatus) of the respiratory distress ventilator based at least in part on the performed fault mode condition analysis 1116.

FIG. 13 illustrates a method 1200 relating to fault mode condition analysis implemented by a VCD 1202 of a respiratory distress ventilator, in accordance with some embodiments. The VCD 1202, using fault mode condition analysis software 1204, performs fault mode condition analysis 1212 in connection with signaling, such as may include, among other things, the signaling 1102, 1104, 1106, 1108 described with reference to FIG. 12.

At step 1206, the VCD 1202 queries whether, based on the fault mode condition analysis 1212, a fault mode condition is detected. If not, then, as represented by box 1208, the VCD 1202 internally sends ventilation control signals to the mechanical ventilation apparatus of the respiratory distress ventilator in accordance with remote control source requests/commands.

However, if the VCD 1202 does detect a fault mode condition, then the VCD 1202 internally controls the mechanical ventilation apparatus of the respiratory distress ventilator, such as based on one or more back-up, default, currently in use, most recent or last received valid settings values, or by settings values that are determined based on adjustment of at least one requested or received setting value.

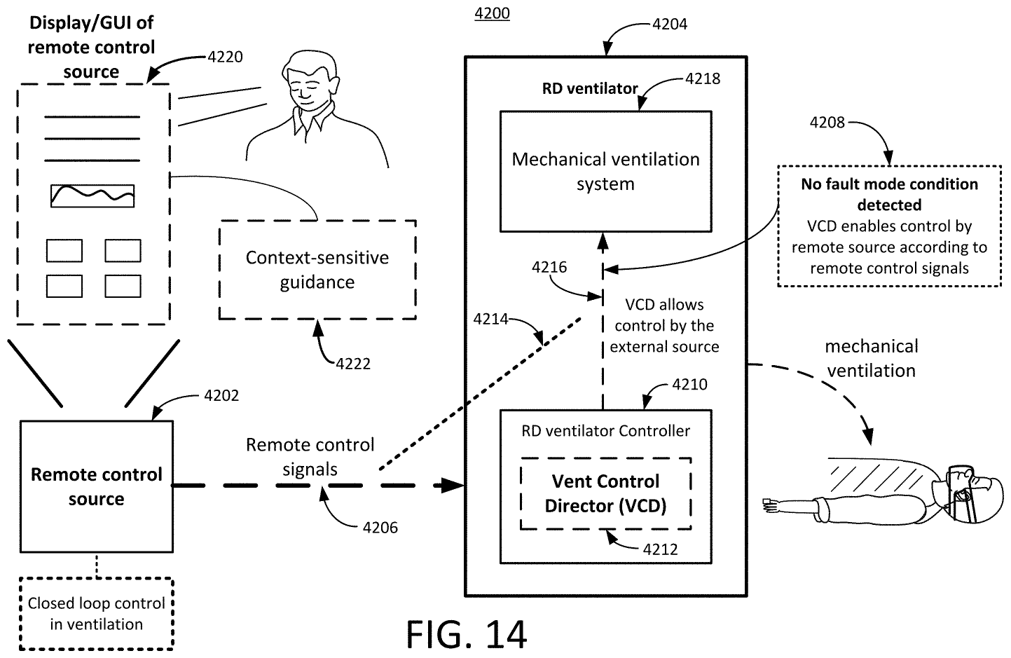

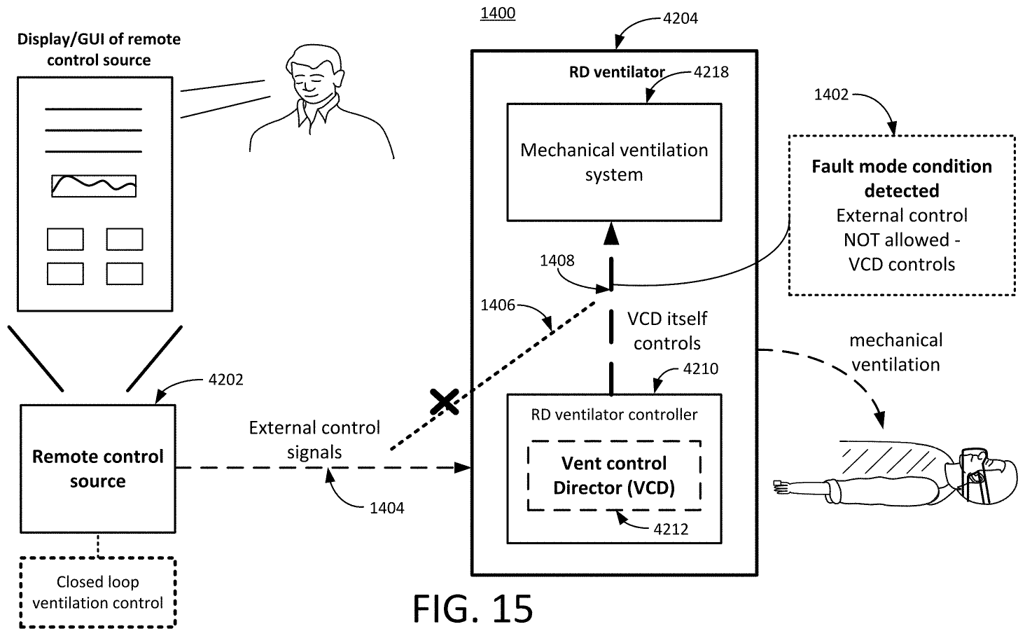

FIGS. 14 and 15 illustrate examples of emergency care environments 1300, 1400 in accordance with some embodiments, including a VCD of a respiratory distress ventilator controller allowing control of respiratory distress ventilator mechanical ventilation by a remote control source. FIGS. 14 and 15 illustrate conditions that may exist at each of two different times, for example.

In the environment 4200 depicted in FIG. 14, at a first current time, a remote control source 4202 sends remote control signals 4206 to be received by the VCD 4212 of the respiratory distress ventilator controller 4210. As indicated by box 4208, at the current time, the VCD 4212 detects that no fault mode condition exists. Accordingly, as conceptually indicated by broken line 4214, the VCD 4212 allows control of the mechanical ventilation system 4218 of the respiratory distress ventilator 4204 by the remote control source 4202 in accordance with the received remote control signals 4206, via internal signaling 4216 from the respiratory distress ventilator controller 4210 to the mechanical ventilation system 4218 of the respiratory distress ventilator 4204 in accordance with the received remote control signals 4206. The display/GUI 4220 of the remote control source 4202 may include respiratory aspects as well as be used in providing CSG 4222.

In the environment 1400 depicted in FIG. 15, at a second current time, the remote control source 4202 sends remote control signals 1404 to be received by the VCD 4212 of the respiratory distress ventilator controller 4210. As indicated by box 1402, at the second time, the VCD 4212 detects that a fault mode condition (or more than one) does exist. Accordingly, as conceptually indicated by crossed out broken line 1406, the VCD 4212 does not allow control of the mechanical ventilation system 4218 of the respiratory distress ventilator 4204 by the remote control source 4202 in accordance with the received remote control signals 1404. Instead, the VCD 4212 controls the mechanical ventilation system 4218 of the respiratory distress ventilator 4204, via internal signaling 1408 from the respiratory distress ventilator controller 4210 to the mechanical ventilation system 4218 of the respiratory distress ventilator 4204 in accordance with control determined by the VCD 4212, such as based on one or more back-up, default, currently in use, most recent or last received valid settings values, or by settings values that are determined based on adjustment of requested or received settings values.

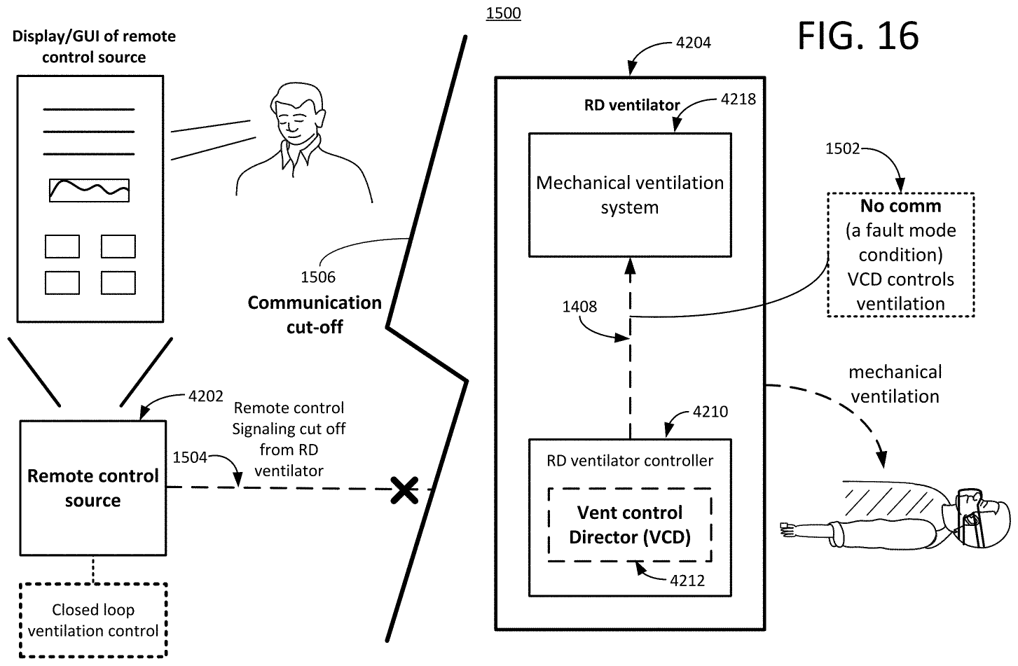

FIG. 16 illustrates an example of an emergency care setting 1500 in accordance with some embodiments, which provides one example of the setting 1400 illustrated in FIG. 15. As indicated by box 1502, at the current time, the VCD 4212 detects that a particular type of fault mode condition exists, which is that no communication or communication capability is detected between the remote control source 4202 and the respiratory distress ventilator controller 4210. As conceptually indicated by bent line 1506, communication between the remote control source 4202 and the respiratory distress ventilator controller is currently cut off or otherwise not present. As such, as conceptually indicated by crossed out broken line 1504, the VCD 4212 does not receive control signals from the remote control source and allow control accordingly. Instead, the VCD 4210 controls the mechanical ventilation system 4218 of the respiratory distress ventilator 4204, via internal signaling 1408 from the respiratory distress ventilator controller 4210 to the mechanical ventilation system 4218 of the respiratory distress ventilator in accordance with control determined by the VCD 4212, such as based on one or more back-up, default, currently in use, most recent or last received valid settings values, or by settings values that are determined based on adjustment of requested or received settings values.

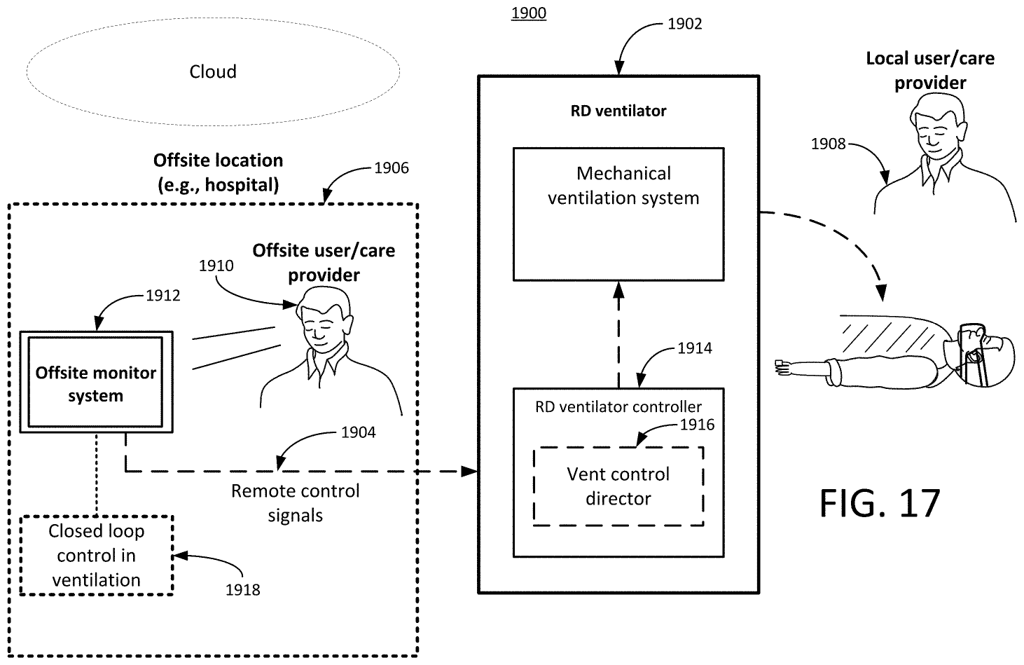

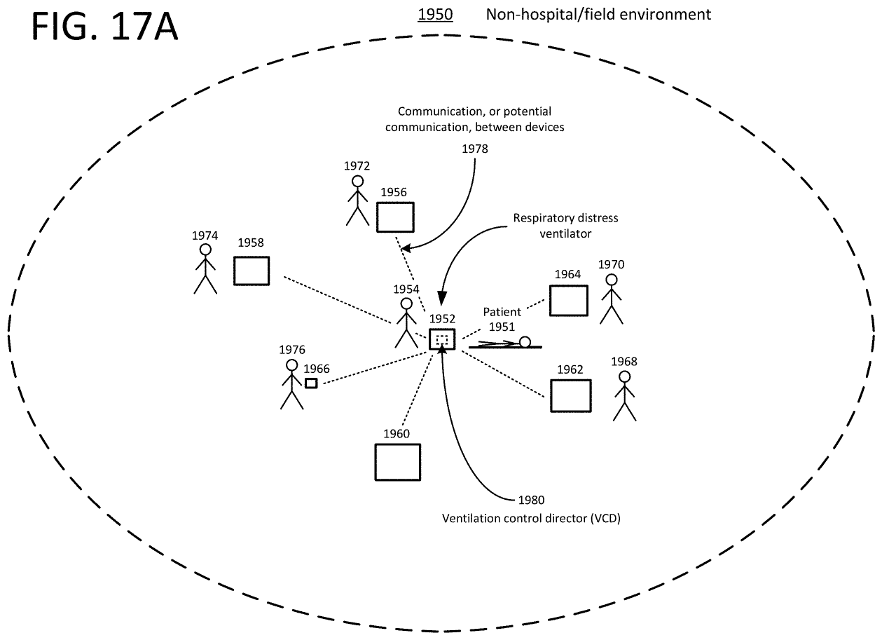

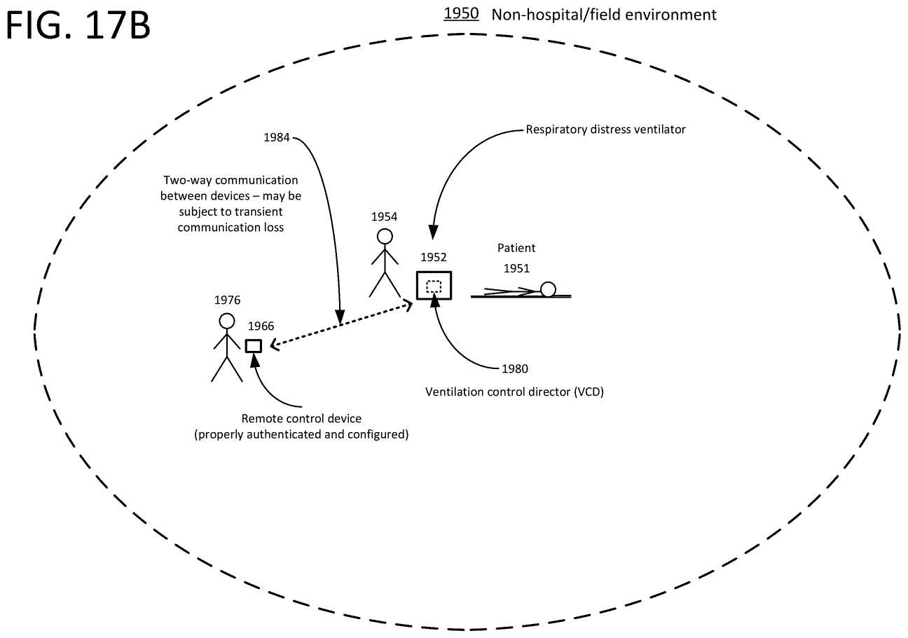

FIG. 17 illustrates an example of an emergency care setting 1900 in accordance with some embodiments, including remote control of the respiratory distress ventilator 1902 via an offsite cloud-based monitor 1912 at an offsite location 1906, via remote control signals 1904 sent from the offsite monitor system 1912 to the VCD 1916 of the respiratory distress ventilator controller 1914, and including one or more forms of CLC 1918. In the implementation depicted, both a local user/care provider 1908 and an offsite user/care provider 1910 are shown. In various embodiments, local remote control of the respiratory distress ventilator 1902 may or may not be included or optionally included.

FIGS. 17A and 17B illustrate examples of a non-hospital emergency care environment 1950, such as a field environment, including a respiratory distress ventilator 1952 and multiple other devices 1956-1964. Often by its nature and by necessity, such an environment may be, relative to a typical hospital environment, in various ways, more spontaneous, less planned, less static, less stable, less pre-configured and less structured. In some embodiments, respiratory distress ventilators, while capable of effective operation in hospitals or similar environments, are also capable of effective operation in various non-hospital emergency care environments. In a non-hospital emergency care environment, many devices and operators may be present, the particulars of which may not be entirely pre-planned. Furthermore, devices (including the respiratory distress ventilator 1952) and operators may arrive and leave at different times, which also may not be entirely pre-planned, and some devices may be subject to frequent movement or physical instability. Some of the devices 1956-1964 may be capable of operationally integrating with the respiratory distress ventilator 1952, which may in some instances include being capable of remote control of mechanical ventilation provided by the respiratory distress ventilator 1952 to the patient 1951. The devices 1956-1964 may include, for example, other medical devices, such as a CCM or defibrillator, which may operationally integrate with the respiratory distress ventilator 1952. For example, such integration may include coupling with the patient 1951 to obtain and provide patient parameter signaling. It may also include providing a display, GUI and/or controls that may be integrated with, or include aspects that relate to, operation of the respiratory distress ventilator 1952 (examples of which are described, for example, with reference to FIGS. 1-7). Furthermore, some of the devices 1956-1964 may be capable of remote control of the respiratory distress ventilator 1952, while some may not. Various communications and signaling, including wireless signaling, within the non-hospital emergency care environment 1950 may include signaling unrelated to operation of the respiratory distress ventilator 1952 or even to patient care. Moreover, communications in the non-hospital emergency care environment 1950, such as over one or more wireless networks, may be uneven and potentially subject to frequent interruption and re-establishment. In some embodiments, the respiratory distress ventilator 1952 is configured for efficient, effective and safe operation even in non-hospital environments with challenges that may include the foregoing. In some embodiments, software relating to such operation may be stored in memory of the controller of the respiratory distress ventilator 1952, including the ventilation control director (VCD), examples of which are described with reference to FIGS. 11-25.

In some embodiments, the VCD 1980 may include software implementing rules or procedures for detecting and identifying other devices, for determining required authorization and required configuration of other devices, and for operationally integrating with other devices. The VCD 1980 may also include, or otherwise have access to, stored information for use in these regards. In some embodiments, the VCD 1980 may detect changes over time in present devices or operators, and the respiratory distress ventilator 1952 may operationally integrate with one or more appropriate devices, which in some cases may include being remotely controlled by a particular device. The VCD 1980 may further include software implementing rules or procedures for selection, for remote control, of a device between multiple properly authorized and properly configured devices, or for switching between different remote control devices, when appropriate. The VCD 1980 may also include software implementing rules or procedures that take into account particular identified communications-related conditions or patterns, such as may include short or long losses of communication. In some embodiments, one or more other devices may themselves also include software and stored information for use in communication and operational integration with the respiratory distress ventilator 1952.

As depicted in FIG. 17A, at the non-hospital emergency care environment 1950, a respiratory distress ventilator operator 1954 operates the respiratory distress ventilator 1952 in providing treatment to a patient 1951, which may include delivering mechanical ventilation to the patient 1951. The emergency care environment 1950 may include multiple potentially disparate devices other than the respiratory distress ventilator 1952, which are represented as other devices 1956-1964. The other devices 1956-1964 may include, for example, portable devices, medical or treatment devices, computers and computerized devices, handheld devices such as tablets and smartphones, and others. Some or all of the other devices 1956-1964 may be operated by one or more operators, represented as operators 1968-1976, while some may operate at some or all times without an operator or automatically. Various of the devices 1952, 1956-1964 and operators 1954, 1968-1976 in the environment 1950 may arrive and leave at various times, so that the devices and operators present in the environment 1950 may vary over time.

In various examples, one or more of the other devices 1956-1964 may be capable of operationally integrating with the respiratory distress ventilator 1952, and potentially also with each other. For example, the other devices 1956-1964 may include a CCM, defibrillator and/or a tablet, which may operationally integrate with the respiratory distress ventilator 1952, various examples of which are described with reference to FIGS. 1-7. However, the other devices 1956-1964 may also include devices that are not capable of operationally integrating with the respiratory distress ventilator 1952. In some examples, devices in operational integration with the respiratory distress ventilator 1952 may be coupled with the patient 1951, and/or may include sensors or other components for collection of signals from the patient 1951 that may be indicative of patient physiological parameters, some examples of which are described with reference to FIGS. 1-7. While not depicted in FIGS. 17A and 17B, devices or sources other than the respiratory distress ventilator 1952 may further include one or more offsite devices or sources that may be capable of operationally integrating with the respiratory distress ventilator 1952, examples of which are described with reference to FIGS. 2, 4 and 6. In some cases, this operational integration may include remote control of the respiratory distress ventilator 1952.

The respiratory distress ventilator 1952 includes the VCD 1980, examples of which are described, for example, in the Summary section and with reference to FIGS. 10-25. In the example depicted in FIGS. 17A and 17B, the VCD 1980 is implemented to include software stored in memory of the respiratory distress ventilator 1952.

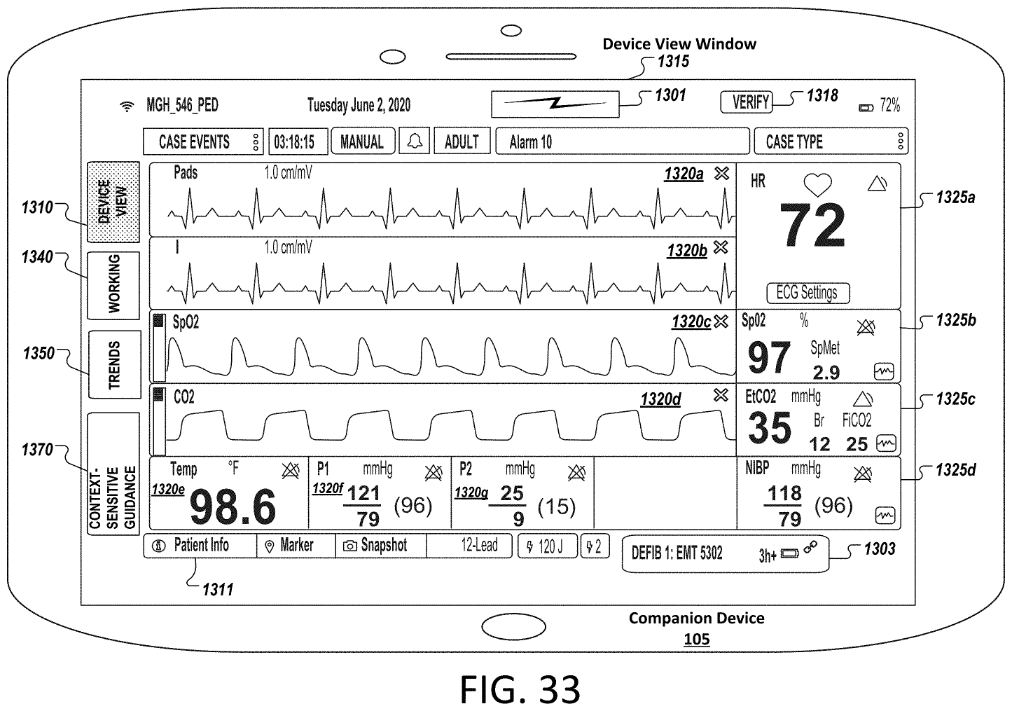

The respiratory distress ventilator 1952, and various of the other devices 1956-1964, may include controls for use by their operators (examples of controls of various devices are described with reference to FIGS. 28A-C and FIGS. 33-35). The respiratory distress ventilator 1952 may be capable of wired and/or wireless two-way communication with some or all of the other devices 1956-1964 via wired or wireless connection, and various of the other devices 1956-1964 may be capable of wired and/or wireless two-way communication with each other.

In some embodiments, one or more of the other devices 1956-1964 may be capable of remote control of mechanical ventilation provided by the respiratory distress ventilator 1952 to the patient 1951, such as may include control via input (such as may relate to settings-related commands), such as selections or entries via device controls, by the respective operator of the device, or in some cases or at some times, by the device automatically.

In some embodiments, in an instance in which remote control is established with a particular remote control device, the VCD 1980 may manage and implement control of mechanical ventilation by the respiratory distress ventilator 1952 including use of fault mode condition analysis, examples of which are described with reference to FIGS. 12-25, which may affect whether the particular remote control device or the VCD 1980 itself controls at a particular time.

Furthermore, the VCD 1980 may only allow remote control by a properly authorized and properly configured device or source. In particular, in some embodiments, the VCD 1980 may, via two-way communication with the device, determine whether the device meets one or more specified authorization requirements and one or more specified configuration requirements (such as device-related, hardware and/or software configuration requirements). In various embodiments, for example, the respiratory distress ventilator 1952, the device, or both, may be configured to detect when the other is present, such as within a particular distance, and/or if other conditions are satisfied, e.g., the other is powered on. Communication between them may be initiated by the respiratory distress ventilator 1952 or the device (e.g., the device may send an offer or request to remote control the respiratory distress ventilator 1952, or the respiratory distress ventilator 1952 may send an offer or request to be remote controlled by the device). The device may send (or, e.g., the VCD 1980 may request, and then the device may send) information to the respiratory distress ventilator 1952 to allow the VCD 1980 to determine that the device (or the device and the operator) satisfies the authorization and configuration requirements (e.g., the device may send device and/or operator identification information, authorization password or code information, device configuration information, and/or other information). The VCD 1980 may only allow remote control by the device if the VCD 1980 determines that the device meets these requirements.

Additionally, in environments including multiple potential remote control devices, the VCD 1980 may include software implementing rules or procedures for managing remote control in this context. This may include, for example, rules or procedures for making authorization and configuration determinations for each of several devices, or for different devices at different times. It may further include rules or procedures for making a selection, for remote control, between multiple properly authorized and properly configured devices, or rules or procedures for switching from one remote control device to another under specified conditions, for example.

FIG. 17B shows some of the components of the non-hospital emergency care environment 1950 as depicted in FIG. 17A, including one of the other devices, specifically, the device 1966 (other devices not shown). The device 1966 may be, e.g., a CCM, defibrillator, or tablet in two-way communication 1984 with the respiratory distress ventilator 1952. In the depicted example, the VCD 1980 has determined that the device 1966 is properly authorized and properly configured, and remote control by the device 1966 has been established.

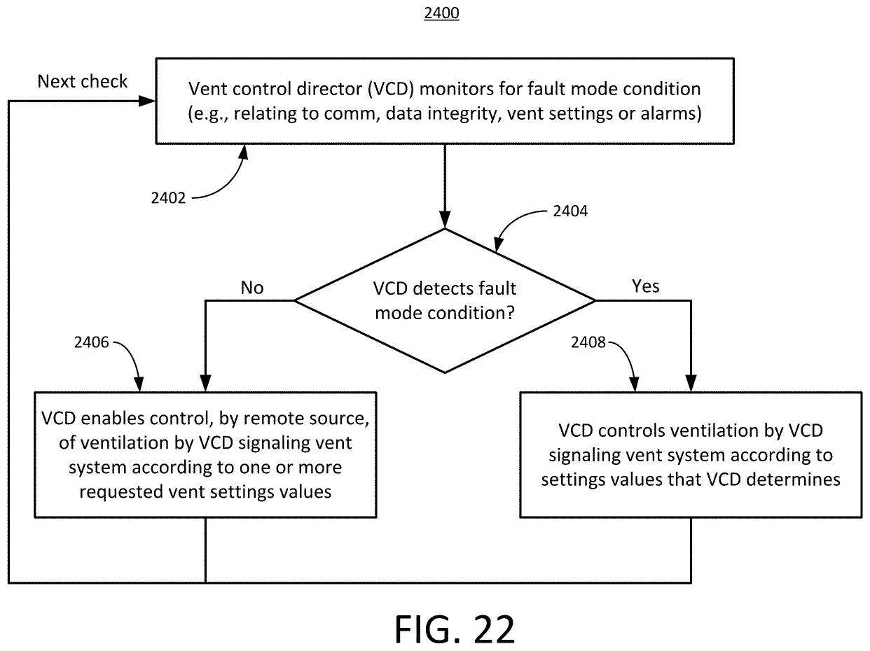

As described with reference to FIG. 16, for example, in some circumstances, communication between the respiratory distress ventilator 1952 and the remote control device 1966 may be cut off, or otherwise lost, at a particular time. This may be an example of a fault mode condition that causes the VCD 1956 to control mechanical ventilation by the respiratory distress ventilator 1952, such as based on one or more back-up, default, currently in use, most recent or last received valid settings values, for example, as described with reference to FIGS. 22-25.

Additionally, in some embodiments, the VCD 1980 may include software implementing rules or procedures specifically for use in, for example, transient loss of communication situations, such as situations in which communication is lost or cut off for a very short period of time (e.g., a specified fraction of a second or several seconds). For example, in some embodiments, the VCD 1980 may be configured to use last received valid settings for loss of communication up to a specified first threshold period of time (herein referred to as a transient loss of communication), but switch to use of different back-up settings if the loss of communication extends longer than the first threshold period of time (herein called a non-transient loss of communication). In various embodiments, various different rules may be used in transient loss of communication situations, relative to non-transient loss of communication situations, such as may relate to use of different back-up, last-received valid, or other settings values in such situations, for example. Still further, in some embodiments, the VCD 1980 may be configured to store information on recent communication, may use one or more algorithms to identify communication related patterns in recent communications, and may use rules for determining settings values, which rules are based at least in part on a detected recent communication related pattern. As one of many possible examples, an identified recent communication related pattern of frequent transient losses of communication may indicate an increased probability of communication being re-established relatively quickly during an immediately subsequent future period of time, and may trigger some particular measure to be taken during the immediately subsequent future period of time, such as a slight increase in the first threshold time period, or use of slightly different particular back-up settings.

It is to be noted, however, that, since no communication between devices is truly instantaneous (given various factors such as, e.g., normal latency, normal network traffic, etc.), in some embodiments, very short, and normal, delays in communication (e.g., in various embodiments, up to a threshold of a specified fraction of a second or several seconds) may not even be considered or defined as transient losses in communication. Furthermore, in some embodiments, a transient loss of communication may be considered or defined as a loss of communication of above a specified maximum normal delay threshold period of time (considered or defined to be associated with normal delays in communication) and up to a specified maximum threshold period of time associated with transient delays, for example. In some embodiments, normal delays do not result in any settings values related change being implemented by the VCD 1980, which may result in use of last received valid settings during normal delay periods, for example.

In some embodiments, the respiratory distress ventilator 1952 continuously sends, such as via wireless communication, data to the remote control device 1966, including current settings values in use. During remote control, the remote control device 1966 may periodically send settings values commands to the respiratory distress ventilator 1952, such as to change particular settings values to particular new settings values. The settings values commands may reflect settings values selected or entered by the operator 1976 of the remote control device 1966 via controls of the remote control device 1966. Additionally, however, the respiratory distress ventilator 1952 may have controls through which the operator 1954 of the respiratory distress ventilator 1952 may also make selections or entries resulting in settings values commands, potentially even during control by the remote control device 1966. In some embodiments, this can sometimes lead to a conflict at a given time between a command received by the respiratory distress ventilator 1952 via a communication from the remote control device 1966 and an unmatching command (e.g., a command that specifies a different value for a particular setting) received by the respiratory distress ventilator 1952 and based on one or more selections or entries by the operator 1954 of the respiratory distress ventilator 1952. In some embodiments, the VCD 1980 includes software implementing rules or procedures for handling such conflicting situations. For example, in some embodiments, in such a conflicting situation, a command received by the respiratory distress ventilator 1952 based on a selection or entry by the operator 1954 of the respiratory distress ventilator 1952 is given precedence or priority, and is implemented instead of the conflicting and unmatching command received by the respiratory distress ventilator 1952 via the communication from the remote control device 1966. However, in various embodiments, various other conflict resolution and management rules or procedures may be followed, which may include, for example, informing either or both operators 1976, 1954 of the conflict and prompting one or both for confirmation, acceptance of the conflicting command, command change, or to take other steps.

Furthermore, communication losses between the respiratory distress ventilator 1952 and the remote control device 1966 can lead to situations in which, for example, the supposedly current settings values (or other information, such as patient parameter information) available at the remote control device 1966, and potentially viewed by the operator 1976 on the controls of the remote control device 1966, are actually not current and are therefore incorrect. For example, they may reflect current settings values as of seconds or minutes ago, before communication was cut off, and the settings values may have changed during the cut off period. As a result, the operator 1976 may potentially make selections or entries, resulting in associated commands, based on incorrect information, so that the selected or entered settings values by the operator 1976, and the resulting commands, may be suboptimal or ill-advised, being based on incorrect relevant information. Furthermore, in cut off communication situations, the operator 1954 of the respiratory distress ventilator 1952 may be in receipt of non-current information sent by the remote control device 1966. In some embodiments, the VCD 1980 (and possibly other devices, as well) includes aspects and software for implementing rules or procedures to address or mitigate problems associated with such situations. For example, in some embodiments, the VCD 1980 may cause an alarm or alert (e.g., a light, colored light, other visual display or sound) to be provided to the operator 1954 of the respiratory distress ventilator 1952, such as may warn or alert the operator 1954 that one or more commands received from the remote control device 1966 may reflect consideration of particular basis information that is not current (e.g., at the time that the one or more associated selections or entries were made by the operator 1976 of the device 1966, the operator 1976 was in receipt of current settings values information that was actually not current and therefore incorrect or potentially incorrect). The alarm or alert may further indicate a degree by which the particular basis information is non-current (such as a range of seconds or minutes), and/or the alarm or alert may be different, such as of greater magnitude (e.g., a louder tone, a brighter light, a different color light) if the degree is greater (e.g., if the amount of time that the particular information is non-current is greater). In some embodiments, the remote control device 1966 may use time of receipt information or a time stamp of last information from the respiratory distress ventilator 1952, and alerts or alarms may be provided to the operator 1976 accordingly. In some embodiments, normal delays in communication are not considered to make received information non-current, however.

FIGS. 18-21 illustrate examples of fault mode conditions of various types, e.g., relating to communication 1102, data integrity 1104, ventilation settings 1106 and patient physiology 1108 as depicted in FIG. 12. As described with reference to, e.g., FIGS. 12-17, in some embodiments, a VCD of a respiratory distress ventilator may manage aspects relating to fault mode conditions, and may also manage and implement aspects relating to control of a respiratory distress ventilator, such as by internal control, by enabling or allowing remote control, or with aspects of both.

FIG. 18 illustrates a table 2000 of examples of communication related fault mode conditions 1102, in accordance with some embodiments. Communication related fault mode conditions 1102 may include, e.g., conditions in which the VCD detects that communication with a remote control source has been lost 2002 (or, in some embodiments, is insufficient or too slow) or conditions in which the VCD detects attempted control by an unauthorized or improperly configured source 2004.

In various embodiments, various error detection and/or correction (EDC) schemes may be utilized to reduce bit-error rates (BER) and subsequent communication faults, as well as detection or errors. In some embodiments, for example, an acknowledgement (ACK) and/or negative acknowledgement (NACK) software technique or scheme may be utilized. The ACK/NACK scheme may be used, for example, in establishing whether communication is currently present, which may be required for remote control to be permitted. Simple cyclic redundancy checks (CRC) may be performed. Alternatively, block or convolutional codes may be employed. Alternatively, more complex EDC methods may be utilized. In some examples, the EDC methods may be specifically tailored to the unique set of requirements presented by the activity of remote control.

In various embodiments, in remote control applications, the controlled device communicates with the remote controller. The communication may be wired or wireless, synchronous or packet-based. Typically, the controlled devices have a limited set of commands/controls/actions/moves that they can execute. For example, the remote control often occurs in noisy environments in ambulances, helicopters or other aircraft, as well as electromagnetic interference environments. Actions by the controlled devices, combined with environmental disturbances, may cause a change in the parameters describing the state of the system. Controlled devices may need to communicate with the remote controller regarding their current state and get further instructions. The information flow will be subject to noise; therefore, system performance may depend upon encoding the transmissions against channel noise. Furthermore, there may be added constraints with remote control due to: the need for a real-time response to transmissions; causal encoding and decoding (bits of the code can depend only on past events; and, the tradeoff between the accuracy and reliability of the information and the amount of delay allowed for the real-time communication. In some embodiments, coding schemes optimized for remote control applications may be employed, such as, for instance, trajectory codes or tree codes, as described in “Error Correcting Codes for Automatic Control”, IEEE Transactions on Information Theory, Vol 55, No. 7, July 2009.

In some embodiments, the VCD is configured to only allow remote control by an authorized or properly configured source. For example, in some embodiments, it is necessary ensure that unauthorized or improperly configured devices or systems are not permitted control the respiratory distress ventilator, such as by controlling aspects of mechanical ventilation of the respiratory distress ventilator. As such, in some embodiments, only certain devices or systems, or devices or systems that meet predetermined authorization or configuration requirements, are allowed by the VCD to remotely control the respiratory distress ventilator (e.g., subject to fault mode condition analysis).

For example, communication signaling 1102, as depicted in FIG. 12, may include a set of data sent by a remote device to be received (whether directly or via one or more intermediary entities) by the respiratory distress ventilator. The set of data may include information that can be used by the VCD in determining whether to permit the device or system to remotely control the respiratory distress ventilator (e.g., subject to fault mode analysis). In some embodiments, for example, the VCD may compare the received set of data to stored data (such as may be stored in the memory of the controller of the respiratory distress ventilator), where both the received set of data and the stored data may relate to authorization or configuration. Based at least in part on the comparison, the VCD may determine whether the remote device or system meets the authorization and/or configuration requirements, and may permit or not permit remote control accordingly. In some embodiments, only certain devices or systems, or certain types or groups of devices or systems, may meet these requirements. For example, the received set of data may specify certain authorization or configuration information, which may be compared to, and must match or otherwise comport with stored data specifying required authorization or configuration information, in order for the VCD to permit the remote control.

In some embodiments, the remote device or system may send the set of data to the respiratory distress ventilator based on a user-initiated request (such as via a display/GUI) made at the remote device or system, the respiratory distress ventilator, or another device or system. However, in some embodiments, the data may be sent without user initiation. For example, the remote device or system may be configured to be automatically triggered to send the data, or the VCD may be triggered to request it, by any of various triggering conditions. For example, where the remote device or system is a local device, the data sending may be triggered merely by the respiratory distress ventilator and the remote device coming sufficiently close to each other, as may be detected by either or both of the devices. As another example, in the case of a modular environment such as depicted in FIG. 8, data sending may be triggered by the respiratory distress ventilator and the remote device being joined, attached or docked together or at the same dock or docking station. In some embodiments, even if the VCD determines that remote control is permitted, a user may be prompted to, and may be required to, confirm that remote control should occur. The prompting and confirmation may occur at the respiratory distress ventilator, the remote device or system, or another device or system.

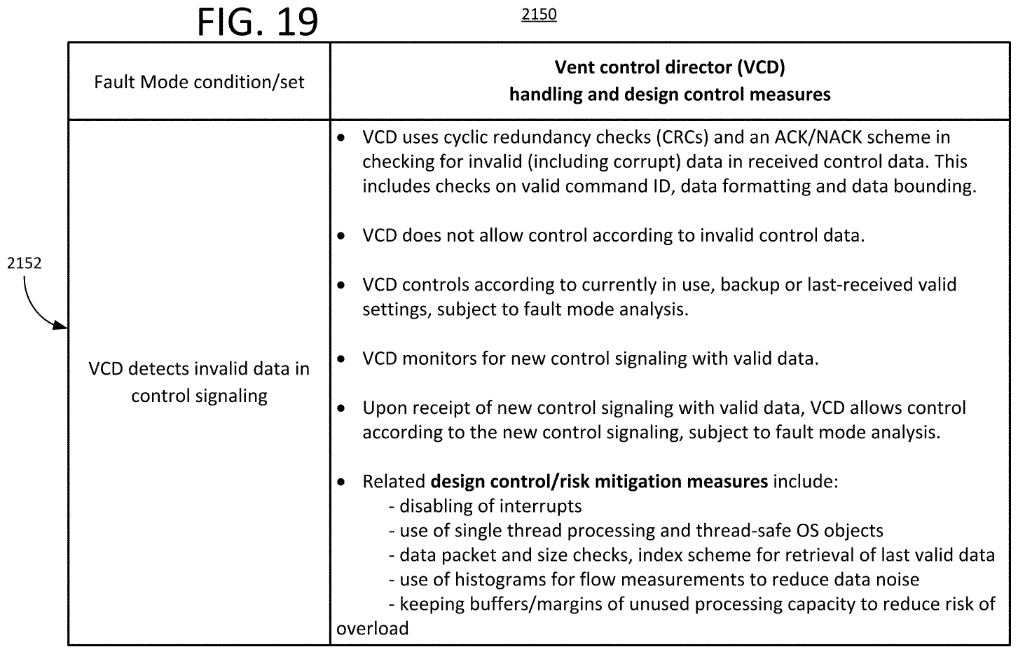

FIG. 19 illustrates a table 2150 of examples 2152 of data integrity related fault mode conditions, in accordance with some embodiments. As depicted in FIG. 19, data integrity related fault mode conditions may include, e.g., conditions in which the VCD detects invalid request/command data (e.g., not usable, corrupt or otherwise not implementable, for any of a variety of potential reasons).

In some embodiments, for example, an ACK/NACK scheme is used in checking for invalid received control data, as well as in confirming communication. Furthermore, in some embodiments, cyclic redundancy checks (CRCs), or one or more other error detection methods or techniques, may also be utilized.

For example, in some embodiments, a control request/command, received by a VCD of a respiratory distress ventilator from a remote control device or system, is checked for satisfaction of particular requirements, and receipt of the control command may also be used to confirm that communication exists. Requirements for validity may relate, for example, to a received data packet, proper/allowed data packet size, formatting, framing, presence of start and stop delimiters/bounding, valid length, and confirmation of one or more aspects of the control command not being corrupt. Additionally, a CRC may be performed.

In some embodiments, a predetermined set of commands may be stored in the memory of the controller, and are accessible by the VCD. As a requirement of validity, the VCD may check to ensure that a received command matches at least one of the predetermined set of valid commands. For example, in some implementations, each command may have associated command identification data (command ID), such as an associated number or alphanumeric string. A received command ID may be required to match one of the set of valid commands, among other requirements.

Furthermore, in some embodiments, as a requirement, requirement of validity, or an additional requirement of validity, the VCD may require receipt, such as periodically or at regular intervals, such as once every 10 seconds (or, e.g., once every fraction of a second, once every 1-10 seconds, or once every 10-30 seconds), of particular enablement identification data (an “enable ID”) from the remote control device or system. A set of predetermined enable IDs may be stored, such in the memory of the controller, where each of the enable IDs relates to a particular set of commands, each of which particular sets of commands may be, for example, a subset of all possible commands. For example, each particular set of commands may relate to one or more particular types, categories or breadths of commands, such as, for example, test function related commands, monitoring function related commands and ventilation control related commands. A particular enable ID may relate to one or more of the particular sets of commands, such as, for example, test function related commands only, monitoring function related commands only or ventilation control related commands only, or may relate to two or more sets of commands, such as, for example, to monitoring related commands and ventilation control related commands, or to monitoring related commands, ventilation control related commands and test related commands. In various embodiments, in order to allow remote control relating to the particular one or more sets of commands associated with a particular enable ID, or in order to allow communication, or command related communication, relating to the particular one or more sets of commands with the remote control device or system, or in order to allow communication, or command related communication, of any type with the remote control device or system, the VCD may require that an enable ID be received from the remote control device or system that matches the particular enable ID at every particular time period or interval, for example.

In some implementations, if the packet meets the requirements, it is deemed valid, the VCD may respond by sending an ACK data packet to the device, and remote control is allowed. However, if the packet does not meet the requirements, it is deemed invalid, the VCD may not respond by sending an ACK data packet to the device (or may respond with a different ACK data packet than if the control command meets the requirements), and remote control is not allowed.

In some embodiments, a particular protocol is used in connection with the ACK/NACK scheme. For example, in some implementations, the VCD may await an ACK or NACK reply from the device for a predetermined reply period of time, e.g., 500 ms. If no reply is received in the predetermined reply period (e.g., a timeout occurs), then the VCD may or may not immediately send additional communications and may again await a response (e.g., a maximum of between 1-20 such attempts may be made, such as 2-5 or 3). In some embodiments, the predetermined reply period, the number of tries, and/or the number of timeouts may be determined or optimized based in part on, or correlated with, a possible or anticipated bit error rate, or maximum bit error rate, associated with the reply or communication link.

Furthermore, in some embodiments, an ACK/NACK and retry scheme may be utilized, or additionally serve the purposes of, e.g., effectively regulating or limiting data packet rate (or other data receipt rate) at which the VCD and respiratory distress ventilator receive data from the device or system. For example, the scheme may be used in limiting the data packet receipt rate to a flow that is manageable by the respiratory distress ventilator, such as without causing problems, loss of data, overflow, delay or an unacceptable amount of delay, or reducing optimization of overall operation. For example, in some embodiments, if the rate of data packet receipt is unacceptably high, the VCD may control or determine the timing or rate of response(s) in order to throttle, control or limit the rate to a maximum acceptable level.

In some embodiments, the VCD does not allow remote control according to request/command data that is determined to be invalid. In the event that invalid request/command data is received, the VCD may monitor for and/or await new, valid request/command data. When valid, new data is received, the VCS may allow control accordingly, subject to fault mode condition analysis.

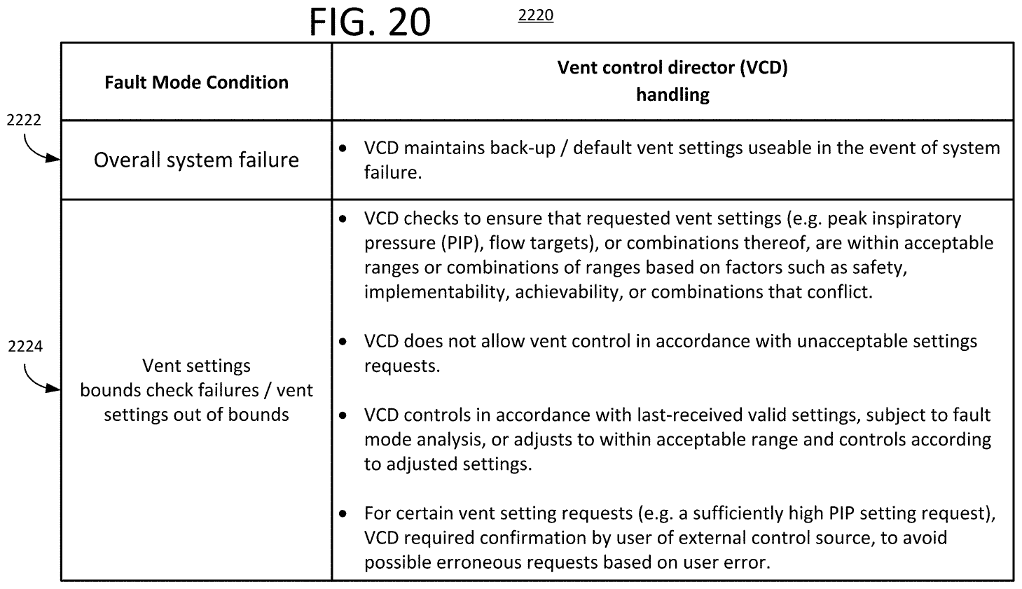

In some embodiments, the VCD may be configured to determine whether control according to newly received, valid request/command data may represent too large or too abrupt a change in one or more ventilation settings, which may represent a ventilation settings related fault mode condition, further examples of which are provided with reference to FIG. 20. For example, this could be the case if the mechanical ventilation apparatus of the respiratory distress ventilator would be unable to immediately make such a large or abrupt change (or combination of changes), or if such a large or abrupt change (or combination of changes) might cause risk to the ventilation system or to the patient, or an unacceptable or unwarranted degree of risk. In various embodiments or instances, in such cases, the VCD may respond in various ways. For example, the VCD may algorithmically specify a gradual increase or decrease of over time of one or more settings values, such as may include adjustments of the one or more settings values over time. The rate or rates of increase or decrease may be limited to a possible, optimal or safe rate, for example.

In various embodiments, until new, valid request/command data is received (or, in instances in which communication is lost or insufficient, until it is re-established), the VCD may internally control mechanical ventilation by the respiratory distress ventilator in various possible ways. In some embodiments, the VCD may control the mechanical ventilation according to currently in use, most recent or last received valid, backup or default settings, until new, valid request/command data is received (subject to fault mode condition analysis). Furthermore, in some embodiments, the controller of the respiratory distress ventilator and the VCD may be configured to use an indexing scheme to allow fast, efficient, or optimized retrieval of last received valid, backup or default settings, which may allow faster or more efficient control.

In some embodiments, aspects of the respiratory distress ventilator controller or VCD include optimizing features, such as may mitigate risk and/or increase optimization in connection with control of mechanical ventilation provided by the respiratory distress ventilator. For example, such optimization may take into account the priorities of VCD operation, which may include some degree of emphasis on, or prioritization of, e.g., avoiding stoppage, freeze, hold-ups or delay, sometimes over, e.g., efficiency or conservation of resource usage with respect to processing, for example. Additionally, such optimization may take into account the importance of minimizing stoppages, delays or incompatible determinations, which, in some instances, can pause, delay or reduce the degree of speed of implementation of optimal ventilation settings adjustments. This, in turn, can reduce the optimization of the provided ventilation, potentially reducing optimal patient care, or creating or increasing patient risk. Optimizing features may include, for example, as determined to be appropriate, disabling of interrupts, e.g., in order to prevent temporary stoppages of VCD operation, and use of single thread processing aspects, which can reduce temporary processing hold-ups and stoppages in connection with VCD operation.

In some embodiments, single thread processing and/or multithread processing may be utilized. In a single thread process, instructions are executed by a processor in a single sequence. By contrast, in multithread processing, multiple parts of a program are executed concurrently, in multiple concurrently executed threads (which are sequences of programmed instructions). Multithread processing can be faster and more efficient than single thread processing, allowing shared access to resources by multiple threads. However, multithread processing tends to be much more complex and can be more prone to lead to errors or hold-ups. This can be due to the much greater complexity of the multithread programming, or can be related to required locking of a resource in multithreading, which limits access to a particular resource in order to prevent inconsistencies, for example. In some embodiments or areas, single thread processing is utilized, since it can be simpler and less prone to potentially lead to errors or hold-ups, for example, since no locking of resources is required. In some embodiments, however, multithread processing is utilized or is also utilized, given its advantages, but various optimizing features may be employed in connection with its use, as described below, to minimize or reduce disadvantages.

In multithreaded aspects, optimizing features may include use of threadsafe aspects, for example, so that multiple threads may utilize common resources with reduced delay or unpredictability. Such optimizing features may include, for example, use of: stateless implementation aspects (e.g., use of stateless deterministic functions); immutable implementation aspects (e.g., a class instance which has an internal state that cannot be modified after it has been constructed); threadlocal fields (e.g., classes that don't share state between threads); synchronized collections (e.g., using a set of synchronization wrappers included within a collections framework), concurrent collections (e.g., which divide their data into segments); atomic objects (e.g., which allow performance of operations without using synchronization); synchronized methods (e.g., where only one thread can access a synchronized method at a time, while blocking access to this method from other threads), synchronized statements (e.g., synchronizing an entire method), using other objects as locks, such as reentrant locks or read/write locks (e.g., using an external entity to enforce exclusive access to a resource); caveats (e.g., avoiding using strings for locking purposes); and, volatile fields (e.g., storing and reading, a countervariable in/from a main or other memory, and not in a cache).

Optimizing features may further include, as appropriate, use of histograms or histogram filters for flow measurements by the respiratory distress ventilator, such as in filtering for and reduction of data noise. This may include, for example, using histogram filters, or one or more other types digital filters, in determining most probable signal values, and using the determined most probable signal values in filtering out data noise. Optimizing features may further include, for example, maintaining a predetermined or optimized buffer or unused portion of processing capacity or memory, in order to reduce risk of overload, freeze or stoppage.

FIG. 20 illustrates a table 2220 of example ventilator settings related fault mode conditions, in accordance with some embodiments. Ventilator settings related fault mode conditions may include, for example, overall system failure 2222, which may include situations in which control by the VCD according to remote control fails. In such instances, the VCD may be configured to control the mechanical ventilation apparatus according to, e.g., backup, default, currently in use, most recent or last received valid settings values. Furthermore, the controller and VCD may be configured to implement such control even when experiencing any of various failures, e.g., failure or inability to receive, interpret, process or implement remote control data or commands.

Ventilator settings related fault mode conditions may also include fault mode conditions relating to ventilation settings check failures 2224, such as if one or more requested vent settings, or a combination thereof, are not possible or are out of allowed bounds. These can include, e.g., VCD checks to ensure that requested vent settings (e.g. PIP, flow targets), or combinations thereof, are within acceptable ranges or combinations of ranges based on factors such as safety, achievability or capability of being implemented. For example, in some embodiments, permitted ranges may be preconfigured and based on the design and limits of the system, such as maximum or minimum settings that are achievable, controllable or stably or non-erratically controllable, or may be based on preconfigured rules but dependent on particular operating conditions or other settings. In various embodiments, in particular situations, the controller and VCD may handle out of bounds settings commands in various ways. In some embodiments, for example, a warning or other message may be triggered and provided to a user regarding a requested setting. In various instances, the user may be warned and prompted, such as via CSG, to confirm the change, or given a message the requested change is not permitted. The message might include additional detail, such as the reasons why the requested change is not permitted, or a maximum smaller change that may be permitted, for example. In some embodiments or instances, the VCD may not control in accordance with a setting (or settings) request/command that is determined to be out of allowed/permitted bounds, and may instead control according to, e.g., back up, default, currently in use, most recent or last received valid settings.

In other embodiments or instances, the VCD may be configured to determine an adjusted setting(s) based at least in part on the setting(s) associated with the setting request/command. For example, in some instances, if a requested setting is above a permitted range, the VCD may determine an adjusted setting that is at the maximum of the permitted range, and if a requested setting is below a permitted range, the VCD may determine an adjusted setting that is at the minimum of the permitted range. The VCD may then control the mechanical ventilation apparatus of the respiratory distress ventilator according to the determined adjusted setting. Furthermore, in some instances, combinations of two or more settings may be constrained within ranges that are interdependent, such that the permitted range of each is at least in part dependent on the permitted range or one or more others. For example, such interdependent settings can include breath rate, I:E ratio and inspiratory time, among others. In such cases, in various ways, the VCD may determine adjusted settings for one or more of the requested settings such that the combination of settings is permitted, for example, and may control accordingly.

Furthermore, in some implementations, for some requested settings, or requested settings that fall into particular ranges, the VCD may be configured to obtain user confirmation, authorization or permission of a setting before controlling according to the requested setting. For example, user confirmation may be required regarding a setting change that may impact or create patient risk in some aspects, or may suggest or be related to potential data error. For example, such error could be human/user error, such as a request/command associated with a setting change by a user where the user may have accidentally input an erroneous value, or equipment, hardware or software based error (e.g., caused by an erroneous sensor reading such as may be caused by a misplaced or dislodged probe). For example, in some implementations, a requested PIP or PEEP setting that falls into a range that is at or beyond a particular high threshold, or potentially excessively high, may require user confirmation. Furthermore, in some implementations, when user confirmation is required, the user may be prompted accordingly, such as via a display/GUI or CSG of the remote control device, and/or the respiratory distress ventilator, for example.

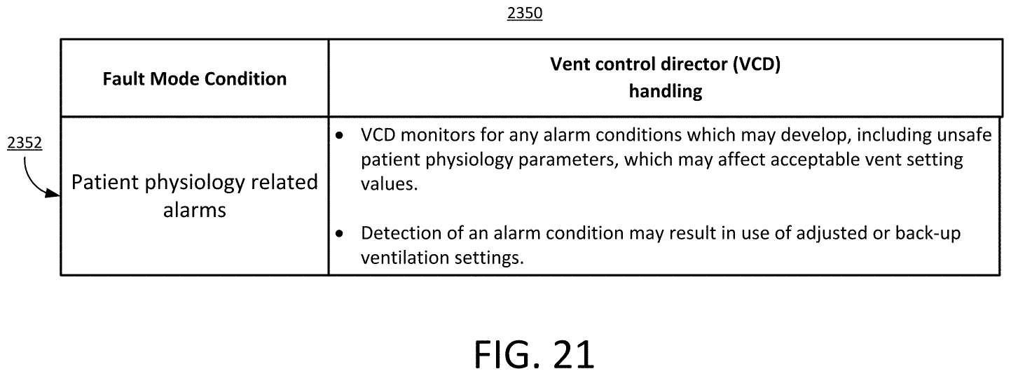

FIG. 21 illustrates a table 2350 of example alarm related fault mode conditions 2352, in accordance with some embodiments. Alarm related fault mode conditions may include, e.g., patient physiology related alarms. In some embodiments, for example, the VCD may be configured to monitor for predefined patient physiological parameter values, such as high risk or unsafe parameter values, which may develop and may render one or more current ventilation setting values impermissible. For example, in some instances, a high risk or unsafe patient physiological parameter value may develop that may render one or more particular current ventilation settings unsafe. In various embodiments, in such instances, that VCD may, for example, control according to adjusted, backup or last received valid settings, including changing current settings as needed. Furthermore, in some implementations, the VCD is configured such that alarms, or some alarms, cause presentation of an alarm presented to a user, such as an visual or audio (e.g., sound or words) alarm, which may, for example, be presented on a display/GUI or CSG of the remote control device and/or the respiratory distress ventilator. It is to be noted that, in some embodiments, the VCD may monitor for, and take action relating to, alarm conditions separately or independently from, or in addition to, other fault mode conditions and fault mode condition analyses, such as by continuously or frequently monitoring for alarm conditions and taking appropriate action accordingly.

FIG. 22 illustrates a method 2400 relating to fault mode condition analysis implemented by a VCD, in accordance with some embodiments, including control by the VCD signaling the ventilation system either according to received ventilation settings values or by settings values that the VCD determines. At step 2402, the VCD monitors for occurrence or presence of a fault mode condition (e.g., relating to communication, data integrity, ventilation settings or alarms). At step 2404, the VCD queries whether a fault mode condition (or more than one) is detected, such as at a predetermined or particular time, for example.

At step 2404, if the VCD does not detect a fault mode condition, then, at step 2406, the VCD enables control, by a remote control source, of ventilation by signaling the ventilation system of the respiratory distress ventilator according to one or more ventilation settings values requested by the remote control source. The VCD then waits a predetermined time period (e.g., 1 second, a fraction one second, or several seconds (e.g., between 0.1 and 1 second, or between 1-10 seconds) and then returns to step 2402.

However, at step 2404, if the VCD detects a fault mode condition, then, at step 2408, the VCD controls the ventilation system of the respiratory distress ventilator according to one or more settings values that the VCD determines (e.g., based on adjusting requested settings, or based on back up, default, currently in use, most recent or last received valid settings). The VCD then waits a predetermined time period (e.g., 1 second, a fraction one second, or several seconds) and then returns to step 2502.

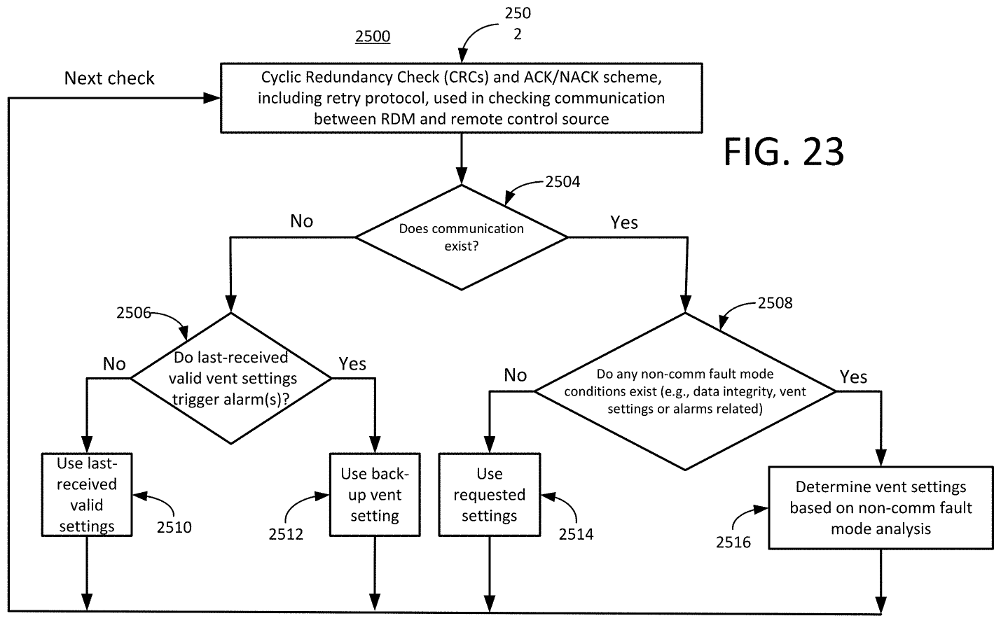

FIG. 23 illustrates a method 2500 relating to fault mode condition analysis implemented by a VCD, in accordance with some embodiments, including details relating to communication related fault mode condition analysis. At step 2502, the VCD performs one or more CRCs and an ACK/NACK scheme, including a retry protocol, in checking communication between the respiratory distress ventilator and a remote control source. At step 2504, the VCD queries whether communication exists (or, in some embodiments, whether sufficient communication, or communication at a sufficient rate, exists), such as at a current time, for example.

At step 2504, if communication is determined not to exist, then, at step 2506, the VCD queries whether last received valid ventilation settings from the remote control source trigger any alarms. In some embodiments, this may provide an additional or backup check to monitoring for settings that trigger any alarms, If not, then, at step 2510, the VCD controls the mechanical ventilation system of the respiratory distress ventilator according to the last received valid settings. If so, then, at step 2512, the VCD controls according to back-up settings.

However, at step 2504, if communication is determined to exist, then, at step 2508, the VCD queries whether any non-communication fault mode conditions exist (e.g., relating to data integrity, ventilation settings or alarms, examples of which are described with reference to FIGS. 19-21). If not, then, at step 2514, the VCD controls the mechanical ventilation system of the respiratory distress ventilator according to the requested settings. If so, then, at step 2516, the VCD controls according to ventilation settings in accordance with non-communication related fault mode condition analysis.

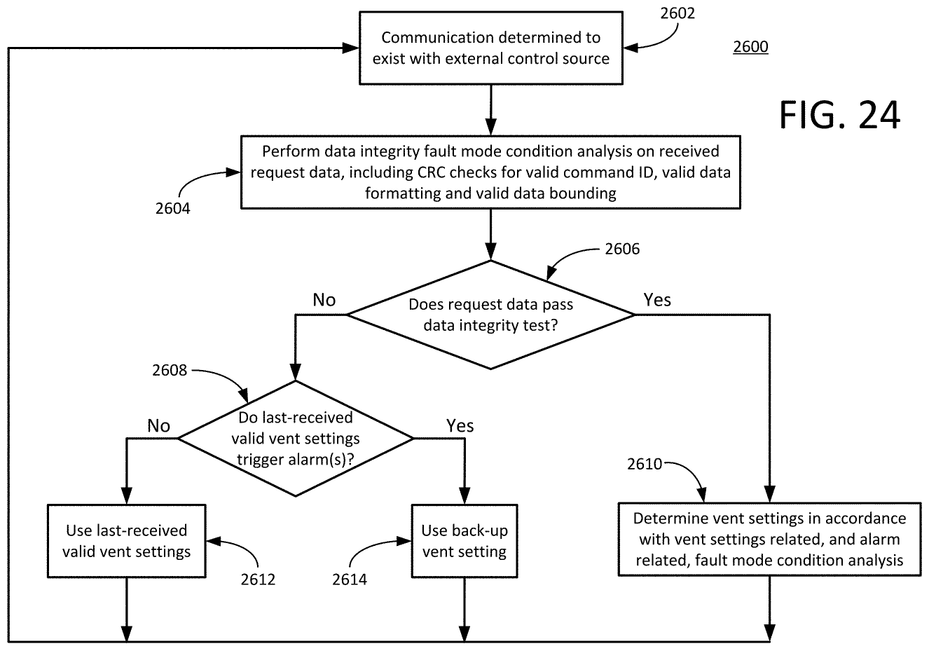

FIG. 24 illustrates a method 2600 relating to fault mode condition analysis implemented by a VCD, in accordance with some embodiments, including details relating to data integrity related fault mode condition analysis. At step 2602, the VCD determines that communication exists between the VCD and a remote control source. At step 2604, the VCD performs data integrity related fault mode condition analysis on received setting(s) request data, including one or more CRC checks for valid command ID, data formatting and data bounding, potentially among other things. At step 2606, the VCD queries whether the setting(s) request data passes the data integrity related fault mode condition analysis (e.g., no data integrity related fault mode condition is determined to exist, such as at a current time).

At step 2606, if the data does not pass the analysis (e.g., at least one data integrity related fault mode condition is determined to exist), then, at step 2506, the VCD queries whether last received valid ventilation settings from the remote control source trigger any alarms. If not, then, at step 2612, the VCD controls the mechanical ventilation system of the respiratory distress ventilator according to the last received valid settings. If so, then, at step 2614, the VCD controls according to backup settings.

However, at step 2606, if the data passes the analysis (e.g., no data integrity related fault mode condition is determined to exist), then, at step 2610, the VCD controls the mechanical ventilation system of the respiratory distress ventilator in accordance with ventilation settings based on ventilation settings related fault mode condition analysis and alarm related fault mode condition analysis.

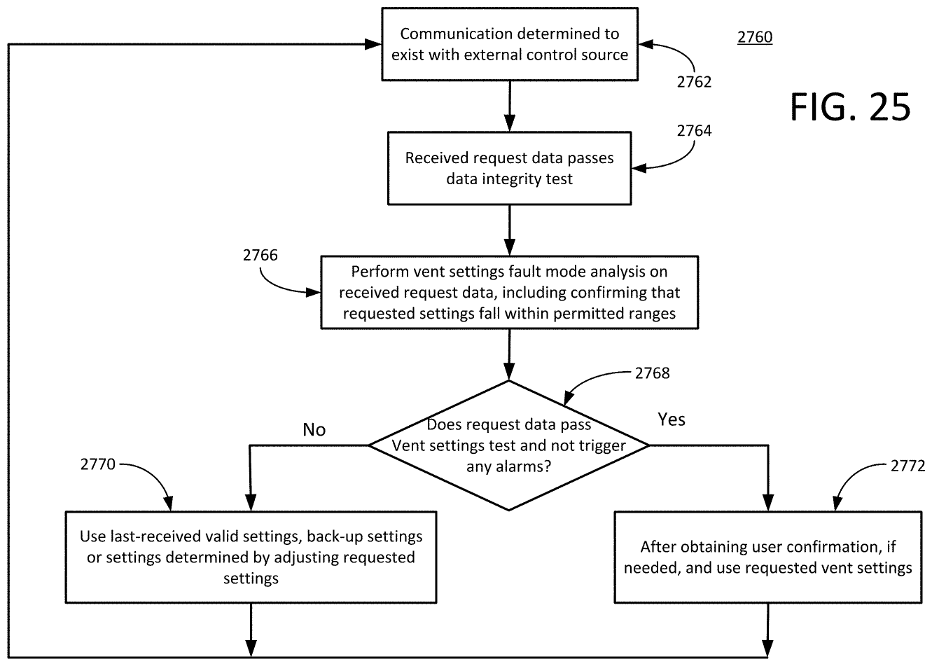

FIG. 25 illustrates a method 2760 relating to fault mode condition analysis implemented by a VCD, in accordance with some embodiments, including details relating to ventilation settings related fault mode condition analysis. At step 2762, the VCD determines that communication exists between the VCD and a remote control source. At step 2764, the VCD determines that the received setting(s) request data passes a data integrity related fault mode condition analysis test (e.g., no data integrity related fault mode condition is detected). At step 2766, the VCD performs ventilation parameter settings related fault mode condition analysis on received setting(s) request data, including confirming that the requested ventilation parameter setting(s) value(s) fall within permissible ventilation parameter range(s). Next, at step 2768, the VCD queries whether the request data passes the analysis. At step 2768, if the request data does not pass the analysis (e.g., at least one ventilation parameter fault mode condition is detected, or an alarm is triggered), then, at step 2770, the VCD controls the mechanical ventilation system of the respiratory distress ventilator according to last-received valid settings, back-up settings or settings determined by adjusting requested settings. However, at step 2768, if the request data passes the analysis (e.g., no ventilation parameter fault mode condition is detected), and does not trigger any alarms, then, at step 2772, the VCD controls in accordance with the requested settings. In some embodiments, for requested settings within certain ranges, user confirmation may be prompted and required before the VCD controls according to the requested settings.

FIG. 26 illustrates a block diagram 3050 of an example respiratory distress ventilator 3052 with one or more non-ventilation modes 3054 and one or more ventilation modes 3056. In the example depicted, when in the non-ventilation mode 3054, the respiratory distress ventilator 3052 may be used in patient monitoring and assessment, without providing any form of ventilation. In some embodiments, in the non-ventilation mode 3054, one or more spirometers may be used in assessing the patient's respiratory function. One or more non-ventilation modes 3054 may be used in emergency contexts, such as for a patient who is experiencing respiratory distress and requires monitoring but does not currently require mechanical ventilation. One or more non-ventilation modes may also be used in non-emergency contexts, such as for a patient who is not experiencing respiratory distress or who is at a non-emergency doctor's office or hospital visit. In some implementations, in a non-emergency care context, a respiratory distress ventilator may be used with a spirometer thereof, in assessing a patient's respiratory function or status. Such non-emergency care assessment may not include use of certain components that may be used in emergency care settings, such as patient circuits, particular patient circuits, or aspects of patient circuits. The respiratory distress ventilator may be capable of switching between non-ventilation and ventilation modes, such as by user selection, automatically, by user prompting and confirmation, or in other ways.

When in the one of the ventilation modes 3056, the respiratory distress ventilator 3052 may provide any of various forms of ventilation, examples of which are described with reference to FIG. 27, but may also provide or continue to provide patient monitoring and assessment.

FIG. 27 illustrates a block diagram of example respiratory distress ventilator ventilation capabilities or modes 3000, in accordance with some embodiments. As depicted, the respiratory distress ventilator 3002 includes ventilation capability 3004, of various types and/or modes. In some embodiments, for example, these include: (1) non-invasive ventilation (NIV) 3006, including continuous positive airway pressure (CPAP) 3008 and bilevel positive airway pressure (BPAP) ventilation 3010; (2) high flow nasal cannula (HFNC) 3012; (3) invasive ventilation (IV) 3014, including assist-control (AC) 3016, synchronized intermittent mandatory ventilation (SIMV) 3018; and (4) ventilation provided during CPR chest compressions 3022. Furthermore, in some embodiments, a respiratory distress ventilator may provide ventilation in pressure or volume targeted modes.

Generally, NIV 3004 has been increasingly used in acute care settings over the last two decades, particularly for hypercapnic respiratory failure. In particular, patients with acute hypercapnic respiratory failure, due to COPD or other etiologies, may respond well to NIV 3006. Furthermore, there is evidence that supports the use of NIV 3006 in treatment of hypercapnic respiratory failure of other etiologies as well. In some embodiments, a respiratory distress ventilator may be optimized to provide superior NIV 3006 in CPAP 3008 and bilevel 3010 modes.

In some embodiments, a respiratory distress ventilator may be provided with characteristics in providing NIV 3006 as follows. In providing NIV 3006, a respiratory distress ventilator may operate over a pressure range of at least from 0 to 50 cm H2O and may provide a maximum variation of, for example, plus or minus 0.3 cm H2O. Furthermore, in some embodiments, maximum pressure variations may be maintained as follows. At a pressure of less than 10 cm H2O, the following maximum pressure variations may be maintained: at 10 BPM, plus or minus 0.4 cm H2O; at 15 BPM, plus or minus 0.5 cm H2O; and, at 20 BPM, plus or minus 0.8 cm H2O. At a pressure of between 10 and 50 cm H2O, the following maximum pressure variations may be maintained: at 10 breaths per minute (BPM), plus or minus 0.5 cm H2O; at 15 BPM, plus or minus 0.8 cm H2O; and, at 20 BPM, plus or minus 1.0 cm H2O. Still further, in some implementations, a maximum flow rate of 250 liters per minute may be maintained over an operating range of pressures. However, other embodiments may be used with greater or lesser accuracies and variations and different characteristics.

In some embodiments, in modes of operation such as a NIV 3006 mode, various respiratory distress ventilator operating parameters may be increased gradually for reasons that may include maximizing or optimizing system performance, stability or efficiency (such as in connection with clinical concerns, or energy, oxygen or other resource usage), or patient safety or comfort. Additionally, in some embodiments, a portable ventilation or respiratory distress ventilator may be capable of detecting and compensating for detected leaks, such as inspiratory or expiratory patient circuit leaks, for similar reasons. In various embodiments, leak detection can be done in different ways. For example, leak detection may be accomplished by determining a difference between detected inspiratory and expiratory flows. Leak detection may also be accomplished by determining a declining PEEP at the end of expiration, as another example. Leak compensation can be accomplished, for example, by causing appropriate compensatory additional gas flow using a gas flow generator such as a blower. In some embodiments, a user may be able to fix the leak, and CSG may be used in guiding the user in this regard.

In some embodiments, in modes such as NIV 3006 or others, a respiratory distress ventilator may provide alarms (whether presented on or at the respiratory distress ventilator, or elsewhere, such as part of CSG on a display/GUI) that may relate, for example, to patient or ventilation related parameters, such as leaks or possible leaks, such as in connection with PEEP or expiratory positive airway pressure (EPAP), excessively high or low parameter values, such as SpO2 or other gas exchange related parameters, Vt, Ve or RR, as well as respiratory parameters such as Rrs, respiratory compliance (Crs) and others. Furthermore, in some embodiments, a respiratory distress ventilator, while providing bilevel ventilation, may vary parameters such as PIP or inspiratory positive airway pressure (IPAP), such as to ensure or help ensure that a target Vt is achieved. Additionally, in some embodiments, a respiratory distress ventilator may, for example, vary a baseline pressure in order to determine or help determine an optimal level of PEEP or EPAP to apply. Also, in some embodiments, a respiratory distress ventilator may detect or determine asynchrony or potential asynchrony related events, and alerts, alarms or CG may be determined and provided accordingly. Still further, in some embodiments, the respiratory distress ventilator may detect or determine trends over time, such as regarding parameters such as respiratory resistance (Rrs) and Crs or others, and may provide alerts, alarms or CSG accordingly. Furthermore, in some embodiments, a portable ventilation or respiratory distress ventilator may include a manual breath option or button.

In HFNC 3012, heated and humidified oxygen or oxygenated gas may be delivered to the nose at high flow rates. These high flow rates may generate low levels of positive pressure in the upper airways, and FIO2 may be adjusted accordingly, for example. The high flow rates may also decrease physiological dead space by flushing expired carbon dioxide from the upper airway, a process that potentially explains an observed decrease in the work of breathing in patients, with use of HFNC 3012. In patients with acute respiratory failure of various origins, high-flow oxygen has been shown to result in better comfort and oxygenation than other types of oxygen therapy. In particular, evidence has been developed supporting the use of HFNC 3012 in pediatric patients with mild to severe bronchiolitis. Study results suggest that patients treated using HFNC 3012 returned to eating sooner, required less oxygen, and were discharged from hospitals sooner than those who received other oxygen therapy.

It has been observed that HFNC 3012 can play a significant role in heating to a core temperature and humidifying patient's respiratory system, especially with high flow rates, where other oxygen delivery systems may fail or underperform. For example, HFNC may improve mucociliary function and secretion clearance, as has been evidenced in patients with idiopathic bronchiectasis, such as from observations of reduced percentage of retention of an inhaled tracer before and after receiving warm and humidified gas via HFNC. Additionally, it has been observed that, with HFNC, the required metabolic cost of warming and increasing the relative humidity of inspired gas is reduced, especially in patients with high spontaneous Ve. In some embodiments, a respiratory distress ventilator includes or is equipped with a heated humidifier. Furthermore, in some embodiments, alarms are provided that relate to heated humidifier operation.

In some embodiments, in providing IV 3014, a respiratory distress ventilator may provide the following parameter value ranges: RR in the range of 1 to 12 BPM—plus or minus 1 BPM, RR in the range of 12 to 50 BPM—plus or minus 2 BPM; Vt of 50 to 2000 ml plus or minus 15%+4 ml; PIP of 5 to 65 cm H2O, inspiratory time (TI) 0.3 to 3 plus or minus 10% seconds; peak flow of 200 plus or minus 10% liters/minute; PEEP of 5 to 30 cm H2O, trigger sensitivity of −2.5 to 10 cm H2O; pressure support (PS) of 5 to 50 cm H2O; and FiO2 of 21 to 100%. However, other embodiments may be used with greater or lesser accuracies and variations and different characteristics.

In some embodiments, the respiratory distress ventilator provides ventilation during CPR chest compressions, including ventilation provided during a period of time during which CPR chest compressions, or CPR chest compression therapy, is being provided. Ventilation provided during CPR chest compressions may be, for example, coordinated with, or synchronized with, the compressions. This may include, for example, determining the amount of breaths per minute (or other unit of time) based on, or based at least in part on, the number of compressions per minute (or other unit of time). Ventilation provided during CPR chest compressions may be provided asynchronously with the CPR chest compressions, such as where the number of breaths per minute (or other unit of time) is independent of the CPR chest compression rate. In some embodiments, ventilation during CPR (such as, for example, SIMV) is provided using pressure targeting, regardless of the airway state.

In some embodiments, a respiratory distress ventilator includes at least one spirometer. In various embodiments, various degrees, breadth, or levels of basic or advanced spirometry may be included.

In some embodiments, a goal of the limited spirometry may be to facilitate patient respiratory assessment in order facilitate screening for, or making an assessment associated with, a disease state or etiology. In some embodiments, for example, one or more respiratory parameters of a patient, determined or obtained using spirometry, may be used in making assessments or determinations with respect to disease states or etiologies, possible disease states or etiologies, or probabilities or probability ranges associated with particular disease states or etiologies. In some embodiments, values or ranges of particular respiratory parameters of the patient, determined or obtained using spirometry, may be compared with values or ranges associated with healthy patients, or patients with particular disease states or etiologies, or patients that are particularly likely to have particular disease states or etiologies. A respiratory status of the patient can include any of the foregoing, or data relating to any of the foregoing, among other things, including respiratory distress associated with another condition, which may be a respiratory or non-respiratory condition.

For example, in some embodiments, a respiratory distress ventilator may include spirometry that meets the American Thoracic Society (ATS)/Europea respiratory Society (ERS) Standard and International Organization for Standardization (ISO) 26782. The spirometer may be capable of spirometric volume detection in the range of 0 to 9 liters (or, e.g., up to 12, 15, 18 or 21 liters), flow detection in the range of 12 to 900 liters/minute (or, e.g., a lower limit of 2, 5 or 9, 15, 20, 25 or 30, or, e.g., an upper limit of 150, 200, 250, 300, 350, 400, 450, 500, 600, 700, 800, 850, 950, 1,000, 1,100, 1,200, 1,300, 1,400 OR 1,500), and an accuracy of plus or minus 2.5% (or, e.g., between 0.1% and 10%, such as 0.1%, 0.5%, 1%, 1.5%, 2%, 2.5%, 3.0, 3.5%, 4%, 4.5%, 5%, 6%, 7%, 8%, 9% or 10%).

In some embodiments, data or measurements relating to the patient, determined or obtained using spirometry, may be compared against reference or predicted values based on characteristics of the patient. This may be done in connection with determining whether a patient has or may have a particular, significant or severe disease state or etiology, or in connection with determining an associated probability or probability range. For example, measurements within, or far enough within, normal (or other) ranges may suggest or evidence the absence of such disease states or etiologies. However, measurements outside, or far enough outside, normal (or other) ranges may suggest or evidence the presence of such disease states or etiologies. In some embodiments, data or measurements relating to the patient, determined or obtained using spirometry, may be compared with other reference spirometric data, such as spirometric data averages or data associated particular disease states or etiologies, types or groups thereof, or probabilities associated therewith. This may be done to facilitate patient disease state or etiology assessments or determinations. Also, in some embodiments, spirometric data or measurements relating to the patient may be compared with reference spirometric data associated with particular types, groups, or demographic or physiological characteristics of individuals (e.g., age, gender, ethnic background, current or historical health, health problem or disease history, etc.). Similarities or differences between the characteristics of the patient (or patient related data) and the characteristics of the individuals associated with the reference data (or other aspects of the reference data), may be taken into account in the patient disease state or etiology assessments or determinations. In some embodiments, various algorithms, machine learning or artificial intelligence may be used in facilitating the patient disease state or etiology assessments or determinations.

It has been shown that hyperventilation can cause or be associated with, for example, increased intrathoracic pressure, impaired venous return, decreased coronary perfusion pressure, and lower patient survival rates. As a result, in some embodiments, a respiratory distress ventilator may be used in providing, for example, modest/conservative, consistent ventilation during CPR chest compressions. In some embodiments, for example, during masked ventilation without airway protection, a respiratory distress ventilator may deliver synchronized ventilation, e.g., 2 (or, e.g., 1-5) breaths for every 30 (or, e.g., 10-100) compressions. Furthermore, in some embodiments, for example, during ventilation with airway protection (e.g., via endotracheal tube or esophageal tracheal airway), a respiratory distress ventilator may deliver, e.g., 10 breaths/min (or, e.g., 5-20) independently of the compression rate (asynchronously).

FIGS. 28A-C illustrate a few of many possible variations of respiratory distress ventilator included controls that may be used in various embodiments. In FIGS. 28A-C, suggestive respiratory distress ventilator outlines and shape are shown, and detail is omitted on the tops and sides. In various embodiments, the degree of included controls may vary along spectrums of inclusiveness and complexity. More limited sets of included controls may emphasize or favor, for example, aspects such as simplicity of operation, smaller size, or greater reliance on remote control. More inclusive sets of included controls may emphasize or favor, for example, greater user information, greater control available at the respiratory distress ventilator and less reliance on remote control. In some embodiments, the particular included features in a set of included controls may be optimized based on various factors, such as may relate to, for example, anticipated or likely use or uses, user training or experience, degree of available user attention to the respiratory distress ventilator, or other factors. In some embodiments, aspects of the included sets of controls 4002, 4022, 4042 as depicted in FIGS. 28A-C may be used in providing user interactive CSG, examples of which are described with reference to FIGS. 30-35. In some embodiments, one or more remote control devices may have sets of controls that are optimized based in part on the included controls of the respiratory distress ventilator, since the devices may operate in an integrated fashion and environment.

FIG. 28A illustrates an example front of a respiratory distress ventilator 4000. The respiratory distress ventilator 4000 has included controls 4002 including only a power switch 4004 and communication indicator light 4006 (or, in other embodiments, the communication indicator light could be, e.g., a remote control indicator light). The communication indicator light 4006 (or remote control indicator light) may be lit when communication with a remote control device or system is present (or, if a remote control indicator light, when remote control is occurring), or to indicate, for example, wired connection, wireless connection, Bluetooth connection, or one or more other forms or protocols of connection.

In embodiments such as those depicted in FIGS. 28A-C, a display/GUI of the respiratory distress ventilator 4000 may include, e.g., a liquid crystal display (LCD), organic LED (OLED), microLED, or other type of display. In some embodiments, the display/GUI may include touchscreen aspects. Furthermore, in some embodiments, the display/GUI may be user selectively hidden or displayed, and, when hidden, may provide some indication, such as a visible or flashing light, if user attention is needed.

In some embodiments, the display/GUI of the respiratory distress ventilator 4000, or one or more other portions or components of the respiratory distress ventilator 4000, may be modular, and may be attachable to and removable from the respiratory distress ventilator 4000, such as an attachable and detachable LCD display module. Furthermore, in some embodiments, various different types of display modules may be attached to the respiratory distress ventilator 4000, such as display modules of varying complexity, so that the respiratory distress ventilator 4000 may be customizable by a user with regard to the display/GUI. Furthermore, in some embodiments, the display/GUI of the respiratory distress ventilator 4000 may itself be modularized and include multiple modular components, such as a top, middle and lower components, for example. Any of the components may be attachable and detachable, allowing mixing of different display components and associated customization of the overall display/GUI of the respiratory distress ventilator 4000.

In some embodiments, patient circuits of, or coupleable with, the respiratory distress ventilator 4000 may be configured such that all portions of the patient circuits are attached, and they attach as a modular unit to the respiratory distress ventilator 4000.

Furthermore, in some embodiments, the respiratory distress ventilator 4000 may include a handle or through-hole, for example, allowing convenient or stable grasping or manipulation of the respiratory distress ventilator 4000. In some embodiments, a handle may itself include a display, which may include status or alarm indication areas or lighted areas, for example.

FIG. 28B illustrates an example front of a respiratory distress ventilator 4020, with included controls 4022 including a power switch 4024, communication or remote control light 4026 (which features may, for example, be similar to the corresponding features 4004, 4006 as depicted in FIG. 28A), and a display/GUI area 4028

The display/GUI may include a central display/GUI area 4032, up and down arrow selection buttons 4030 and plus and minus selection buttons 4034. In the embodiment shown, a user can use the up and down arrow selection buttons 4030 to scroll through multiple items for display, some or all of which may be subject to potential setting or adjustment by the user. Scrollable items may include, for example, a mode or modes of operation (e.g., one or more non-ventilation modes or one or more ventilation modes, such as those described with reference to FIGS. 26 and 27), and various parameter settings, such as patient physiology related parameter settings or ventilation related parameter settings, among other things. In the example shown, the current FiO2 setting of the respiratory distress ventilator 4020 is shown in the central display/GUI area 4032 (no actual setting is shown, but, in some embodiments, FiO2 may vary between, e.g., 21% and 100%). As an example, a user may have used the up and down arrow selection buttons 4030 to select the FiO2 parameter setting for display. Once displayed, the user may be able to change the displayed current setting by using the plus and minus selection buttons 4034 to increase or decrease it (potentially subject to potential limits, confirmation, etc.). In various embodiments, the changed displayed setting may be implemented without further indication to the user (potentially unless user confirmation is required), or some indication to the user may be provided (e.g., the display/GUI may momentarily blink or change color, or some audio confirmation may be provided, such as a sound or one or more words).

FIG. 28C illustrates an example front of a respiratory distress ventilator respiratory distress ventilator 4040, with included controls 4042 including a power switch 4044 and communication or remote control indicator light 4046, which may be similar to those 4004, 4024, 4006, 4026 depicted in FIGS. 28A-B, for example. The included controls 4042 also include a display/GUI area 4055 and plus and minus selection arrows 4056, which may be, in some ways, similar to the central display/GUI area 4032 and plus and minus selection arrows 4034 as depicted in FIG. 28B. However, in the embodiment depicted in FIG. 28C, no up and down arrows are provided. In this embodiment, instead of using up and down arrows to select an item for display (and potentially for the user to change a setting), a dial 4050 is provided. In some embodiments, for example, the user may turn the dial 4050 clockwise or counterclockwise to scroll through and change the currently displayed item in the display/GUI area 4055 (or other areas of the display/GUI) and may press the dial in to make certain selections, adjustments, changes, confirmations. In some embodiments, a scroll wheel may be included, allowing, for example, scrolling to choose a displayed item and pressing to select the item, such as to choose or dismiss an alarm, for example. In various embodiments, however, many variations may be used with regard to sets of included controls, such as different breadths and size of a set of included controls, particular types and items of controls, and different ways in which a user may interact or provide different forms of input.

In the embodiment depicted in FIG. 28C, a larger top display/GUI area 4048 displays various information. The top display/GUI area 4048 includes display of a current mode of operation, such as a current type of ventilation that is being provided by the respiratory distress ventilator respiratory distress ventilator (examples of types of ventilation that may be provided are described with reference to FIG. 27). The top display/GUI area also includes display of various patient and ventilation related parameters. These include, on the left side, HR, BPM and SpO2, and, on the right side, FiO2, PEEP, PIP and Vt. Sets of double arrows 4058 are shown next to the values for FiO2 and PEEP. These may indicate that FiO2 CLC and PEEP CLC are currently on/operating. In various embodiments, if FiO2 CLC or PEEP CLC was not currently on/operating, the corresponding sets of double arrows might not appear, or might appear but include a visible cross out, for example, to provide an appropriate visual indication to the user.

In a central area of the included controls 4042, an alarms/alerts display/GUI area 4052 and a message area 4052 are provided. The alarms/alerts display area 4052 may be used for display of any of various alarms or alerts for the user, such as may relate to concerning or unsafe patient or ventilation conditions or parameters. In some embodiments, displayed alarms may be coded or color coded, such as may indicate the priority or urgency of the alarm. For example, alarms displayed in green, yellow or red colors, or including associated green, yellow or red displayed lighted areas, may indicate increasing levels of priority or urgency. The message area 4054 may be used for display of any of various messages for the user, such as may include CSG, or may provide additional information relating to a provided alert or alarm, for example. In some embodiments, user scrolling may be used (such as using the dial 4050) to scroll through and change the currently displayed item in the alarms/alerts display/GUI area 4052 or the message area 4054. For example, in some embodiments, the user may be able to scroll up and down through a list of saved alerts or alarms, or a list of saved messages.

In some embodiments, a respiratory distress ventilator 4102, an example of which is provided in FIG. 1, may provide mechanical ventilation incorporating one or more forms of CLC, such as FiO2 CLC or PEEP CLC. In various embodiments, the CLC (or one or more forms) may be implemented via remote control by one or more remote control devices or systems, or via control by the respiratory distress ventilator, or with aspects of both. In some embodiments, control including CLC may or may not be subject to user interaction or include CSG. In various embodiments, if user interaction is included, it may take place at any of various devices, such as the respiratory distress ventilator 4102, a remote control device or another device or system in the environment.

The term CLC, as used herein, may refer to control of one or more ventilation related or patient related parameters, such as with relatively little or no required user action, participation or intervention, and can include reference to, but is not limited to reference to, fully automated or fully automatically regulated control. CLC may include, for example, device facilitated or algorithmically facilitated tracking, control and adjustment of one or more parameters, which may or may not include user involvement or participation. Where user involvement or participation is included, it may include, for example, confirming a suggested or recommended ventilation setting change or configuration, deciding on implementing a course of action, selecting one of several suggested courses of action, responding to a presented alert or alarm, or other decisions, choices or actions. User involvement or participation could also include, for example, setting or changing a parameter, where a closed loop control algorithm proceeds from there, initially according to the user-set or user-changed parameter setting. In various embodiments, if there is user involvement, it may be, for example, among other things, in whole or in part user-initiated, or in whole or in part prompted, suggested, recommended or required.

In some embodiments, closed loop control may be utilized but may be subject to manual adjustment or override by the user. For example, in some embodiments, although FiO2 and PEEP may be algorithmically and automatically controlled, a user may be able to intervene and manually change the FiO2 and/or PEEP setting. In some embodiments, following any manual adjustments, closed loop control of FiO2 and/or PEEP may resume from that point, at least until any further manual adjustments are made.

In various embodiments, CLC may include FiO2 being continuously adjusted based on measured patient SpO2, which is used as an indicator of patient oxygen saturation. Generally, a lower SpO2, or decreasing SpO2 (as may be determined, for example, based on a single or current SpO2 measurement, or several SpO2 measurements over a recent period of time) may tend to indicate a “sicker” patient, or patient with more impaired lung or respiratory system function, who is in need of more oxygenation support. In some embodiments, generally, when a patient's SpO2 is below a target level (such as a pre-determined SpO2 value), and potentially also based at least in part on how much below, and/or decreasing, FiO2 may algorithmically tend to be increased in order to increase support of a patient's oxygenation. When SpO2 is above the target level, and potentially also based at least in part on how much above, FiO2 may algorithmically tend to be decreased, since the patient may not be in need of as much oxygenation support. As such, FiO2 may be increased or decreased based at least in part on the need of the patient as indicated at least in part by SpO2 and/or, for example, some other indication(s), such as one or more other non-invasively sensed, measured or determined indications of patient oxygen saturation. Algorithmically determined factors, such as derivative gain and proportional gain factors, may affect a direction (increase or decrease) and amount of FiO2 adjustment.

In some embodiments, one or more previously measured SpO2 values may be taken into account algorithmically in determining an FiO2 adjustment. In particular, in some embodiments, proportional gain and derivative gain may take into account one or more previously measured SpO2 values. Generally, in some embodiments, derivative gain may take into account an effective rate of decrease in SpO2 for some period of time up to and including the time of the currently measured SpO2 (such as may include use of a moving average). Derivative gain may tend to increase the FiO2 adjustment when SpO2 is decreasing, such as in a manner that may be associated with, or proportional to, a determined rate of SpO2 decrease. However, in some embodiments, derivative gain may not operate to tend to decrease FiO2 when SpO2 is increasing, and may in this regard be asymmetric in operation. Furthermore, in some embodiments, proportional gain may take into account a determined magnitude of the difference between the current (or current and recent) SpO2 and a target SpO2, where a larger difference may lead to a greater adjustment. Embodiments of algorithms for closed loop control of FiO2, including use of derivative gain and proportional gain, are described with reference to FIG. 12A.

In some embodiments, PEEP is adjusted based at least in part on FiO2, but also based at least in part on the current PEEP. Since FiO2 may be adjusted based at least part on patient SpO2, and since SpO2 may be associated with how “sick,” compromised or functionally impaired the patient's lungs or respiratory system are, conceptually, FiO2 may to some degree represent or effectively operate as a surrogate or indicator of how “sick” the patient's lungs or respiratory system are. For example, in some instances, a relatively low and/or decreasing SpO2 may indicate substantial and/or increasing degree of functional lung impairment, and may result in a relatively high FiO2. This high FiO2 may, under appropriate circumstances, warrant and lead to an increase in PEEP (as may be determined with reference to, for example, PEEP change eligibility rules and PEEP selection rules), where PEEP may help essentially open alveoli and support respiratory function. Conversely, in some instances, a relatively high and/or increasing SpO2 may indicate less substantial and/or decreasing lung impairment (i.e., generally, the patient may be breathing “better” on their own), and may lead to a relatively low FiO2, which may, in some instances, warrant and lead to a decrease in PEEP (as may be determined with reference to, for example, PEEP change eligibility rules and PEEP selection rules).

As such, adjusting PEEP based at least in part on FiO2 may result in PEEP being adjusted based at least in part on an indication of how “sick” or functionally impaired the patient's lungs or respiratory system may be.

PEEP can support a patient by, for example, increasing alveolar pressure and volume, which can essentially distend and prevent the collapse of alveoli, improving oxygenation. However, too high a PEEP can cause risk to a patient. For example, too high a PEEP can lead to an increased thoracic pressure, which in turn can lead to a decrease in patient blood pressure by inhibiting or otherwise limiting venous blood return, causing low blood pressure related risk to the patient. Also, since it can take some time for an increased PEEP to have full effect on alveoli, the full effect of an increased PEEP may take some time, such as minutes, to fully emerge. That can be a reason to avoid increasing PEEP too rapidly, since there may be a possibility of essentially “overshooting the mark” in terms of PEEP increase rapidity and creating patient risk. Furthermore, overly high or over-frequent changing of the PEEP can create other risks to the patient, such as risk of barotrauma, damaging or rupturing alveoli, pneumothorax, pulmonary interstitial emphysema, pneumomediastimum, fibrogenesis, inflammation or a damaging immune response. As such, while it can be important to increase PEEP where warranted, in can also be important to avoid increasing PEEP too much or too frequently. In some embodiments, algorithms including PEEP change eligibility rules and PEEP selection rules provide an optimized approach to PEEP control, balancing assessed need for PEEP adjustment with need for avoiding too high a PEEP and avoiding over-frequent changes in PEEP. A more conservative or slower approach to decreasing PEEP may be warranted, relative to increasing PEEP, since decreasing PEEP is not undertaken in order to address a need of the patient for increased support.

In some embodiments, various parameters in addition to FiO2 and PEEP may be continuously adjusted or adjusted using closed loop control. These may include, for example, adjustments that may be based at least in part on EtCO2, which may indicate or suggest normocapnia, hypercapnia or hypocapnia. For example, in some embodiments, parameters including Vt, Ve, PIP, and driving pressure or plateau pressure may be continuously adjusted.

While embodiments of the present disclosure are applicable to both hospital and non-hospital settings, some embodiments may be particularly advantageous in non-hospital, out-of-hospital or field settings, such as for use with, or as, a respiratory distress ventilator to be operated or overseen by a user or care provider with limited ventilation related training or experience. In some embodiments of CLC as described herein, by reducing necessary user monitoring or control of various ventilation or patient related parameters, while yet optimizing such parameters, patient care, safety, outcomes and even survival rates can be significantly improved, particularly in pre-hospital situations. In some embodiments, one or more initial ventilation settings may be determined or optimized based at least in part on patient data, which may be at least in part user provided, such as the gender and height, or estimated height, of the patient.

In some embodiments, during ventilation of a patient, an oxygen concentration of the patient's blood, such as SpO2, or another patient oxygenation related parameter, is continuously measured, determined and tracked. Based at least in part on the determined oxygen concentration of the patient's blood, an FiO2 setting of the ventilator, or other oxygen related setting that may be associated with provided breathing gas, is appropriately adjusted, such as may include appropriate actuation of an oxygen source valve, actuation or adjustment of one or more other valves or settings associated with an oxygen source or concentrator, or in other ways. Based at least in part on the adjustment to the FiO2 setting, a PEEP setting of the ventilator is appropriately adjusted, such as may include appropriate actuation of an exhalation valve, actuation or adjustment of one or more other valves or settings, or in other ways.

The monitoring of the oxygen concentration of the patient's blood, checking/updating of the FiO2 setting and checking/updating of the PEEP setting may be performed on a very frequent basis, such as, e.g., every fraction of a second, every second, every more than one second or several seconds, every minute or several minutes, or based on irregularly or varying duration periods. In various embodiments, FiO2 may be adjusted at the same frequency as PEEP, or they may be adjusted at different frequencies. Monitoring and adjusting of parameters may be implemented or facilitated by one or more controllers that may execute one or more appropriate algorithms stored in one or more memories. Additionally, optimized initial settings may be determined and utilized.

In some embodiments, PEEP selection rules are provided. According to some embodiments, a PEEP setting may be changed only if an FiO2 setting changes so as to fall outside of an FiO2 range associated with the current PEEP setting. Moreover, in some embodiments, each of a number of possible PEEP settings is associated with a particular FiO2 range, but FiO2 ranges, or adjacent FiO2 ranges, may overlap. As a result, the current PEEP setting is maintained unless the FiO2 setting changes not only (a) enough to fall into a portion of the current FiO2 range that overlaps with another FiO2 range associated with a different PEEP setting, but (b) enough to go beyond the overlapping portion as well as into an FiO2 range associated with a different PEEP setting. Generally, requiring FiO2 change beyond the applicable range, as described in embodiments herein, in order to change the PEEP setting (whether an increase or decrease) can effectively limit the rate or frequency of change of PEEP, balanced with the need to change PEEP over time when warranted. This, in turn, can have advantages such as allowing more time for a previous PEEP change to have full effect (since a PEEP change takes some time to have full effect on alveoli) before changing PEEP. It can also have the advantage of avoiding abrupt or over-frequent PEEP oscillations (which can disrupt, break open, or otherwise damage alveoli) and achieving overall throttling or smoothing of PEEP change over time, for example.

In some embodiments, the above can be viewed as creating an intended, throttled or balanced tendency to maintain, rather than change, the current PEEP setting, while yet indicating PEEP change under appropriate circumstances. In some embodiments, this, in turn, can improve patient safety and outcome, given that, for example, while PEEP setting changes are necessary under appropriate circumstances, overly frequent, sudden and/or large changes in PEEP setting may cause serious risk to the patient, since over-frequent PEEP changes can break apart alveoli and damage a patient's lungs, for example. Therefore, it can be optimal to appropriately balance various factors, which can be accomplished by some embodiments disclosed herein.

Furthermore, in some embodiments, possible or allowed PEEP settings are determined to include a discrete set of particular PEEP settings, or levels, each of which is associated with an FiO2 range, where adjacent ranges may overlap. In some embodiments, a PEEP setting can only be adjusted so as to change at most by one level up or down, even if, for example, the FiO2 setting changes dramatically.

Additionally, some embodiments provide or utilize particular rules or conditions, or PEEP change eligibility rules or conditions (and/or ineligibility rules or conditions), which must be met in order for a PEEP setting change to be allowed and actually made, even if, for example, it may be indicated by the PEEP selection rules. In some embodiments, even if a PEEP setting might otherwise be indicated by the PEEP selection rules, unless and until the specified eligibility conditions are met (or ineligibility conditions avoided), PEEP is be maintained at the current setting. In some embodiments, for example, these conditions or rules provide an appropriate or optimal balance of factors affecting performance and safety, such as by balancing the advantage of appropriate change and rate of change with the advantages of, for example, appropriate relative stability and limited rate of change.

Generally, in some embodiments, the time periods required for PEEP change eligibility may reflect a balance of the advantages of stability relative to the need for change, as described above. In some embodiments, the PEEP change eligibility rules may include that PEEP has not been changed in at least a specified period of time, and the specified period of time may be different depending on whether a PEEP increase or decrease is being evaluated. Furthermore, in some embodiments, the specified period of time for a PEEP increase (e.g., in minutes, between 2-3, 3-4, 4-5, 5-7, 7-10, 10-15, 15-20, 20-25, 25-30, 30-35 or 35-40) may be less than the specified period of time for a PEEP decrease (e.g., in minutes, between 10-20, 20-30, 30-40, 40-50, 50-60, 60-70, 70-80, 80-90, 90-100, 100-110 or 110-120). Reasons for this may include that, if a PEEP increase is indicated, that can mean that the patient's lungs are becoming more “sick” or functionally impaired, which may warrant relatively fast action to change PEEP in order to provide the patient with more support, whereas decreasing PEEP does not increase patient support, and so a longer period of time for a PEEP decrease may be warranted.

Furthermore, in some embodiments, PEEP change eligibility rules may include that the FiO2 setting has not changed, or has not changed by at least a particular amount, during a first period of time (which can be termed, for convenience a “steady state” situation with regard to FiO2), or the patient has been in what may be termed a desaturation condition for at least a second period of time, such as may be indicated by patient SpO2, as described with reference to FIG. 13. In some such embodiments, PEEP can only be changed, even if a PEEP change is otherwise indicated, if at least one of two conditions are met: (1) the FiO2 has been steady state (no change in FiO2, or no change beyond a certain minimal threshold) for the first period of time (e.g., in seconds, 0-15, 15-30, or 30-60, or, in minutes, 1-2, 2-5, 5-10, 10-12, 12-15, 15-20, 30-45 or 45-60), or (2) the patient has been in a desaturation condition (e.g., SpO2 of at or below, e.g., 85%, 86%, 87%, 88%, 89%, 90%, 91% or 92%) for at least a second period of time (e.g., in seconds, 0-15, 15-30, or 30-60, or, in minutes, 1-2, 2-5, 5-10, 10-12, 12-15 or 15-20).

In some embodiments, conditions (1) and (2) may prevent allowing for a potential PEEP change under conditions where the FiO2 suddenly changes, or seems to suddenly change, for reasons that might not reflect the correct patient conditions under which to allow change of the PEEP setting, such as, for example, as may result from erroneous or inaccurate measurement or determination of FiO2. However, condition (2) may include a smaller time period requirement in order to allow for a potential PEEP change to provide an enhanced or prioritized ability to rapidly respond to patient desaturation, which can indicate a critical and urgent emergency warranting urgent increased patient support, which may include a PEEP increase, for example.

In some embodiments, PEEP change eligibility rules may include that, for a PEEP increase, the PEEP has not changed for at least a first period of time, and for a PEEP decrease, that the PEEP has not changed for at least a second period of time. The first and second periods of time may be different. As such, in some embodiments, particular time thresholds are set, which thresholds must be met in order to increase or decrease PEEP, even if a PEEP increase or decrease is otherwise indicated. Furthermore, the thresholds may differ based on whether the indicated PEEP change is an increase or a decrease. As such, in some embodiments, PEEP selection rules, or aspects thereof, may be referenced as part of or related to use of PEEP change eligibility rules.

Additionally, in some embodiments, PEEP change eligibility conditions may include that one or more measures of the patient's hemodynamic status, as may, for example, be determined at least in part based on a blood pressure measurement, is of at least a predetermined level, which blood pressure measurement may, in various embodiments, be obtained by the ventilator or another system or device in communication with the ventilator, such as a separate critical care monitor, defibrillator, or other device, for example. In some embodiments, this condition may ensure that the patient's hemodynamic status is sufficient to warrant a change in PEEP.

FIG. 29 illustrates a block diagram of ventilation control software 300 including CLC capability. In various embodiments, the software 300 may be stored in whole or in part in the one or more memories of, and may be executed by one or more processors of, one or more controllers. The one or more controllers may include a controller of a respiratory distress ventilator, or a controller of one or more remote control devices or systems for remote control of a respiratory distress ventilator, for example.

The ventilation control software 300 includes components including an initiation mode 302, a test breaths mode 304 and a ventilation mode/active mode 306, which may represent, for example, software architectural or conceptual components. Various other software may be included.

The active mode 306 includes ventilation control 310, FiO2 control 312 and PEEP control 314, where each or some of these aspects 310, 312, 314 may be in coordination with a central control aspect 308 or operate independently, and each may or may not also be in coordination with each other or some of the other aspects. Different aspects of CLC may be activated alone or in combination. For example, in some embodiments, FiO2 closed loop control is activated without PEEP closed loop control, such that PEEP is manually adjusted by the user; or PEEP closed loop control is activated without FiO2 closed loop control, such that FiO2 is manually adjusted by the user; or ventilation closed loop control may be activated where FiO2 and PEEP are based manually adjusted by the user.

In some embodiments, FiO2 closed loop control can provide advantages including minimizing the time that a patient's SpO2 is below or substantially below a target value, providing rapid oxygenation response to patient SpO2 desaturation events and helping prevent patient hypoxemia and hyperoxymia.

It is noted that, while the modes and control aspects are depicted separately for conceptual purposes, they may be implemented in a combined, integrated or different manner, such as embodiments in which aspects of the roles of each of the modes or control aspects are distributed differently or combined in various ways. Moreover, other conceptual frameworks may also be utilized in various embodiments.

In some embodiments, a portable ventilation or respiratory distress ventilator respiratory distress ventilator may be operated (by the respiratory distress ventilator, or by a remote control device) in one of several overall system modes of operation. One (or several) such system modes may include closed loop control of FiO2 and PEEP, which, for convenience and without limitation, may be referred to herein as ventilation/PEEP/FiO2 controller (VPFC) system mode. In some embodiments, operation in VPFC system mode includes initiation mode 302, test breaths mode 304 and active mode (or ventilation mode) 306, which modes may, in some embodiments, occur in order and represent phases of operation in VPFC system mode.

In various embodiments, various types of closed loop control, such as VPFC mode, can be started or engaged, as well as stopped or disengaged, in different ways. In some embodiments, VPFC or other aspects of closed loop control may start automatically, such as at the onset of active mode ventilation. In some embodiments, the respiratory distress ventilator or remote control device may include a control aspect, such as a physical button, control knob or GUI aspect, which the user may press, engage or actuate in order to start, select or turn on VPFC or a particular aspect of closed loop control. For example, in some embodiments, the user may be provided with options to select and initiate, for example, VPFC, closed loop control of FiO2 but manual PEEP control or adjustment, or closed loop control of PEEP but manual FiO2 control or adjustment. Furthermore, in some embodiments, the user may be provided with options to start, stop or re-start various types of closed loop control at different times, so that VPFC or other aspects of closed loop control are available, but also subject to disengagement, and so are made to be essentially on-demand. Also, as mentioned previously, in some embodiments, during operation of aspects of closed loop control, the user may be able to change a particular setting or settings, such as FiO2 or PEEP, and then closed loop control may resume from that point and initially with that setting or those settings. Furthermore, in some embodiments, a user may be provided with an option to stop or disengage VPFC or some other aspect of closed loop control, and later the user may restart it. Still further, in some embodiments, multiple or different users may oversee or manage operation of the respiratory distress ventilator, such as at different times, and each user may take different actions.

In some embodiments, for example, a user, such as a minimally trained user, may initiate active mode ventilation in VPFC mode (or another aspect of closed loop control). At some point during active mode ventilation, that user or another user, such a more trained user, may change from VPFC (or another aspect of closed loop control) to manual, or more manual, operation (whether via the respiratory distress ventilator or a remote control device). Alternatively, active mode ventilation may be initiated without VPFC or some other aspect of closed loop control, and VPFC or some other aspect of closed loop control may be started or engaged later, such as if the user becomes distracted (or during the period of user distraction), or by a less trained user who may later arrive at the scene, for example.

The following provides an exemplary overview of operation in VPFC system mode, according to some embodiments. In some embodiments, at the start of initiation mode 302, or after the user selects VPFC system mode, the user may be prompted (via a display, such as at the respiratory distress ventilator or a remote control device) to enter the patient's gender and height. From this, the controller (of the controlling device, such as the respiratory distress ventilator, a remote control device, or another device or system) may determine an associated bodyweight, which may be called the patient's “predicted” bodyweight (PBW). However, the determined bodyweight may not be the patient's actual bodyweight, and may represent an ideal bodyweight, approximated, typical or other associated bodyweight to be used, for example, in determination or optimization of certain ventilation settings. Predicted bodyweight may be used, as opposed to actual bodyweight, for example, since actual bodyweight may fluctuate substantially from person to person, and even between individuals of similar characteristics such as height and gender, due to different amounts of muscle and fat from person to person, while lung volume, capacity and function remain relatively unaffected by such factors. As such, in some embodiments, PBW may be used at least in part as an indicator associated with an individual's respiratory capacity, and may generally be a better indicator than actual patient bodyweight.

During initiation mode 302, the controller may utilize the PBW in determining one or more ventilation parameter settings to be used for one or several operational breaths to be delivered to the patient during initiation mode, which may be utilized, for example, in confirming correct operation of the respiratory distress ventilator 4102, which may include confirming that there are no leaks, or significant leaks, in the circuit. For example, PBW may be used in setting initial Vt, such as may be set as Vt (in mL)=A*PBW, where A may be, e.g., 5, 6, 7, 8 or 9. Furthermore, for example, initial Ve (in mL/min) may be set as B*PBW, where B may be, e.g., 80, 90, 100, 110 or 120.

Once initiation mode 302 is successfully completed, the respiratory distress ventilator 4102 may proceed to test breaths mode 304. During test breaths mode 304, one or more test breaths are delivered to the patient and respiratory data is obtained. The respiratory data is used in determining one or more patient respiratory parameters, such as estimated patient respiratory system compliance (Crs) and, in some embodiments, one or more additional parameters, such as estimated patient respiratory system resistance (Rrs). The determined patient respiratory compliance (Crs) is then used in determining an initial peak inspiratory pressure (PIP(0)) setting to be used at the start of active mode 306.

In some embodiments, active mode 306 may include closed loop control of FiO2 and PEEP, and/or possibly one or more other parameters. During active mode 306, central control 308, in coordination with ventilation control 310, FiO2 control 312 and PEEP control 314, sets or adjusts ventilation settings including FiO2, PEEP, PIP, target tidal volume (Vt), target minute volume (Ve), respiratory rate (RR), inspiratory:expiratory ratio (I:E). However, in some embodiments, one or more of ventilation control 310, FiO2 control 312, PEEP control 314, or other aspects, may set or adjust, or participate in the setting or adjusting of, certain of those or other ventilation settings.

In some embodiments, the controller continuously monitors for autoPEEP (PEEPi), which may result from incomplete emptying of the lungs when an expiratory phase is too short, and may create risk of dynamic hyperinflation of the lungs. In some embodiments, PEEPi is detected and measured based on detection and measurement of a gas flow existing at the end of an exhalation period. When PEEPi is detected, the controller may, in some embodiments, continuously adjust I:E to increase expiratory, or may decrease Vt/kg, which can increase expulsion of gas in order to eliminate PEEPi.

As described in detail herein, the controller may utilize a target SpO2, such as 94% (however, in various embodiments, the target SpO2 may be, e.g., between 93%-95%, between 93%-96%, between 93%-97%, between 93-98%, between 93%-99%, between 94%-95%, between 94%-96%, between 94%-97%, between 94%-98%, between 94%-99%, or another suitable range). On a continuous basis, actual measured patient SpO2 may be used as input in adjusting the FiO2 setting, which changed FiO2 setting or adjustment may further be used in adjusting the PEEP setting.

In some embodiments, determined parameters including patient end-tidal carbon dioxide (EtCO2), a determined patient airway pressure waveform (P), patient airway flow waveform (Vdot), and volume waveform (V) may be used in determining active mode initial settings for Vt, Ve, PIP, RR and I:E, and/or, in some embodiments, parameters can be used that are derived at least in part from the Vdot and V waveforms, which can include plateau pressure, driving pressure, respiratory compliance (Crs) and Rrs. Furthermore, the controller may continuously use determined patient respiratory compliance (Crs) and respiratory resistance (Rrs) in determining adjustments to Vt, such as may maintain Vt at a safe level. The controller 202 may also continuously adjust RR, such as to maintain Ve at a constant level when Vt is adjusted, where Ve=Vt*RR.

During active mode 306, the controller may cause delivery of ventilation to the patient using ventilation parameters that are continuously updated, which may include FiO2 and PEEP. It is to be understood that other embodiments do not use the particular exemplary modes described herein.

In initiation mode 302, the user may be prompted to enter the patient's gender and height, which may be used in determining the patient's PBW. Based on the determined patient's PBW, several ventilation parameter settings may be determined for use in test breaths mode 304.

In various embodiments, in initiation mode, various calculations may be utilized to determine test breaths parameter settings. In one example, in porcine use and studies (examples of which are provided herein with reference to later figures), Vt (in ml) may be calculated as 10*actual weight in kg, Ve (in ml/min) as actual weight in kg*140, and RR as Ve/Vt, where RR may be rounded to the next higher integer value. However, various other calculations and values may be used in various embodiments.

In another example, in human use, the following calculations may be utilized. The PBW (in kg) may be calculated as C+D*(height in cm−E), where C may be, e.g., between 35-60, D may be, e.g., between 0.80 and 1.00, and E may be, e.g., between 140-160, and where, in some embodiments, at least C may be slightly lower for a female than for a male. For Vt greater than or equal to, e.g., a value between 150-250 ml, rounding may be performed to, e.g. the nearest 3-7 ml, and for Vt less than or equal to, e.g., a value between 15-200 ml, rounding is performed to, e.g., the nearest 0.5-3 ml. However, various other calculations and values may be used in various embodiments, such as various calculations that are based on, or functions of, height, gender and/or one or more other patient physical or health-related characteristics.

In some embodiments, some or all of the results of the calculations made in initiation mode 302 are displayed to the user. The user may, for example, be prompted (e.g., at the respiratory distress ventilator or remote control device) to accept the determined parameters and then couple the patient to the ventilator.

In some embodiments, following completion of initiation mode 302, the controller proceeds to test breaths mode 304. In test breaths mode 304, the system may deliver one or several ventilation test breaths, based on which the system may determine one or more patient parameters and/or particular ventilator settings to utilize at the start of active mode 306. For example, in some embodiments, in the test breaths mode 304, a patient Crs, such as an estimated patient Crs, is determined and used in determining a PIP(0) ventilator setting. In some embodiments, respiratory compliance (Crs) may be calculated or estimated using patient respiratory dynamics data and the equation of motion for the respiratory system. Furthermore, in some embodiments, in test breaths mode 304, the following particular parameter settings may be utilized: I:E=1:3, PEEP=5 cm H2O, FiO2=0.5 (or 50%). However, in various embodiments, other initial settings may be used.

Following successful completion of the test breaths mode 304 and determination of the patient Crs, the controller may proceed to active mode 306. In some embodiments, the system may proceed to active mode 306 even if patient respiratory compliance (Crs) cannot be determined. For example, in some embodiments, if respiratory compliance (Crs) cannot be determined, a default respiratory compliance (Crs) value may be utilized. In some embodiments, the default value may be determined to be relatively high, since that may result in relatively small changes in a PIP correction value (Pcorr), thus providing a relatively conservative approach.

In some embodiments, during active mode 306, the controller delivers continuously adjusted ventilation to the patient, which may include closed loop control of one or more patient and/or ventilation related parameters.

In some embodiments, during active mode 306, a number of ventilation parameters are continuously adjusted, including FiO2 and PEEP. Other parameters that are adjusted during ventilation mode 308 in a continuous manner may include PIP, Vt, Ve, RR and I:E. In some embodiments, FiO2 is continuously adjusted based on a target patient oxygenation level, such as an SpO2 of 94%. In some embodiments, FiO2 starts at 21% (or, e.g., 20-25%). However, some embodiments, a user may select an initial FiO2 setting, such as between 21%-100%, for example.

On a continuous basis, central control 308 may provide data to ventilation control 310, including current EtCO2 as well as pressure (P) and volume (V) waveforms, and/or parameters derived at least in part from the P and V waveforms. Ventilation control 310 may use that data, potentially in addition to other data, in determining values for Vt, Ve, PIP, RR and I:E, which it then sends to central control 308. Central control 308 may then implement any appropriate adjustments to Vt, Ve, PIP, RR and I:E settings based at least in part on the sent values.

Furthermore, on a continuous basis, central control 308 may provide data to FiO2 control 312, including current patient SpO2. FiO2 control 312 may use that data, potentially in addition to other data, in determining a value for FiO2, which it then sends to central control 308. Central control 308 may then implement any appropriate adjustment to the FiO2 setting based at least in part on the sent values. Adjustment of the FiO2 setting may be accomplished via appropriate actuation of an oxygen source valve, or by adjusting an oxygen concentration from an oxygen supply, or in other ways.

Still further, on a continuous basis, central control 308 may provide data to PEEP control 314, including the current FIO2 and current PEEP. PEEP control 314 may then use that data, potentially in addition to other data, in determining an updated value for PEEP, which it then sends to central control 308. Central control 308 may then implement any appropriate adjustment to the PEEP setting based at least in part on the sent value. Adjustment of the PEEP setting may be accomplished via appropriate actuation of an exhalation valve of the respiratory distress ventilator 4102, for example.

It is to be understood that, while central control 308, ventilation control 310, FiO2 control 312 and PEEP control are described separately and communication with each other, in some embodiments, some or all of these aspects may be combined or integrated, or may function independently. In such embodiments, communication between combined or integrated aspects may be less or may be unnecessary.

Aspects of the present disclosure are include systems and methods that include generating context sensitive guidance (CSG) for medical diagnostics and the delivery of medical interventions by medical treatment and diagnostic devices, such as by a respiratory distress ventilator 4102 as depicted, for example, in FIG. 1. Such systems and methods may additionally provide a guided user interface for aspects of the context sensitive guidance directed at and interactive with a provider of medical care. The guided user interface be implemented at a companion device communicatively coupled via wired and/or wireless communication link(s) to one or more medical treatment and/or diagnostic devices, e.g., a patient monitor, a patient monitor/defibrillator, a ventilation system or device (such as a respiratory distress ventilator 4102) and/or a drug delivery device, used for medical assessments interventions during an emergency medical event, for example. In some embodiments, a companion device, or another device or system, on which CSG is implemented may be a remote control device or system for a respiratory distress ventilator 4102, for example.

The companion device (which, in some embodiments, may be a remote control device for the respiratory distress ventilator 4102) may be a mobile computing device such as a tablet, a personal display/digital assistant device, or a phone. In an implementation, the companion device(s) may be pre-configured to be associated with the medical treatment and/or diagnostic device so as to streamline wireless communication pairing without having to undergo a time consuming inquiry and response negotiation for a secure connection to be established. In some embodiments, the companion device configured to operate with a medical treatment device can be used in an emergency medical services (EMS) environment by trained emergency responders at the scene of an emergency or during prehospital transport. It can also be used in EMS during the ground and air transport of patients between medical facilities. The companion device, in some examples, can also be used in a hospital emergency room, general medical-surgical and intermediate care floors, cardiac care unit, electrophysiology (EP) lab, operating rooms, and other similar areas of the hospital and/or for intra-hospital transport of patients.

In an example scenario, an emergency responder or other medical caregiver tasked with treating a patient or victim of an emergency event, may first observationally evaluate the presentation of the patient. For example, such an evaluation likely includes observation of patient responsiveness, any respiratory distress, mental confusion, visible wounds. Additionally, the caregiver may converse with a conscious patient or a family member or friend of an unconscious patient to determine a chief complaint, health history, or other information relevant to treatment. In initiating care, the caregiver may start with an Airway-Breathing-Circulation (ABC) evaluation. Based at least on the ABC evaluation, the caregiver may connect the victim to a ventilation device, a patient monitor, and/or a patient monitor/defibrillator to treat a patient presenting with abnormal respiratory and/or hemodynamic conditions. The caregiver may further step through a OPQRST assessment for any pain described and/or exhibited by the patient. “OPQRST” is a mnemonic for emergency care providers that reminds them to evaluate Onset, Provocation/Palliation, Quality, Radiation, Severity, and Time associated with patient pain.

A patient presenting with respiratory distress generally requires a rapid and accurately targeted intervention. The caregiver may need to identify and implement the appropriate efficacious intervention within minutes in order to ensure that the patient stays alive and neurologically intact. Such an intervention includes actions that contribute directly to the care of the patient's presenting condition(s) and may determine the disposition of the patient. The intervention may or may not treat the etiology of the presenting condition(s). However, if the presenting condition(s) remain unchanged by an intervention, the patient may not live long enough for a treatment of the root cause.

For example, a patient may exhibit respiratory distress due to congestive heart failure (CHF). In this case, the respiratory distress has a cardiac etiology. An intervention to relieve the RD, such as providing oxygen to the patient to remedy hypoxemia, is necessary to keep the patient alive and neurologically intact until the cardiac etiology can be diagnosed and addressed with treatment. The treatment for the heart failure may be separate from the oxygen intervention. For example, this treatment may include angioplasty, high blood pressure medication, and/or arrhythmia medication.

As another example, the patient may exhibit respiratory distress due to pneumonia. In this case, the respiratory distress has a respiratory etiology. Here, an intervention to relieve the RD, such as providing oxygen, is necessary until the respiratory etiology can be diagnosed and addressed with a treatment. For example, the treatment may include administration of antibiotics for the pneumonia infection.

As a further example, a patient may be briefly exposed to an oxygen poor gaseous environment. For example, the patient may be in a car or home with a carbon monoxide leak and thus be exposed to low oxygen levels. In this case, an intervention of providing oxygen also treats the root cause of the RD. In this case, once the root cause of the patient's oxygen deficiency is diagnosed, the intervention will have functioned as a treatment.

Thus, as the above examples demonstrate, all treatments are interventions but not all interventions are treatments. A treatment includes actions by the caregiver that address both presenting conditions and the root cause, or etiology, of the presenting conditions. An intervention includes actions by the caregiver in response to particular presenting conditions. In some cases, such actions may only address presenting conditions and therefore may not be a treatment for the etiology of the conditions. In other cases, such actions may address presenting conditions along with the root cause of the conditions.

The context sensitive guidance (CSG) system, as described herein, provides care guidance (whether determined by and/or provided by a respiratory distress ventilator 4102 4102, a remote control device, or another device or system) in the context of initial presenting conditions of a patient before any reasonable accurate diagnosis is likely or in some cases before such diagnosis is even possible. The CSG system collects objective physiological and other medical data for the patient and identifies therapeutic interventions tailored to alleviate physiological conditions indicated by the data. The care guidance identifies and enables implementation of interventions directed at the initial presenting conditions which in some cases pose an acute and immediate risk to the patient's life. Once the patient is stabilized, the CSG system may provide further guidance in identifying a diagnosis, or specific root cause, and identifying and implementing treatments based on the diagnosis. Additionally, the CSG system may monitor the patient for indicators of a resumption of an acute and possibly life-threatening medical condition. In various embodiments, CSG may be determined and/or provided at devices including a respiratory distress ventilator 4102, device that remotely controls the respiratory distress ventilator 4102, and/or other devices or systems, whether local or offsite.

Based on an initial presentation of the patient and physiological data characterizing the initial presentation, a caregiver and/or medical device system implements a care plan that includes a set of interventions directed at the initial presentation. As care progresses, the caregiver and/or the medical device system accumulate data, for example, from physiological sensors, diagnostic tests and images, such as blood tests, x-rays, ultrasounds, etc., and observed or measured patient statuses before and after the various interventions administered to the patient. This data informs a differential diagnosis process. In the differential diagnosis process, etiologies of the impaired health are ruled out in a stepwise fashion with a resulting identification, or diagnosis, of one or more etiologies, or root causes. The goal of this process is a diagnosis, in other words, a definitive identification of one or more root causes of a patient's impaired health, and an identification of one or more treatments for the root cause based on the diagnosis. The CSG system protects the patient's health during the differential diagnosis process by identifying and implementing interventions necessitated by adverse medical conditions stemming from the root cause.

In some cases, a CSG system operational in a pre-hospital environment may identify both interventions and a diagnosis. In other cases, the CSG system may identify interventions but not the diagnosis. The diagnosis may not occur until after the patient is transported to a hospital and the results of more extensive tests and attempted treatments are analyzed. However, CSG enables the efficacious provision of critical care, without which, in many cases, a patient will not survive or remain neurologically intact long enough to reach a diagnosis stage.

Returning to the example of RD, the breathing portion of the ABC evaluation may indicate a breathing status ranging from a patient with no difficulty breathing, to a patient with slight to severe difficulty breathing, to a patient that is alive but not breathing on their own at all. For those patients breathing without difficulty, the caregiver will initially direct their attention to non-respiratory presenting conditions, such as trauma or sepsis.

For those patients with difficulty breathing or those not breathing at all, the caregiver will monitor for both proper respiratory and cardiac functions. Respiratory distress may stem from a respiratory etiology, a cardiac etiology, or a combination root cause. However, regardless of the etiology, respiratory function and cardiac function are interrelated and presenting conditions of respiratory distress warrant cardiac monitoring and vice versa. Thus, the CSG system, as applied to a respiratory presentation of a patient, executes a respiration/ventilation (R/V) algorithm based on respiration and ventilation indicators for a patient in parallel with an algorithm based on hemodynamic indicators. Therefore, the caregiver of a victim of respiratory distress will connect the patient to the medical devices referred to above, namely the ventilation system (such as a respiratory distress ventilator 4102 4102) and the patient monitor or patient monitor/defibrillator. These medical devices may monitor and measure respiratory health indicators, for example, respiratory gas exchange and/or lung mechanics, and cardiac health indicators, for example, blood pressure, heart rate, and an electrocardiogram (ECG). The respiratory gas exchange measurements may include, but are not limited to, pulse oximetry and capnography and the lung mechanics measurements may include, but are not limited to lung elastance and lung resistance.

A couple of examples illustrate the need for parallel operation of the R/V algorithm and the hemodynamic algorithm in implementing CSG for a presentation of RD. As a first example, a patient presenting with respiratory distress may be suffering from pneumonia. The patient requires interventions directed at the RD. However, pneumonia often raises a patient's heart rate. If this heart rate goes too high, the elevated heart rate may be an abnormal and life-threatening condition leaving the patient in immediate danger of death due to cardiac malfunction. Thus, while monitoring and implementing interventions for the patient's RD, the CSG system must monitor the hemodynamic indicators and implement cardiac interventions as needed.

As a second example, a patient presenting with respiratory distress may be suffering from CHF. The patient requires interventions directed at the RD. However, the cause of the CHF may be an arrhythmia that leaves the patient vulnerable to stroke. Therefore, as in the pneumonia example, while monitoring and implementing interventions for the patient's RD, the CSG system must monitor the hemodynamic indicators and implement cardiac interventions as needed.

In both of these examples, it is important to recognize that in pre-hospital care, for example in response to a 911 call, on a battlefield, or in another emergency treatment environment, the caregiver may, at least initially, only be aware of the presenting conditions and conditions revealed by the medical devices. Thus, underlying and perhaps chronic conditions may not be apparent or known by the caregiver. Thus, the CSG system for respiratory distress based on parallel monitoring of R/V and hemodynamics is crucial in treating respiratory distress presentations. The pneumonia infection or the chronic arrhythmia are likely initially unknown to the caregiver and it is only by monitoring both physiological systems that interventions may be properly applied to keep the patient alive long enough to reach a subsequent diagnosis of these etiologies. This stands in contrast to a non-emergency hospital setting or physician's office where the patient and the patient's health history are typically known and/or at least readily accessible to the caregiver.

The CSG system may reside on the companion device described above, on a medical device (such as, e.g., a respiratory distress ventilator 4102, or CCM/defibrillator), another computing device or system, on a cloud server, or a combination thereof. The CSG system may be communicatively coupled to the medical devices and receive inputs from the medical devices that include physiological data collected from the patient by the medical devices along with data describing actions taken by the medical devices during care of the patient. The CSG system may also provide a guided user interface and receive inputs from the caregiver at the guided user interface regarding patient medical data and/or actions taken by the caregiver. Based on this received data, the CSG system may identify clinical interventions and update previously identified interventions and may generate etiology estimates and determinations. The CSG system may then instruct and/or control medical devices to implement the clinical interventions and/or provide patient record data and event markers to the medical devices. The CSG system may also request specific patient data from the medical devices. The CSG system may access medical records for the patient (e.g., an electronic patient care record (ePCR), i.e., a patient chart, generated during an encounter, an archived patient chart, a record in a health information exchange (HIE) or other regional data base, and/or a hospital electronic health record (EHR). Further, the CSG system may provide prompts and/or instructions for the caregiver at the guided user interface order to implement the clinical interventions and guide patient care.

Referring to FIG. 30, an example of a device configuration for context sensitive guidance is shown. A quantity of each component in FIG. 30 is an example only and other quantities of each, or any, component could be used. As shown in FIG. 30, at patient care scene 100, a rescuer or caregiver 103 attends to a victim or patient 101 (the terms are used interchangeably here to indicate a person who is the subject of intended or actual medical treatment(s) and/or interventions), such as an individual who is in RD. The emergency care scene 100 can be, for instance, at the scene of an accident or health emergency, in an ambulance, in an emergency room or hospital, or another type of emergency situation. The rescuer 103 may be, for instance, a civilian responder with limited or no training in lifesaving techniques; a first responder, such as an emergency medical technician (EMT), a paramedic, a police officer, a firefighter; or a medical professional, such as a physician or nurse. The rescuer 103 may be acting alone or may be acting with assistance from one or more other rescuers, such as a partner EMT 104.

In this illustration, the rescuers 103, 104 may deploy medical/other device(s) 180 (such as, e.g., a respiratory distress ventilator 4102, CCM/defibrillator, other medical or monitoring device, or other portable computing device) coupled to patient interface devices 170. For example, the medical/other device(s) 180 may be medical therapy delivery device(s) configured to gather physiological data from the patient via the patient interface devices 170, monitor this data, and delivery therapy to the patient (e.g., electrotherapy or respiratory therapy) based on the gathered and monitored data.

The patient interface devices 170 may further include other sensors for monitoring parameters indicative of the patient's health status, e.g., physical parameters such as the patient's heart rate, temperature, respiration rate, blood oxygen level, blood glucose level, or other parameters indicative of the patient's health status. Some sensors, such as heart rate or ECG sensors, can be included in electrode pads coupled to a patient monitor/defibrillator. Additionally, one or more sensors may monitor treatment(s) and/or intervention(s) delivered to the patient 101. For instance, a compression puck can be positioned beneath the hands of rescuer 103 as the rescuer 103 administers CPR by detecting a rate, depth, or duration of compressions delivered to the patient 101. Additionally, airflow sensor included in, e.g., a respiratory distress ventilator 4102 may monitor volume and rate of ventilations administered to the patient 101 by rescuer 103, 104. Thus, some sensors can monitor both parameters indicative of the patient's health status and parameters indicative of the treatment and/or intervention delivered to the patient

The rescuers 103, 104 may use the at least one companion device 105. The companion device 105 may be a mobile device such as, for example but not limited to, a smartphone, a tablet, or a wearable device (e.g., watches or glasses). The rescuer(s) may also utilize additional computing devices, such as laptop computers or computing devices integrated into an ambulance, can be used to analyze health data about the patient or data indicative of treatments and/or interventions delivered to the patient or to communicate such data to a remote location (e.g., a dispatch center, an emergency room, or a remote server).

The companion device 105 and the medical/other devices 180 may communicatively couple to one another via a local wireless communication channel 199 established between the devices. In an implementation, the devices 105 and/or 180 may also communicatively couple to other medical and/or computing devices at the emergency scene 100. The communication channel 199 enables data to be securely and accurately shared between two or more devices at the emergency scene 100. During deployment of the medical/other device(s) 180 , these devices may store one or more patient encounter data files 188 that include records of data gathered during a patient case and of treatments and/or interventions by the medical device(s) along with records of device settings and operations during the patient case.

The companion device 105 may include a processor 108 and a memory 109. The memory 109 may store a CSG algorithm 121 that includes instructions executable by the processor 108 to implement a CSG processor module 120. The CSG algorithm 121 may implement a CSG user interface (UI) 110 at one or more input/output devices of the companion device 105 including, for example, a display and/or one or more audio and/or haptic devices. In other embodiments, however, one or more other devices, and the processors and memories thereof, may be used in these regards.

Additionally, the companion device 105 (or other device) may include a patient charting application 131. The patient charting application 131 may be generate an electronic patient care record (ePCR) as a contemporaneous record of caregiver observations, treatments and interventions provided, physiologic data for the patient, patient transport, transport destination, times and durations of patient care activities, emergency crew identification, patient demographic information, patient health insurance information, etc. The information entered into the ePCR may be entries from the caregiver captured by the companion device (e.g., touchscreen or keyboard entries, voice entries), entries via one or more input devices (e.g., scanner, microphone, camera, etc.), and/or automated entries from communicatively coupled devices or databases. The application 131 may operate in parallel or in cooperation with the CSG algorithm 121. In an implementation, the CSG processor module 120 may implement the patient charting application 131 and the CSG algorithm 121 through a combined user interface.

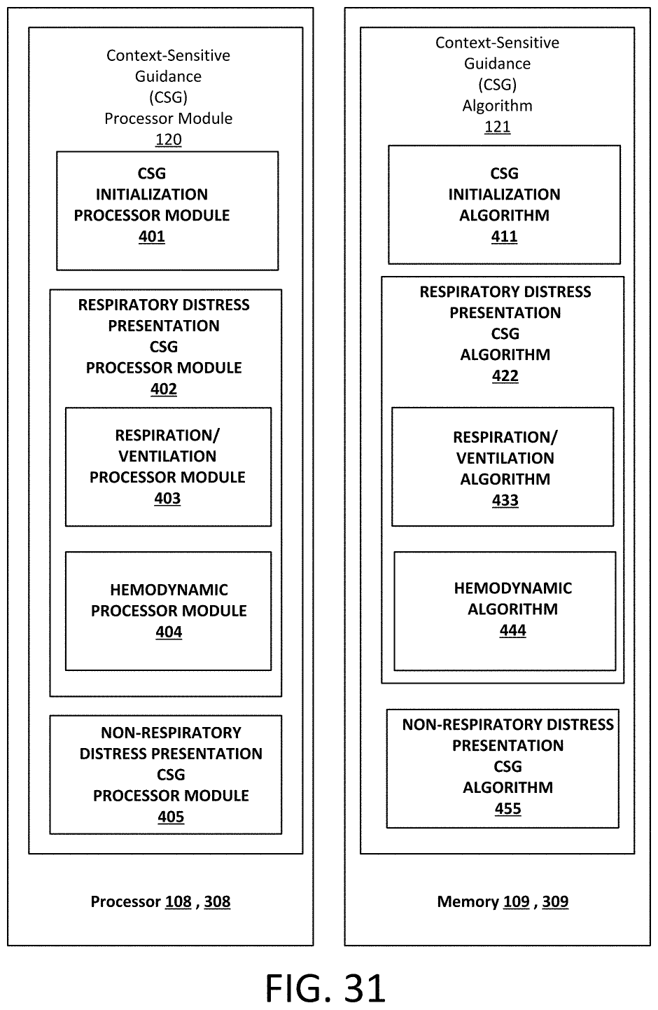

Referring to FIG. 31, examples of CSG algorithm and corresponding processor modules are shown schematically, such as may be stored and executed using a controller, including a processor and a memory, of a device (or system, local or offsite), such as a respiratory distress ventilator 4102, CCM/defibrillator, or other device or system, such as may be used in remote control of the respiratory distress ventilator 4102. The CSG algorithm 121 may include instructions stored in a non-transitory medium and executable by the device. The CSG algorithm 121, which may be stored in a memory of the device, includes a CSG initialization algorithm 411, a respiratory distress presentation CSG algorithm 422, and a non-RD presentation CSG algorithm 455. The CSG processor module 120, e.g., of the processor 108 at the companion device 105, may execute the CSG algorithm 121. The CSG processor module 120 includes a CSG initialization processor module 401, a respiratory distress presentation CSG processor module 402, and a non-RD presentation CSG processor module 405.

The example of the CSG algorithm 121 as described herein is divided based on a patient's presentation with or without RD. However, this is an example only and in other examples the CSG algorithm may be structurally organized around one or more other clinical conditions (e.g., patient presentation with or without cardiac distress, emergent vs. non emergent conditions, patient presentation with or without trauma, etc.). In practice, a patient often presents with multiple clinical conditions and therefore multiple algorithms run concurrently while managing the interactions with the user of the algorithms and communicatively coupled devices to guide, monitor, and control patient care.

The CSG initialization algorithm 411 is executable by the CSG initialization processor module 401. The CSG initialization algorithm 411 utilizes a caregiver assessment of a medical care recipient to initiate care and initiate the appropriate CSG algorithm based on the caregiver assessment.

In the presence of a respiratory distress presentation, the CSG initialization algorithm 411 initiates the respiratory distress presentation CSG algorithm 422, executable by the respiratory distress presentation CSG processor module 402. The respiratory distress presentation CSG algorithm 422 includes an R/V algorithm 433, executable by the R/V processor module 403, and a hemodynamic algorithm 444, executable by the hemodynamic processor module 404. The R/V algorithm 433 is discussed in more detail with regard to FIGS. 7-11 and the hemodynamic algorithm 444 is discussed in more detail with regard to FIG. 12. The R/V algorithm 433 guides, monitors, and/or controls patient respiration and ventilation care as provided by the medical device(s) 180 and the caregiver 103. The hemodynamic algorithm 444 guides, monitors, and/or controls patient hemodynamic care as provided by the medical device(s) 180 and the caregiver 103.

In the absence of a respiratory distress presentation, the CSG initialization algorithm 411 initiates the non-RD presentation CSG algorithm, executable by the non-RD presentation CSG processor module 405. In various implementations, the non-RD presentation CSG algorithm 455 may perform functions similar to those described herein for the respiratory distress presentation CSG algorithm 422. However, rather than performing these functions in the context of a respiratory presentation, the non-RD presentation CSG algorithm performs these functions in the context of a non-RD presentation such as, for example, but not limited to, cardiac presentation, trauma presentation, sepsis presentation, etc. In these cases, the medical/other device(s) 180 may include the ventilation system 280, the patient monitor/defibrillator 285, and/or other medical/other devices as appropriate for the particular patient presentation. In an implementation, the respiratory distress presentation algorithm 422 and the non-RD presentation algorithm 455 may run in parallel for a patient with multiple presentations.

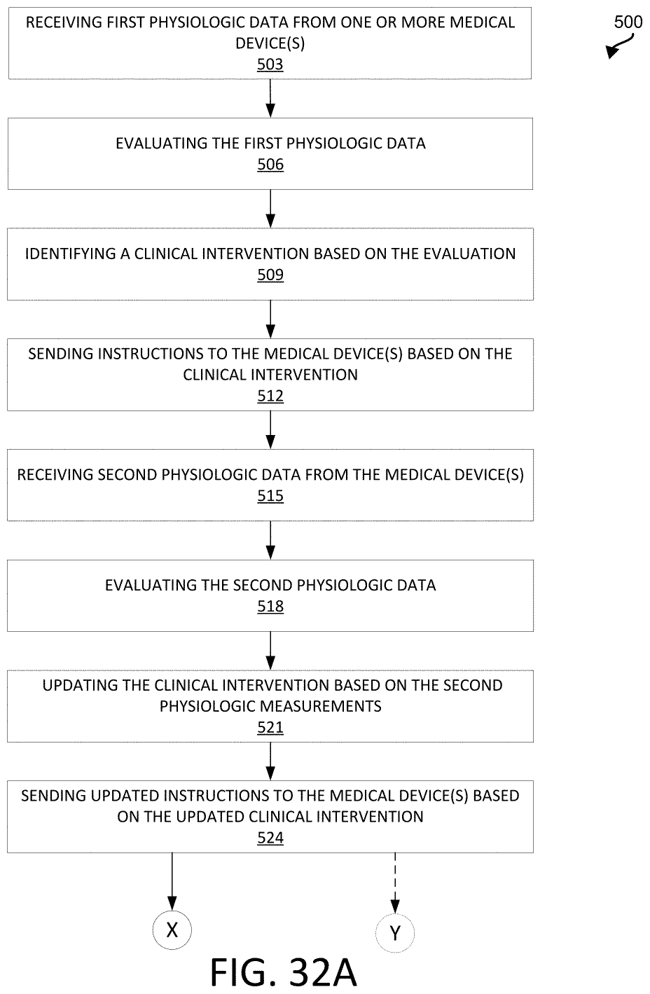

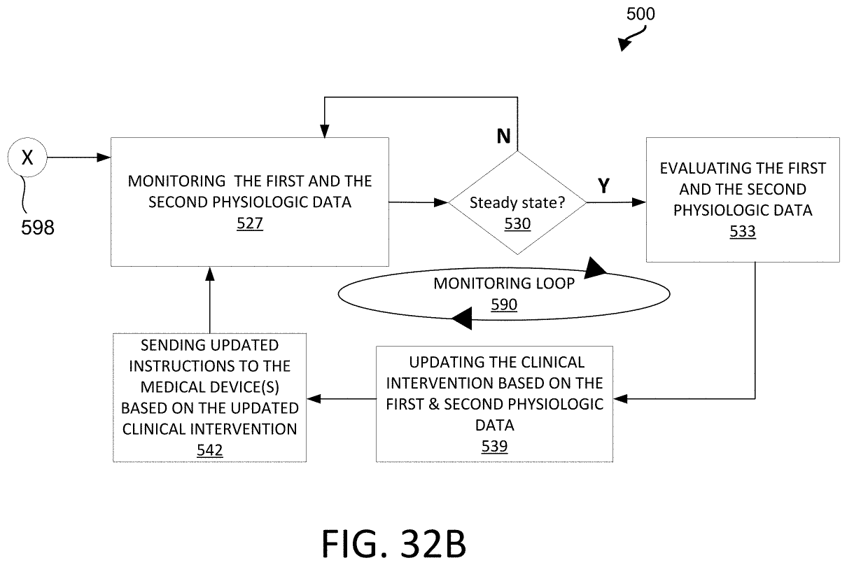

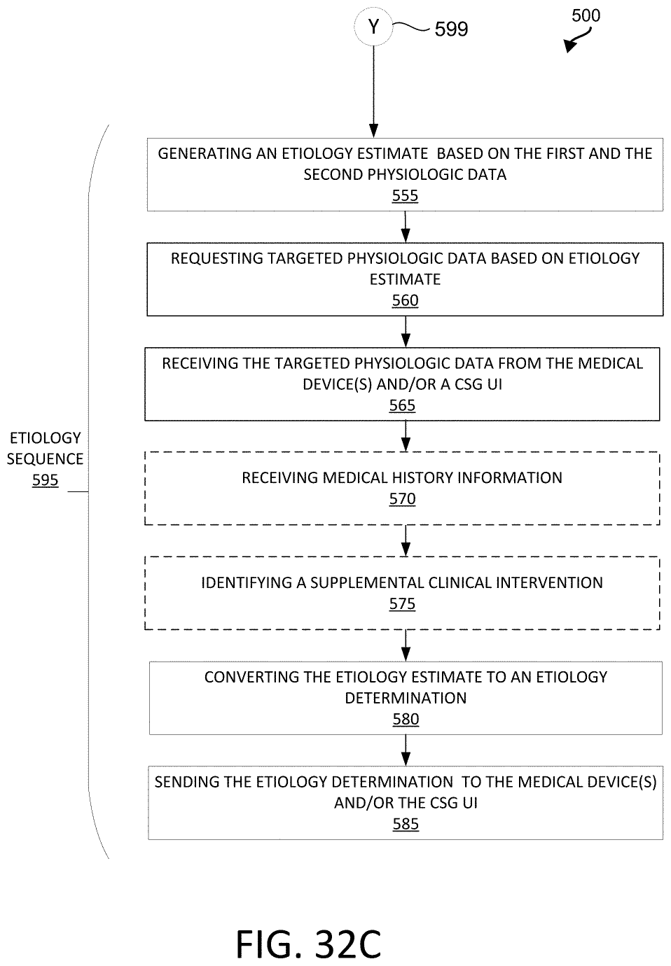

Referring to FIGS. 32A-C, an example of a method 500 of providing context sensitive guidance is shown. The method 500 is, however, an example only and not limiting. The method 500 can be altered, e.g., by having stages added, removed, rearranged, combined, and/or performed concurrently. In various other exemplary implementations, the CSG algorithm 121 may apply the method 500 to another patient presentation such as, for example, but not limited to, respiratory, cardiac presentation, trauma presentation, sepsis presentation, neurologic presentation (e.g. stroke, drug overdose) etc.

At the stage 503, the method includes receiving first physiologic data from one or more medical device(s). For example, the CSG processor module 120 at the companion device 105 or a cloud server may receive the first physiologic data from the medical/other device(s) 180. In an implementation, the caregiver 103 may couple the medical/other device(s) 180 to the patient or victim 101 via the patient interface devices 170 to initiate patient care. The patient interface devices 170 may collect and/or measure one or more physiologic parameters for the patient 101. The medical/other device(s) 240 may provide the first physiologic data as CSG input to the CSG processor module 120.

In an implementation, the stage 503 may also include receiving a portion of the first physiologic data from the CSG UI 110. The caregiver 103, 104 may provide user input to the user interface 110 that includes physiologic data such as, for example, a caregiver observation and/or patient information measured by the caregiver without use of the medical device(s) 180. The CSG UI 110 may provide the user input as CSG input to the CSG processor module 120.

At the stage 506, the method includes evaluating the first physiologic data. For example, the CSG processor module 120 at the companion device 105 or the cloud server(s) 398 may execute the CSG algorithm 121 to evaluate the first physiologic data. In an implementation, the CSG algorithm 121 may evaluate all or a portion of the first physiologic data and may focus the evaluation on one or more physiologic parameters that determine an initial intervention. The initial intervention may be a same intervention regardless of the etiology of the patient's presentation. For example, physiologic parameters that indicate abnormal oxygen levels, cardiac arrhythmia, or significant blood loss indicate initial life-saving interventions. In the case of abnormal oxygen levels, the etiology could be, for example, exposure to a gas leak or asthma. Although these are different etiologies that may require different treatments later in the care of the patient, the initial intervention of supplemental oxygen may be the same.

In various implementations, the CSG algorithm 121 may evaluate the first physiologic data according one of a variety of analyses. For example, evaluation of the first physiologic data may include comparison of one or more physiologic parameters to a target range, a target minimum value, and/or a target maximum value for the parameter. Each parameter may correspond to a respective target range, minimum value, and/or maximum value. The CSG algorithm 121 may flag any of the one or more parameters that are outside of a target range, below a minimum value, and/or above a maximum value.

At the stage 509, the method includes identifying a clinical intervention based on the evaluation. For example, the CSG processor module 120 at the companion device 105 or a cloud server may execute the CSG algorithm 121 to identify the clinical intervention.

The CSG algorithm 121 determines or selects at least one clinical intervention based at least in part on the received physiologic data and the evaluation of that data. In some implementations, the CSG algorithm 121 may receive and evaluate medical history data and/or data entered by the user at the CSG UI 110 based on caregiver observations and evaluations. The CSG algorithm 121 may select the at least one clinical intervention from a range of interventions according to a protocol (e.g., clinical practice guidelines and/or standards of care) that may include, for example, among others: pharmacologic, supplemental O2, mechanical respiratory or ventilation support (which could include various forms of CLC), additional monitoring, or logistical instructions relative to the management of the patient. The CSG algorithm 121 may include one or more pre-determined clinical interventions. Each clinical intervention may correspond to particular values of one or more physiologic parameters. The CSG algorithm 121 may access protocols (e.g., the clinical practice guidelines and/or standards of care) stored, for example, in one or more memories. These protocols may be pre-determined by a user of the CSG system and/or by an administrator or medical director of an emergency agency or other care provider service. In an implementation, the protocols may be customizable by the user, administrator, and/or the medical director. In an implementation, the CSG algorithm 121 may allow customization of the protocol at the time of care. In an implementation, a remote caregiver may select, provide, create, and/or customize or otherwise edit the pre-determined protocols.

At the stage 512, the method includes sending instructions to the medical device(s) based on the clinical intervention. The instructions may include implementation instructions for the clinical intervention and/or event marker(s) for the clinical intervention. For example, the CSG processor module 120 at the companion device 105 or a cloud server may send the instructions to the medical device(s) 180 as CSG output.

The implementation instructions may include control signals that cause the medical/other device(s) 180 to initiate or implement a particular intervention and/or to set intervention delivery or operational parameters for the medical/other device(s) 180. In an implementation, the medical/other device(s) 180 and/or the CSG UI 110 may request a user confirmation. The medical/other device(s) may initiate or implement a particular intervention and/or change or set delivery or operational parameters in response to receiving the user confirmation.

The instructions may include instructions for the medical/other device(s) 180 to record one or more event markers relevant to the evaluation of the first physiologic data and/or to the identified clinical intervention. In an implementation, the instructions may include the event marker. The medical/other device(s) 180 may record the one or more event markers in response to receiving the event markers and/or the event marker instructions from the CSG processor module 120. The medical/other device(s) 180 may record the one or more event markers in the patient encounter data file(s) 188 in the memory 187. In an implementation, the medical device(s) may transmit the patient encounter data file(s) 188 to a cloud server during or after the patient encounter for storage in a memory as medical device data files. In an implementation, the medical/other device(s) 180 may transmit the one or more event markers to a cloud server for recordation in the medical device data files.

In an implementation, the method may include sending instructions to the CSG UI 110 as CSG output 225. The instructions sent to the CSG UI 110 may include a prompt for the caregiver to perform a particular intervention or other caregiving activity, a prompt for the caregiver to provide a confirmation or other information prior to the intervention, a prompt for the caregiver to adjust or set particular parameters at the medical device(s) 180, a prompt for the caregiver to couple or de-couple a particular patient interface device 170 to or from the patient, an alarm for the caregiver, and/or a message that informs the caregiver of actions to be taken or already taken by the medical/other device(s) 180. The instructions sent to the CSG UI 110 may also include coaching or other instructional feedback to ensure proper performance of a caregiving task by the caregiver.

Sending instructions to the CSG UI 110 may include sending instructions to one or more companion devices. For example, in an implementation, the CSG processor module 120 may send instructions and/or other CSG output 225 to one or more companion devices 105 at the scene of the patient. As another example, in an implementation, the CSG processor module 120 may send instructions and/or other CSG output to one or more companion devices at the patient scene and to one or more companion devices located offsite from the patient scene. In an implementation, an offsite caregiver may view the CSG interface 110 at an offsite companion device. The offsite caregiver may provide user input to the CSG interface 110 at the offsite companion device and the offsite companion device 105b may transmit this user input to the a companion device, the medical/other device(s) 180, and/or a cloud server via one or more wired or wireless connections or networks.

At the stage 515, the method includes receiving second physiologic data from the medical/other device(s). For example, the CSG processor module 120 at the companion device 105 or a cloud server may receive the second physiologic data from the medical/other device(s) 180. The second physiologic data may be the same type of data as the first physiologic data, a different type of data, or a combination thereof.

For example, the first physiologic data may include a pulse oximetry (SpO2) measurement or waveform and an end-tidal carbon dioxide (EtCO2) measurement or waveform. The second physiologic data may include lung mechanics data (e.g., resistance and elastance), which is a different type of data than the first physiologic data. Alternatively, the second physiologic data may include updated measurements of the same type of data as the first physiologic data, for example, updated SpO2 and/or EtCO2 data. As a further alternative, the second physiologic data may include updated measurement of the first physiologic data combined with a different type of data. For example, the second physiologic data may include updated SpO2 and/or EtCO2 data combined with lung mechanics data (e.g., resistance and/or elastance data).

As another example, the first physiologic data may include blood pressure and heart rate data. The second physiologic data may include updated blood pressure and heart rate data. Alternatively, the second physiologic data may include a new type of data, for example, ECG data. As a further alternative, the second physiologic data may include a combination of updated blood pressure and/or updated heart rate data with the new type data, for example, the ECG data.

As discussed above, the instructions to the CSG UI 110 may include an instruction for the caregiver to couple or de-couple a particular patient interface device 170 to or from the patient. In an implementation, the patient interface device 170 that the caregiver couples to the patient in response to this instruction may be the source of the second physiologic data. For example, a caregiver may initially couple the patient to the patient monitor/defibrillator 285 as the source of the first physiologic data (e.g., the SpO2 and the EtCO2 data) and subsequently couple the patient to the ventilation system 280 as the source of the second physiologic data (e.g., the lung mechanics data).

In another example, the CSG UI 110 may include an instruction for the caregiver to couple an electroencephalograph (EEG) patient interface device 170 to the patient that is the source of the second physiologic data. In another example, the EEG may be configured to measure so-called depth of anesthesia via such methods as Bi-Spectral (BIS) index which may be used to distinguish between drug overdose and stroke. If the CSG engine determines that the more likely cause of the neurologic problem is drug overdose, the recommended therapeutic intervention will be delivering a naloxone dose.

At the stage 518, the method includes evaluating the second physiologic data. For example, the CSG processor module 120 at the companion device 105 or a cloud server may execute the CSG algorithm 121 to evaluate the second physiologic data. In various implementations, the CSG algorithm 121 may evaluate the second physiologic data according one of the variety of analyses described above with regard to the first physiologic data.

At the stage 521, the method includes updating the clinical intervention based on the second physiologic measurements. For example, the CSG processor module 120 at the companion device 105 or a cloud server may execute the CSG algorithm 121 to update the clinical intervention. The clinical intervention identified at the stage 509 may be a first clinical intervention and the updated clinical intervention from the stage 521 may be a second clinical intervention. Updating the clinical intervention may include maintaining the first clinical intervention in progress from the stage 512. In this case, the update is a validation of the ongoing clinical intervention. For example, if the first clinical intervention at the stage 512 was a high flow nasal cannula (HFNC) (which may be implemented by a respiratory distress ventilator 4102) at a particular flow rate and particular FiO2 (fraction of inspired oxygen), the update at the stage 521 may validate and continue such that the second intervention at the stage 521 is also HFNC at the same flow rate and FiO2. Alternatively, updating the clinical intervention may include continuing to provide the same type of clinical intervention but modifying the parameters of that clinical intervention. For example, if the first clinical intervention at the stage 512 was a high flow nasal cannula (HFNC) at a particular flow rate and particular FiO2 (fraction of inspired oxygen), the update at the stage 521 may modify the first interventions such that the second intervention at the stage 521 is also HFNC but at a different flow rate and/or FiO2. As a further alternative, updating the clinical intervention may include supplementing the first clinical intervention with the second intervention. For example, if the first clinical intervention at the stage 512 was a high flow nasal cannula (HFNC) at a particular flow rate and particular FiO2 (fraction of inspired oxygen), the update at the stage 521 may supplement the first intervention such that the second intervention is a combination of the first intervention (either maintained or modified) and one or more additional interventions. For example, the additional intervention may be CPAP provided with the HFNC or a pharmacological intervention provided with the HFNC or a combination of CPAP and a pharmacological intervention provided with the HFNC. As yet another alternative, updating the clinical intervention may include replacing the first clinical intervention with one or more second interventions. For example, at the stage 521 the system may terminate HFNC and replace with CPAP.

The CSG algorithm 121 may update the at least one clinical intervention based on a range of intervention types and parameters according to a protocol. The CSG algorithm 121 may update the at least one clinical intervention according to implementations substantially similar to those described above with regard to the stage 509 (e.g., the identification of the clinical intervention based on the first physiologic data).

At the stage 524, the method includes sending updated instructions to the medical/other device(s) based on and indicative of the updated clinical intervention. The updated instructions may include instructions for the medical/other device(s) 180 to implement the clinical intervention and/or instructions to record event marker(s) for the clinical intervention. For example, the CSG processor module 120 at the companion device 105 or a cloud server may send the updated instructions to the medical/other device(s) 180.

In an implementation, the stages 515, 518, and 521 may occur substantially concurrently or in rapid succession to the stages 503, 506 and 509. If the medical/other device and sensors providing the first and second physiologic data are the same, the first clinical intervention may occur as rapid response by the caregiver to the patient presentation in initiating the intervention and the second clinical intervention may fine tune that response based on data collection by the medical devices and sensors. However, if the caregiver has to connect the patient to initial sensors to receive the first physiologic data and then additional sensors to receive the second physiologic data, the temporal proximity of these stages may depend on the speed at which the caregiver can connect the various sensors. Furthermore, the first physiologic data is not limited to a single type of data but may be one or more types of physiologic data collected based on the caregiver's initial impression. For example, as discussed below, if a patient exhibits respiratory distress, the first physiologic data may include both SpO2 and EtCO2 data to evaluate the respiratory distress. The first physiologic data may also include heart rate, blood pressure, and other vital signs used to initially characterize or triage the patient condition. Similarly, the second physiologic data is not limited to a single type of data. This second physiologic data may be the same type or types of data as the first physiologic data but corresponding to a later time. Thus, the second physiologic data may reflect changes in the patient's condition in response to the first intervention according to the same metrics. Alternatively, the second physiologic data may add new types of data by the addition of sensors which may not only reflect changes in the patient's condition in response to the first intervention but may also refine the characterization of the patient's condition by adding additional metrics. For example, as discussed below, the first physiologic data may include the respiratory gas analysis (e.g., SpO2 and EtCO2) and then the second physiologic data may include lung mechanics data (e.g., lung elastance and resistance). The lung mechanics data may enable the caregiver and the medical devices to refine the intervention based on the more detailed understanding of the patient condition afforded by the addition of the second physiologic data.

At the stage 524, the method 500 follows the “X” connector to continue to the monitoring loop 590 shown in FIG. 32B. Additionally, the method 500 may optionally follow the “Y” connector to continue to the etiology sequence 595 shown in FIG. 32C. The monitoring loop 590 runs concurrently with the etiology sequence 595.

As shown in FIG. 32B, the method 500 enters the monitoring loop 590 at the X connection point 598. At this point in the method 500, the patient is connected to the medical devices via the patient interface devices necessary to provide the first and second physiologic data. For example, the patient may be connected to a patient monitor/defibrillator via a SpO2 sensor and an EtCO2 sensor for the first physiologic data. Furthermore, the patient may also be connected to a respiratory distress ventilator 4102 via an airway pressure sensor and the pneumotachometer for the second physiologic data. The patient monitor/defibrillator may also be connected to the patient via electrodes to monitor heart rate, an NIBP sensor to measure blood pressure, and/or one or more of the other patient interface devices 170 as determined by the patient presentation. Thus, at the stage 527, the CSG algorithm 121 is monitoring the data acquired by the medical/other device(s) 180 (e.g., the first physiologic data and the second physiologic data) from the patient via the patient interface devices 170 attached to the patient. Further, at the stage 527, the first and second physiologic data is substantially continuously provided to the CSG algorithm 121 (e.g., non-invasive blood pressure (NIBP) measurements are typically taken every 5 minutes, heart rate is typically recalculated every QRS and updated approximately every one to two seconds, heart rate is calculated over an average of 2-6 QRS intervals, pulse oximetry measurements are updated approximately every one to two seconds, capnography is updated upon every detected breath, etc.).

Once the patient is connected to the medical/other device(s) 180, the patient remains connected and the CSG algorithm 121 monitors the data, for example, according to the monitoring loop 590, until the patient's presenting condition is resolved and/or until the care of the patient changes hands (e.g., EMS arrives at a hospital and transfers the patient to the care of the hospital, the patient leaves the ICU, the patient is discharged from the hospital, a caregiver determines that an emergent condition is resolved and/or diagnosed, the patient dies, etc.).

The data monitoring at the stage 527 occurs after the implementation and update of the clinical intervention by the medical/other devices in response to instructions at the stages 512 and 524 in FIG. 5A. At least part of the purpose of the data monitoring at the stage 527 is to enable an evaluation at the stage 533 of the effect of previously applied interventions on the patient's physiologic systems. Additionally, the purpose of the data monitoring at the stage 527 is to enable an evaluation of a patient status at the stage 533 in the absence of any intervention changes or supplements to determine if a change or supplement is required to remedy a condition identified based on the evaluation.

In order to properly evaluate the monitored data at the stage 533, the monitoring stage 527 and the evaluating stage 533 are separated by a steady state inquiry 530. This steady state inquiry 530 accounts for possible lags between the application of an intervention and the manifestation of that intervention in the physiologic data. More specifically, if the caregivers and medical/other device(s) 180 apply the intervention to the patient at a time T₁, the manifestation this intervention in the monitored physiologic data may not be instantaneous and may appear at a time T₂ where (T₂−T₁) is approximately equal to the aforementioned lag, also referred to as a steady state delay. The duration of this steady state delay for any particular intervention and sensor data combination depends on a number of factors including, but not limited to, the type of intervention, the type of sensor used to collect data indicative of the effects of the intervention on the patient, the location of the sensor on the patient's body relative to the target location of the intervention, the type of physiological response to the intervention, the number and types of co-occurring physiological conditions and/or comorbidities in the patient that impact the efficacy of the intervention, the number and types of co-occurring interventions that impact the efficacy of the intervention, and the electronic connections between the sensor, medical device(s), and user interfaces.

For example, the CSG algorithm 121 may provide instructions for the clinical intervention of HFNC in response to a data evaluation of SpO2 indicative of hypoxia. The CSG algorithm 121 may monitor the SpO2 signal at the stage 527 and evaluate this signal at the stage 533. However, the impact of the HFNC on the SpO2 signal may be associated with a steady state delay between the time the HFNC is applied to the patient and the time the monitored Sp02 signal indicates the effects of the HFNC on the patient's lungs. This delay may be differ in duration between a finger oximetry probe and an earlobe oximetry probe. Further the delay may depend on other factors affecting the oxygen saturation of the blood and the blood circulation for the particular patient (e.g., anesthesia, ambulation, blood pressure, weight, cardiac conditions, medications, etc.). The CSG algorithm 121 may apply an empirically derived steady state delay corresponding to an average lag time or an average lag time plus a predetermined number of standard deviations between the stages 527 and 533. For example, the duration of the steady state delay may be a value between 10-40 seconds or in some cases a value between 30-300 seconds. The CSG system may apply this delay any time data is monitored and evaluated after the application of an intervention. Thus, at the stage 530, the CSG algorithm 121 continues to monitor the physiologic data and waits for the duration of the pre-determined steady state delay until proceeding to the evaluation stage 533. If the system did not apply this steady state delay, then the system may adversely affect patient care by unnecessarily and/or incorrectly changing an intervention before evaluating data accurately indicative of the effect of the intervention.

At the stage 533, the CSG algorithm 121 evaluates the monitored first and second physiologic data in a manner similar to that described at the stages 506 and 518 as described above. Based on this evaluation, the CSG algorithm 121 updates the clinical intervention at the stage 539. As similarly described for the stage 521, updating the clinical intervention may include maintaining an ongoing intervention, modifying parameters of the ongoing intervention, supplementing the ongoing intervention, or replacing the ongoing intervention with one or more other and different interventions.

At the stage 536, the CSG algorithm 121 provides updated instructions to the medical device(s) based on the updated clinical intervention as similarly described for the stage 524. The CSG algorithm 121 then returns to the stage 527 to continue monitoring the physiologic data.

Optionally, in parallel with the monitoring loop 590, the method 500 may implement the etiology sequence 595 after the stage 524 and as shown in FIG. 5C. The method 500 may enter this sequence at the “Y” connection point 599 and proceed to the stage 555.

At the stage 555, the method includes generating an etiology estimate based on the first and the second physiologic data. For example, the CSG processor module 120 at the companion device 105 or a cloud server may execute the CSG algorithm 121 to generate the etiology estimate. Based on the first and second physiologic data, the CSG algorithm 121 may initiate an etiology distinction engine with an etiology estimate. The etiology estimate indicates a broad etiology category clinically indicated by the first and second physiologic data and from which the etiology distinction engine may convert the etiology estimate to an etiology determination. For example, the first and second physiologic data for respiratory distress may indicate an etiology estimate of an obstructive lung condition. However, the obstructive lung condition is an estimate rather than a determination because the obstructive lung condition may originate from a variety of different underlying conditions such as, for example, chronic obstructive pulmonary disease (COPD) and asthma. In order to distinguish between these two etiologies, the CSG algorithm 121 may require additional information beyond the first and second physiologic data. However, the first and second physiologic data enable the CSG algorithm 121 to guide, monitor, and control interventions that provide therapy, potentially life-saving therapy, to the patient while the diagnostic process is ongoing. The benefit of the CSG algorithm 121 is that the interventions provided are context sensitive as they are tailored to the particular physiological context defined by at least the first and second physiologic data and the interaction between the CSG algorithm 121 and the medical/other device(s) 180.

At the stage 560, the method includes requesting targeted physiologic data based on the etiology estimate. For example, the CSG processor module 120 at the companion device 105 or a cloud server may request the targeted physiologic data from the medical/other device(s) 180. The CSG algorithm 121 may identify and the CSG processor module 120 may request one or more items of physiologic data that may differentiate between various etiology determinations within the scope of the broader etiology estimate. For example, an etiology estimate of an obstructive lung condition is indicative of both COPD and asthma. However, spirometry data and/or exhaled nitric oxide data may differentiate between COPD and asthma. These one or more items of physiologic data may individually differentiate between etiologies and/or may differentiate in the aggregate.

The CSG processor module 120 may send the request for the identified targeted physiologic data to the medical/other device 180 as CSG output and/or may send the request to the companion device 105 as CSG output to the CSG UI 110.

The request to the medical device(s) 180 may include an instruction to send the data or may include an instruction to collect and send the data. In an implementation, the medical/other device(s) 180 may collect the targeted physiologic data in response to the request from the CSG processor module 120. Thus the request may trigger a collection the targeted physiologic data by the medical/other device(s) 180. Alternatively, the medical/other device(s) 180 may extract the targeted physiologic data from the patient encounter data file 188 stored at the medical/other device(s) 180. The extracted and stored data may have been collected by the medical/other device(s) 180 prior to the request from the CSG processor module 120.

The request to the companion device 105 may generate a prompt at the CSG UI 110 for the caregiver to enter or to collect and enter the targeted physiologic data. Thus the caregiver may change a setting on the medical/other device(s) 180 and/or the patient interface devices 170 already connected to the patient to collect this data. Alternatively, the caregiver may couple an additional medical/other device 180 or patient interface device 170 to the patient to collect the requested data. The CSG UI 110 may provide a user entry field to capture the requested data in response to the user entry of this data.

At the stage 565, the method includes receiving the targeted physiologic data from the medical/other device(s) 180 and/or from the companion device 105. For example, the CSG processor module 120 at the companion device 105 or a cloud server may receive the targeted physiologic data from the medical/other device(s) 180 and/or from the CSG UI 110 at the companion device 105. The medical/other device(s) 180 and/or the companion device 105 may provide the targeted physiologic data automatically in response to the request from the CSG processor module 120 or may request a user confirmation prior to providing the targeted physiologic data.

Optionally, at the stage 570, the method may include receiving medical history information for the patient. Additionally, in some implementations, the method 500 may include receiving medical history information at one or more of the stages 502 and 515. Accordingly, one or more of the stages 509 and 521 may include identifying and/or updating clinical interventions based on the medical history. Similarly, one or more of the stages 555 and/or 580 may include generating and/or converting the etiology estimate based on the medical history. For example, the medical history for the patient may include a chronic condition, such as asthma, COPD, heart disease, or diabetes. Such conditions inform the clinical interventions and may simplify the conversion of the etiology estimate to an etiology determination. As another example, the medical history may include medications or diseases, such as communicable blood borne diseases, that may also inform the clinical interventions and/or simplify the conversion of the etiology estimate to the etiology determination. In both of these examples, the medical history may provide information that contraindicates one or more clinical interventions or that indicates a need or potential need for additional clinical interventions beyond those indicated by the physiologic information gathered by the medical/other device(s).

For example, the CSG UI 110 (such as on a respiratory distress ventilator 4102, tablet, CCM/defibrillator, or other device or system) may capture user input medical history for the patient. The CSG UI 110 may prompt the user and/or provide selectable input fields for the user to provide medical history. The user may obtain medical history from the patient and/or from an associate of the patient. For example, the user may interview the patient and/or the patient's associate to obtain medical history. The user or caregiver may also determine medical history by observation and/or evaluation of the patient's condition. For example, the user or caregiver may obtain information from a medic alert bracelet, from medication containers in the vicinity of the patient, from the scene of the patient's emergency (e.g., a chemical spill, a car accident, illegal drug paraphernalia, medical therapy equipment such as an oxygen tank or a wearable defibrillator, etc.). The user or caregiver may also observe symptoms of a chronic or historic condition by physical examination of the patient. The companion device 105 may provide information provided at the CSG UI 110 as CSG input 230 from the UI to the CSG processor module 120. The companion device providing the information may be on scene with the patient (e.g., the companion device 105a) and/or may be an offsite companion device 105b at an offsite location. An offsite caregiver may have knowledge of and/or access to patient records and may provide medical history as input to the CSG UI 110 at an offsite companion device.

As another example, the CSG processor module 120 may receive medical history information from one or more medical records databases via one or more networks. The CSG processor module 120 may send a query for the medical history information to one or more medical records databases. The CSG algorithm 121 may generate the query automatically during the process 500 and/or the CSG algorithm 121 may provide a user prompt and/or request user confirmation at the CSG UI 110 and generate the query in response to the prompt and/or the confirmation.

Optionally, in an implementation, the method 500 may include identifying one or more supplemental clinical interventions at the stage 575 based on the etiology determination and/or the medical history information. The CSG algorithm 121 may identify one or more clinical interventions, for example pharmacological interventions, specific to the etiology determination. For example, based on an etiology determination of COPD, the CSG algorithm 121 may identify administration of a bronchodilator as an additional clinical intervention whereas based on an etiology determination of asthma, the CSG algorithm 121 may identify administration of a corticosteroid as an additional clinical intervention.

At the stage 580, the method includes converting the etiology estimate to an etiology determination based on the targeted physiologic data and/or the medical history information. For example, the CSG processor module 120 at the companion device 105 or a cloud server may execute the CSG algorithm 121 to convert the etiology estimate to the etiology determination. As discussed above, the targeted physiologic data may differentiate between various etiology determinations within the scope of the broader etiology estimate. Thus, in continuation of the example provided above with regard to the stage 560, spirometry data showing a reduced forced vital capacity (FVC) may indicate COPD whereas spirometry data showing nearly normal FVC may indicate asthma. In this manner, the spirometry data may differentiate between two possible etiologies for the obstructive lung condition indicated by the first and second physiologic data. As another example, the swelling of lung tissue associated with asthma may increase a level of exhaled nitric oxide. Thus, this increased level may indicate an etiology determination of asthma on its own or, because such increases may also occur with COPD, such an increase in aggregate with spirometry data showing near normal FVC may support the etiology determination of asthma. In an implementation, a combination of targeted data may enable the conversion of an etiology estimate to an etiology determination. For example, the targeted data may include a combination of spirometry data, s3-s4 heart sounds, and lung fluid retention data as measured by RF-based lung fluid measurement. Together, these data enable a distinction between CHF and COPD.

In implementation, the CSG algorithm may utilize medical history data along with targeted data to convert an etiology estimate to an etiology determination. For example, spirometry data alone may not distinguish between various etiologies for an obstructive lung disease but spirometry along with medical history may provide this distinction. As more specific examples, spirometry combined with a medical history indicating exposure to asbestos may enable a etiology determination of asbestosis or spirometry combined with a medical history indicating coal dust exposure may enable an etiology determination of black lung disease. As another example, a medical history indicating low blood pressure and cardiac disease combined with an etiology estimate inclusive of CHF may enable an etiology determination narrowed to CHF.

At the stage 585, the method includes sending the etiology determination to the medical device(s) and a CSG UI. For example, the companion device 105 or a cloud server(s) may send the etiology determination to the medical device(s) 180. The companion device 105 may provide the etiology determination at the CSG UI 110 in response to an instruction from the CSG algorithm 121. In an implementation, a cloud server(s) may execute the CSG algorithm 121 to service a CSG application at the companion device 105 and provide the etiology determination at the CSG UI 110 at the companion device 105.

In an implementation, sending the etiology determination to the medical device(s) may include an instruction to record the etiology determination in the medical device data file for the patient encounter and/or may include an instruction to change and/or initiate a clinical intervention based on the etiology determination. For example, if the CSG algorithm 121 identifies a pharmacological intervention, the instruction may identify the medication along with a dose and administration instructions.

In an implementation, sending the etiology determination to the CSG UI 110 may include a prompt for a user confirmation of the etiology determination and/or an instruction to change and/or initiate a clinical intervention based on the etiology determination.

In an implementation, the CSG algorithm may also invoke non-linear evaluation methods at various stages of the method 500 described below (e.g., at the stages 506, 521, 533, and 536 to evaluate physiologic parameters and update the clinical intervention, at the stage 555 to evaluate physiologic data to generate an etiology estimate, at the stage 565 to identify a supplemental clinical intervention based on targeted data and, optionally, medical history). The non-linear evaluation of multiple physiologic parameters may function as a patient condition estimator where the estimated patient condition corresponds to a particular intervention. For example, an estimated patient condition of hypoxia corresponds to an intervention of delivering supplemental oxygen to the patient.

One example of a patient condition estimator is a Kalman filter. Generally, the Kalman filter models a linear system with Gaussian distribution, which may not be encountered in the physiologic setting. Thus at least one example of the patient condition estimator executes an extended Kalman filter (EKF) to solve the problem of non-Gaussian, nonlinear filtering. The EKF is based upon the principle of linearizing the measurements and evolution models using Taylor series expansions. The series approximations in the EKF process may, however, lead to poor representations of the nonlinear functions and probability distributions of interest. As a result, the EKF may diverge. Therefore, at least one example of the patient condition estimator executes an unscented Kalman filter (UKF) based on the hypothesis that it is easier to approximate a Gaussian distribution than it is to approximate arbitrary nonlinear functions. It has been shown that the UKF leads to more accurate results than the EKF and that it generates much better estimates of the covariance of the states (the EKF often seems to underestimate this quantity). The UKF, however, has a limitation in that it does not apply to general non-Gaussian distributions, as is often the case with ECG spectral distributions.

Another example of the patient condition estimator is a Sequential Monte Carlo process, also known as a particle filter. This example overcomes the non-Gaussian limitations and allows for a complete representation of the posterior distribution of the states, so that any statistical estimates, such as the mean, modes, kurtosis and variance, may be easily computed. Particle filters may, therefore, deal with any nonlinearities or distributions. Particle filters rely on importance sampling and, as a result, require the design of proposal distributions that can approximate the posterior distribution reasonably well. In general, it is hard to design such proposals. The most common strategy is to sample from the probabilistic model of the state evolution (transition prior). This strategy, however, may fail if the new measurements appear in the tail of the prior or if the likelihood is too peaked in comparison to the prior.

Some other example patient condition estimators include an estimator/predictor trajectory tracking technique known as the Unscented Particle Filter (UPF) and other non-linear estimators such as, for example, non-linear regression, neural networks, and convolutional neural networks.

In some embodiments, the CSG algorithm may be based, at least in part, on a Hidden Markov Model (HMM) with the sequence of patient condition and therapeutic intervention vectors as the input. A state transition matrix can be developed using HMI techniques known to those skilled in the art. In particular, the sequence of underlying patient states and recommended therapeutic interventions primitives, and condition and resultant intervention sentences is modeled as a hidden Markov model (HMM), defined as a variant of a finite state machine having a set of states, Q, an output alphabet, O, transition probabilities, A, output probabilities, B, and initial state probabilities, Π. The current state is not observable. Instead, each state produces an output with a certain probability (B). Usually the states, Q, and outputs, O, are understood, so an HMM is said to be a triple, λ=(A, B, Π). Each value of output alphabet, O, can be given a unique threshold and coefficient set. For instance, the HMM will have the transition probabilities stored in it, as a result either of training against a database or patient-specific training, indicating such understood “facts” (i.e. therapeutic intervention grammar). For instance, that it is a very low probability that a patient's blood pressure will decrease by more than 20 mmHg if they were just given a dose of a vasopressor: there must been either an intervening the blood pressure reading may be lost in noise, or, alternatively, blood pressure may decrease with sepsis as a more likely estimate of the patient's underlying condition, in which case the optimal therapeutic intervention would be to continue vasopressor but also give broad-spectrum antibiotic for potential septic infection, and perform point of care blood analysis to do a measure of lactic acid.

Other techniques known in the field of HMM classification may be employed. For instance, discriminative training techniques that dispense with a purely statistical approach to HMM parameter estimation and instead optimize some classification-related measure of the training data. Some examples include maximum mutual information (MMI), minimum classification error (MCE) and minimum phone error (MPE) and use of the Viterbi algorithm to find the best path. Other techniques are to keep a set of good candidates instead of just keeping the best candidate, and to use a better scoring function (re scoring) to rate these good candidates. The set of candidates can be kept either as a list (the N-best list approach) or as a subset of the models (a lattice). Re-scoring may be done to minimize the Bayesian risk.

In some embodiments, so-called Deep Learning Networks may be employed. Deep networks may be employed for unsupervised or generative learning to capture high-order correlation of observed or visible data for pattern analysis or synthesis purposes when no information about target class labels is available. Additionally, deep networks may be used for supervised learning to directly provide discriminative power for pattern classification purposes, often by characterizing the posterior distributions of classes conditioned on the visible data. Further, hybrid deep networks may be employed where the goal is discrimination which is assisted with the outcomes of generative or unsupervised deep networks. Other classification methods include, for example, Deep Neural Networks (DNN) and Recurrent Neural Networks (RNN), Convolutional Neural Networks (CNN), Restricted Boltzman Machine (RBM), Deep Belief Network (DBN), Deep Boltzman Machine (DBM), etc.

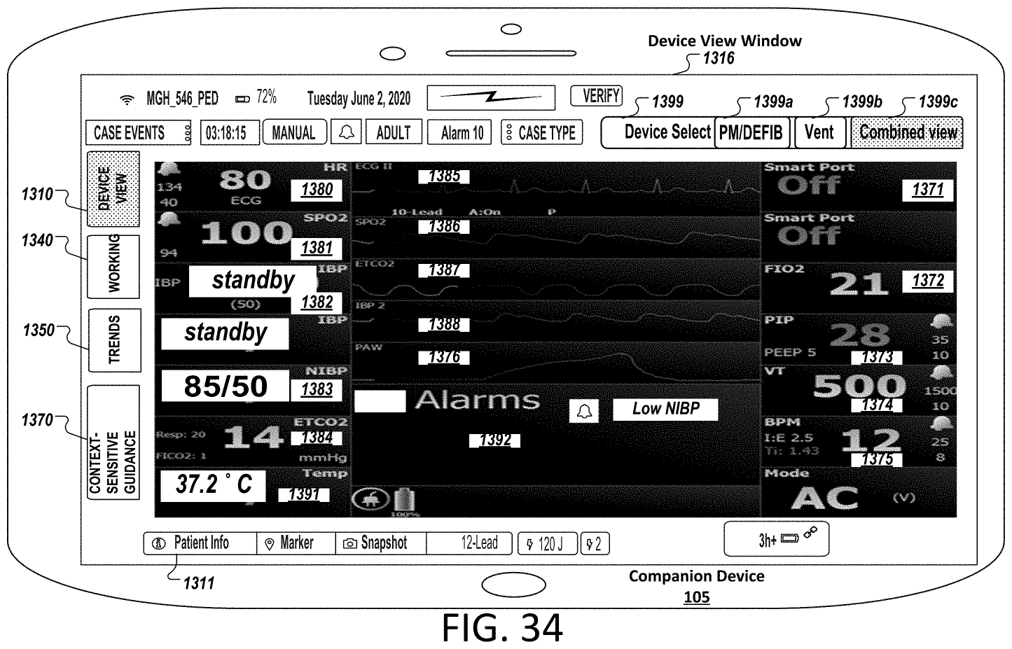

Referring to FIG. 33, a companion device (which may, in some embodiments, be a remote control device for remote control of a respiratory distress ventilator 4102) configured to provide a device view user interface is illustrated. The companion device 105 may be configured to provide a device view window 1315 as shown, for example, in FIG. 33. Another example of a device view window 1316 is shown in FIG. 34 as discussed below.

The device view may improve care from a team of caregivers and/or in a chaotic emergency environment where the screen of the medical device may not be readily accessible to all team members and/or may be inconveniently located for optimum viewing (e.g., under a gurney, on a moving gurney, under an ambulance bench, etc.). For example, a first caregiver 103 may attend to the victim 101 with assistance and therapy from the medical device, such as a patient monitor/defibrillator, and the medical device display screen 310. A second caregiver 104 may prepare medications, or a gurney, or attend to another task without a view of a medical device/other display screen. In some scenarios, the second caregiver 104 may supervise a larger care team and possibly other victims. The second caregiver 104 may view the device view window 1315 on the companion tablet 105.

In some implementations, the companion device can display sensor data in real-time from one or more physiological sensors connected to the medical treatment device. In some examples, the companion device can display a visual reproduction of the information displayed at the medical treatment device in a first display. This first display view, referred to as a device view, allows additional medical team members to participate in patient treatment without having to be immediately proximate to the medical treatment device. For example, a rescue team supervisor (e.g., the caregiver 104 in FIG. 33) who oversees and coordinates patient treatment during a critical patient care event (e.g., RD, chest pain, cardiac arrest, traumatic brain injury (TBI), RD, or critical care monitoring) can view, in real-time, the same waveforms and patient data displayed at the medical treatment device. This allows supervisors or other medical team members to observe both rescuer treatment actions and patient data simultaneously so that the supervisors can provide recommendations to further enhance patient treatment and coordinate treatment by multiple rescuers. In some examples, the device view displays a visual reproduction of case information displayed at the medical treatment device.

The medical device data displayed in the device view of the companion device 105 is a visual reproduction of that information displayed at a display interface of the medical/other device(s) 180 when one or more of the following visual aspects of the display interface of the medical/other device(s) 180 is reproduced in the device view: the display layout, magnification of each data section, physiologic waveform selection, physiologic numeric readout selection, resolution, waveform duration, waveform size, text size, font, and display colors. In some examples, the visual reproduction of the display screen of the medical/other device(s) 180 at the companion device 105, which may include respiratory aspects, can exactly replicate at is displayed at the medical/other device(s) 180 or one or more of the visual aspects may be altered from what is displayed at the medical device(s) 180. In one example, font and size of one or more items of displayed case information may be enlarged and/or highlighted to draw the eyes of users of the companion device 105 to the respective case information. Waveforms or other data of a certain type may be magnified or color coded in a particular fashion. For example, ECG waveforms, such as in a 12-lead analysis, may be magnified similarly, or certain waveforms may be emphasized in resolution, color, or other manner of reproduction. In some embodiments, numerical values representing non-continuous physiological measurements (e.g., NIBP, SpO2, HR, EtCO2) may be shown in a similar sized font or layout. In some cases, for example, a visual reproduction may be a replication of the information as presented on the display interface of the medical treatment device, or alternatively with slight variations thereof.

In some examples, the device view UI screen 1315 includes one or more selectable inputs at the display interface that cause more, different, or additional information to be displayed, cause one or more actions to be taken at the medical/other device(s) 180, or provide additional user input interface screens that allow users to submit information that can be transmitted to the medical/other device(s) 180. For example, the device view 1315 can include one or more view selection tabs 1310, 1340, 1350, and 1370 that allow the user to toggle between different display views for the display at the companion device 105. In one example, user selection of selection tab 1310 causes the device view UI screen 1315 to be displayed at the companion device 105. The device view UI screen 1315 can display a number of waveforms and metrics indicative of a condition of a patient and/or a status of medical treatment being administered to the patient. For example, as shown in FIG. 33, the device view window 1315 may display ECG waveforms 1320a and 1320b, a pulse oximetry waveform 1320c, and a capnography waveform 1320d along with temperature 1320e, invasive blood pressures 1320f and 1320g, heart rate 1325a, pulse oximetry (SpO2) discrete measurement 1325b, end-tidal carbon dioxide (EtCO2) discrete measurement 1325c, and non-invasive blood pressure (NIBP) 1325d.

In some examples, the visual reproduction may encompass an exact replication of the data displayed at the medical treatment device. In other examples, the visual reproduction may include data and formatting variations that can enhance viewing and comprehension of the case information by the companion device user. In some examples, display layout, magnification of each data section, physiologic waveform selection, physiologic numeric readout selection, resolution, waveform duration, waveform size, text size, font, and/or display colors may vary from what is displayed at the medical treatment device(s).

In some implementations, the information displayed at the device view UI screen 1315 may vary from the information displayed at a display interface of the medical device(s) 180. In some examples, the differences between the interfaces can include differences in a type of information displayed, a display layout, or a display format. For example, an amount of magnification of each data section, resolution, size, and screen position may vary between the device view UI screen 1315 and the display interface of the medical device(s) 180. Additionally, relative waveform sizes and colors, fonts, and text size may vary between the device view 1315 at the companion device 105. In one example, the device view UI screen 1315 may display numeric values of all physiological case information in a maximized, large-number format without waveforms. In some examples, one or more items of case information displayed at the device view UI screen 1315 may vary from the case information displayed at the display of the medical device(s) 180.

In an implementation, the device view window 1315 may include a patient information control 1311. Activation of the control 1311 may enable the user to enter patient identification information at the companion device 105.

Referring to FIG. 34, an example of a combined ventilation system/patient monitor-defibrillator device view user interface at the companion device is shown. In an implementation, one or more of the device view window 1315 and/or 1316, the working view window 1415, and the trend view window 1515 may include a device selection control 1399 (e.g., patient monitor/defibrillator selection control 1399a, ventilation system selection control 1399b, and combined device selection control 1399c). The control 1399 may enable the user to select data for display from a single medical/other device (e.g., the patient monitor/defibrillator 285 or the ventilation system 280, which may be a portable defibrillator or respiratory distress ventilator 4102) or select a combined display. In one example, case information for each type of medical treatment device is viewable at a respective separate display interface of the companion device. In another example, case information for different medical treatment devices can be combined within the same display interface screen at the companion device 105. In this way, the companion device user may customize the case information displayed at the companion device 105 as received from one or more of the medical/other device(s) 180.

The data provided in the example of the combined device view window shown in FIG. 34 includes data gathered by the ventilation system 280 by the patient monitor/defibrillator 285. For example, ventilation system 280 may provide the port information 1371, the fraction of inspired oxygen (FIO2) data 1372, the peak inspiratory pressure (PIP) 1373, the tidal volume (VT) 1374, and the breaths per minute (BPM) 1375. Additionally, the ventilation system 280 may provide the airway pressure waveform (PAW) 1376. A device view window dedicated to data gathered by the patient monitor/defibrillator 285 may not include these values. The patient monitor/defibrillator 285 may provide the HR 1380, the SpO2 value 1381, the invasive blood pressure (IBP) 1382, the NIBP 1383, the EtCO2 1384, the ECG waveform 1385, the SpO2 waveform 1386, the EtCO2 waveform 1387, and the IBP waveform 1376.

The patient monitor/defibrillator 285 or the ventilation system 280 may provide the temperature data 1391. One or more of the patient monitor/defibrillator 285 and the ventilation system 280 may provide alarms 1392.

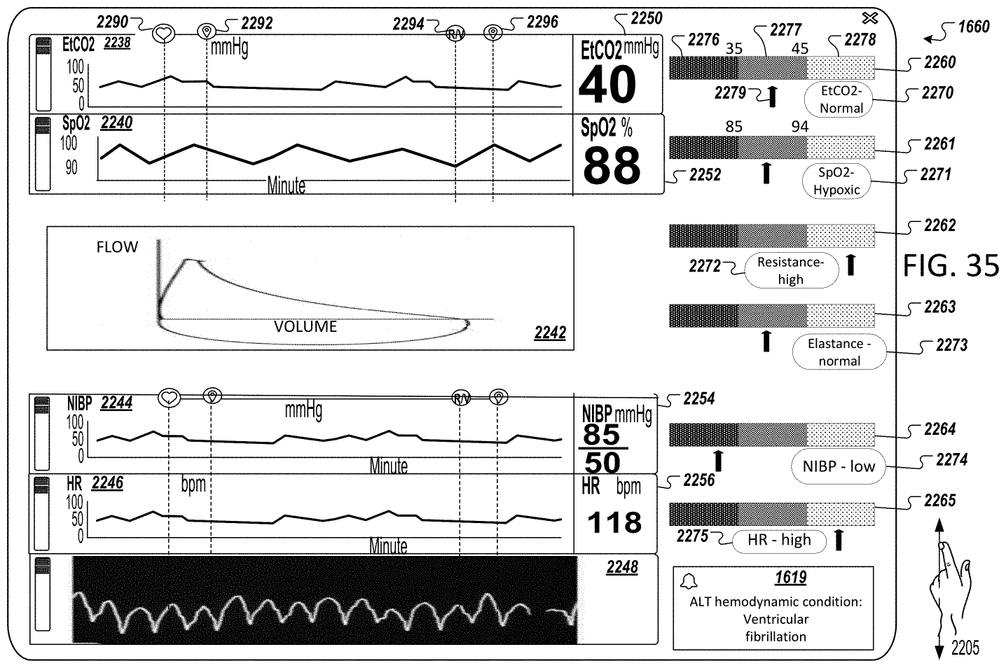

Referring to FIG. 35, an example of a medical device data screen is shown (which may be presented via the display of, e.g., a respiratory distress ventilator 4102, CCM/defibrillator, tablet, or other device or system, which may include a device capable of remote control of the respiratory distress ventilator 4102, for example). The medical device data screen 1660 may be a scrollable screen. The medical device data screen 1660 may be a scrolling window so that users can access a larger set of data with a scrolling gesture. In some implementations, the companion device 105 may allow the user to customize the data displayed at the medical device data screen 1660. For example, upon selecting a medical device data tab, the companion device 105 may display a list of available medical device data for the user to select and/or de-select based upon viewing preferences. In some cases, the data screen 1660 may be created and customized ad hoc during medical treatment by controls that allow the viewer of the companion device 105 to format the medical device data as they wish. In an implementation, the CSG algorithm 121 may pre-customize the data screen 1660 based on preferences or treatment roles of a user of the companion device

In an implementation, the medical device data screen 1660 may display physiologic sensor data received from the medical/other device(s) 180. The medical device data screen 1660 may display this data as trends and/or waveforms (e.g., EtCO2 2238, SpO2 2240, airway data 2242, NIBP 2244, heart rate 2246, and ECG 2248) over time during the patient encounter. In some cases, the medical device data screen 1660 may display the physiologic sensor data as discrete values in a numerical format (e.g., EtCO2 2250, SpO2 2252, NIBP 2254, and HR 2256) and/or in a graphical format (e.g., EtCO2 2260, SpO2 2261, lung resistance 2262, lung elastance 2263, NIBP 2264, and HR 2265).

In an implementation, the display of the physiologic data may include one or more event markers (e.g., the markers 2290, 2292, 2294, and 2296) overlaid on the physiologic data and/or in another graphical or tabular format. In an implementation, caregivers may manually input event marker data at the companion device 105 and on the data screen 1660 that correspond to certain types of medical interventions (e.g., administration of oxygen or medication). In some examples, upon selection of the medical device data screen 1660, the companion device 105 may transmit a data request, alone or as part of a bulk data transfer request, for event marker data to display on the data screen 1660. Upon selection of one of the event marker annotations 2290, 2292, 2294, and 2296 the companion device 105 may cause display of details about the selected event marker, such as amount of energy in an applied shock, amount of medication administered, display screen of defibrillator waveforms and/or ventilation data, and/or a snapshot view of data from the display interface at the medical/other device(s) 180 and/or the device view 1315 and/or 1316 at the time associated with the selected event marker

In an implementation, the medical device data screen 1660 may display the evaluations of the physiologic sensor data by the CSG algorithm 121. The data screen 1660 may provide these evaluations graphically. For example, ranges for the various indicators may be indicated as low, normal, or high with a graphical distinction as shown schematically with various cross-hatch patterns, colors, amount of fill of a graphic symbol, etc. In an implementation, the data screen 1660 may provide the evaluation of the physiologic signal with an evaluation indicator shown, for example, as an arrow 2279 positioned relative to the graphically provided ranges. Alternatively or additionally, the medical device data screen 1660 may provide textual indications of the evaluation of the physiologic signal (e.g., the evaluation indications 2270, 2271, 2272, 2273, 2274, and 2275).

Furthermore, in some embodiments, medical device data screen 1660 may provide a medical device status window 2280. The CSG algorithm 121 may receive medical device status information as a CSG input from medical/other devices. The CSG algorithm 121 may receive this information automatically according to a predetermined schedule, in response to a detected clinical event, and/or in response to a query or instruction from the CSG algorithm 121 to the medical/other device(s) 180.

Referring to FIG. 27B, an example of the mechanical ventilation apparatus for a ventilation system (such as a respiratory distress ventilator 4102) is illustrated. The mechanical ventilation apparatus 2740 may include a gas mover/gas flow generator 2741 configured to move oxygen from the oxygen source 2730 through an inspiratory circuit 2743 to the patient 101 via a patient interface 2744. The ventilation system 280 may use the gas-mover 2741 to provide a range of therapeutic ventilation modalities to provide various interventions such as those described with reference to FIG. 27.

In some embodiments, the ventilation system 280 may provide ventilation during CPR, and may, for example work in coupling and integrating with one or more critical care monitors, such as one or more patient monitor/defibrillator(s) 285, for example. In some embodiments, for example, during mask ventilation, unprotected airway, the ventilation system 280 may deliver 2 breaths for every 30 compressions as measured by a monitor. In some embodiments, for example, when the airway is protected (e.g., endotracheal tube, Combitube, etc.), the ventilation system 280 may continuously deliver 10 breaths/min independent of the compression rate (asynchronous).

FIG. 36 illustrates aspects of an example of the mechanical ventilation apparatus for a ventilation system such as a respiratory distress ventilator, including a controller 2750 and connected to an oxygen source 2730, used in providing mechanical ventilation to a patient. The patient interface 2744 may include an appropriate gas delivery device, such as an intubation tube, mask, nasal cannula, etc. The mechanical ventilation apparatus 2740 further includes an expiratory circuit 2745 and an exhalation valve 2748. Both the inspiratory circuit 2743 and the expiratory circuit 2745 include sensors 2747. The sensors 2747 may include, for example, but not limited to, the pneumotachometer 275, the airway pressure sensor 274, and the spirometer 276. The sensors 2747 enable the ventilation system 280 to measure the patient's respiratory efforts as well as the performance of the ventilation system 280 when providing mechanical respiratory assistance to the patient. The sensors 2747 may generate and provide data to the CSG processor module 120, the data including but not limited to flow rate, tidal volume and minute volume (Ve), respiratory mechanics (e.g., resistance and compliance) and spirometry, and may include, for example, forced vital capacity (FVC), forced vital capacity at 1 second (FEV1) and peak expiratory flow rate (PEF or PEFR). In addition, the patient monitor/defibrillator 285 or another medical device may provide, for example, capnography and/or oximetry data, such as oxyhemoglobin and carboxyhemoglobin saturation and mainstream or other capnographic data such as end tidal CO2 (EtCO2). This data may allow, for example, for calculation of CO2 elimination rate and volumetric capnography, which may include using flow data from the ventilation system 280.

In some embodiments, for patients who require supplemental oxygen (O2), the ventilation system 280 may provide a number of methods to support oxygenation by invasive and non-invasive ventilation. For example, one method may include using a small reservoir bag system that allows entrainment from an O2 flow source. In some embodiments, in a first method, the user may manage the O2 delivery to the patient while monitoring the oxygen saturation (SpO2) measured by the patient monitor/defibrillator 285 or another medical device. A second method may make use of an innovative smart O2 valve (SOV) or module, which may, for example, attach to the inspiratory circuit 2743, a high-pressure O2 cylinder/source and the patient monitor/defibrillator 285 or another medical device This may, for example, provide for automatic control of the patient's oxygenation using physiologic closed loop control (PCLC) where, the SpO2 signal may be used to regulate the output of the SOV. A third method may provide for the functional integration of a portable O2 concentrator (POC), which may, for example, use PCLC to regulate the O2 output of the POC and the O2 entrainment into the ventilation system 280.

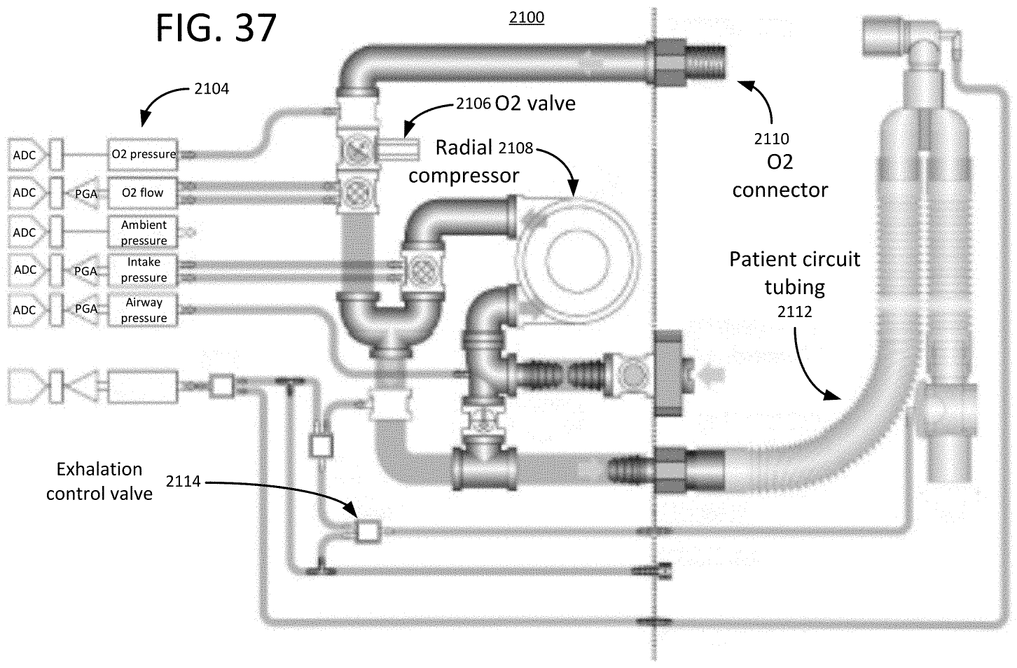

FIG. 37 illustrates aspects of an example pneumatic system 2100 that can be used with a respiratory distress ventilator, in accordance with embodiments of the present disclosure. As shown, the pneumatic system 2100 includes an oxygen connector 2110, patient circuit tubing 2112 that may include patent inhalation and exhalation circuit components, an exhalation control valve 2114, a radial compressor 2108 or other gas moving component such as a blower, an oxygen valve 2106, and aspects 2104 associated with control and/or measurement of oxygen pressure, oxygen flow, ambient pressure, intake pressure and patient airway pressure, which may include programmable-gain amplifiers (PGAs), analog-to-digital converters (ADCs), and other components

The oxygen valve 2106 and the compressor 2108 may provide the appropriate gas mixture for the patient during ventilation. The system 2100 may include transducers for pressure measurements including oxygen input supply and barometric pressure. The patient circuit tubing 2112 may include an inspiratory portion that provides gas to the patient, as well as an expiratory portion that exhausts gas directly to the atmosphere without return to the ventilator 2000. The ventilator 2000 pneumatically controls the exhalation control valve 2114. A transducer within the ventilator 2000 measures the airway pressure during ventilation.

In some embodiments, a coaxial patient circuit may be used, in which, while appearing as a single hose or circuit, the expiratory hose surrounds the inspiratory hose. At both the patient and device connections, a plenum divides the gas paths. In some embodiments, such a patient circuit configuration may provide benefits that may include reduced deadspace, reduced smallest usable footprint, minimized weight/pull on airway, and being single part based with no assembly or troubleshooting needed.

An external high pressure gas source connects to the ventilator 2000 using a high pressure oxygen input port. The source may be a medical grade oxygen system or oxygen cylinder supply, for example.

FIG. 38 illustrates aspects of an example external gas supply system 2200 that can be used with a respiratory distress ventilator 2208, in accordance with embodiments of the present disclosure. Depicted aspects include an oxygen tank 2202 or other oxygen output 2204 (such as a high pressure hose for use with oxygen supply 2206, and an oxygen tank 2210 shown coupled to the respiratory distress ventilator 2208.

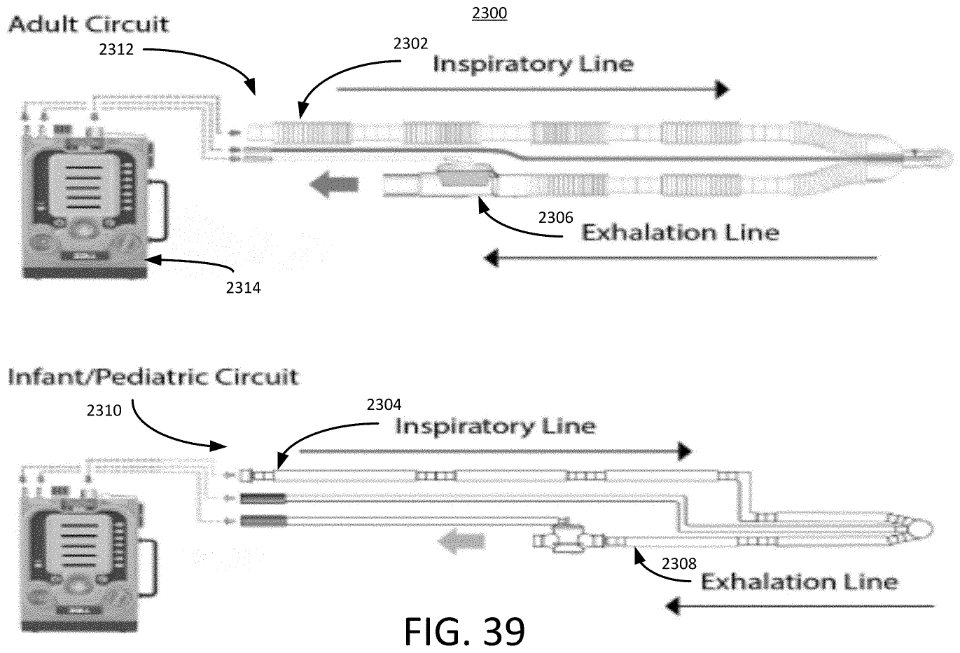

FIG. 39 illustrates aspects of example patient circuits 2300 that can be used with a respiratory distress ventilator 2314, in accordance with embodiments of the present disclosure. Depicted aspects include an adult circuit 2312, including an inspiratory line 2302 and an expiratory line 2306, and an infant/pediatric circuit 2310, including an inspiratory line 2304 and an expiratory line 2308.

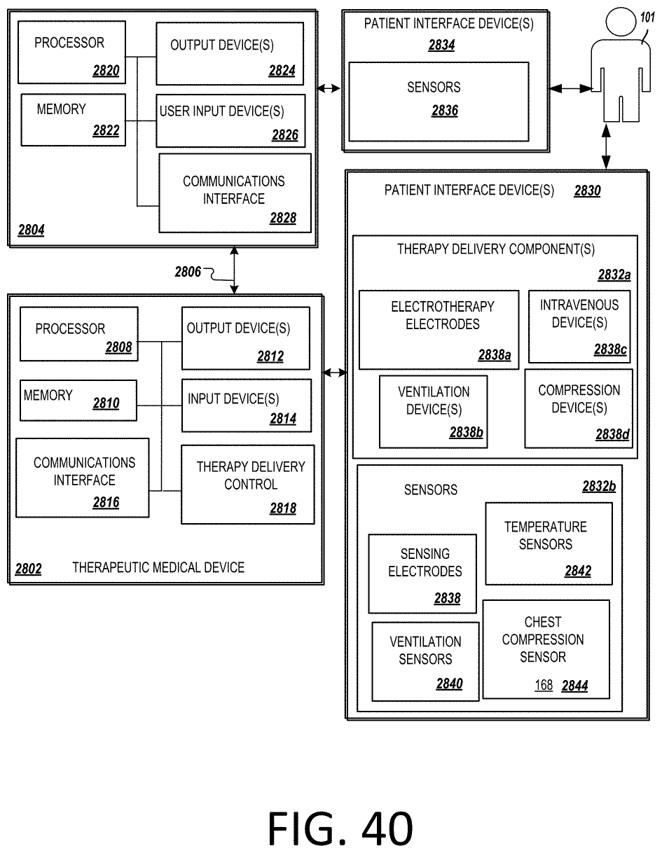

In FIG. 40, the components 2808, 2810, 2812, 2814, 2816, and 2818 are communicatively coupled (directly and/or indirectly) to each other for bi-directional communication. Similarly, the components 2820, 2822, 2824, 2826, and 2828 are communicatively coupled (directly and/or indirectly) to each other for bi-directional communication.

In some implementations, the components 2808, 2810, 2816, and/or 2818 of the therapeutic medical device 2802 may be combined into one or more discrete components and components 2816 and/or 2818 may be part of the processor 2808. The processor 2808 and the memory 2810 may include and/or be coupled to associated circuitry in order to perform the functions described herein. Additionally, the components 2820, 2822, and 2828 of companion device 2804 may be combined into one or more discrete components and component 2828 may be part of the processor 2820. The processor 2820 and the memory 2821 may include and/or be coupled to associated circuitry in order to perform the functions described herein.

In some implementations, the therapeutic medical device 2802 may include the therapy delivery control module 2818. For example, the therapy delivery control module 2818 may be an electrotherapy delivery circuit that includes one or more high-voltage capacitors configured to store electrical energy for a pacing pulse or a defibrillating pulse. The electrotherapy delivery circuit may further include resistors, additional capacitors, relays and/or switches, electrical bridges such as an H-bridge (e.g., including a plurality of insulated gate bipolar transistors or IGBTs), voltage measuring components, and/or current measuring components. As another example, the therapy delivery control module 2818 may be a compression device electro-mechanical controller configured to control a mechanical compression device. As a further example, the therapy delivery control module 2818 may be an electro-mechanical controller configured to control drug delivery, temperature management, ventilation, and/or other type of therapy delivery.

The therapeutic medical device 2802 may incorporate and/or be configured to couple to one or more patient interface devices 2830. The patient interface devices 2830 may include one or more therapy delivery component(s) 2832a and one or more sensor(s) 2832b. Similarly, the companion device 2804 may be adapted for medical use and may incorporate and/or be configured to couple to one or more patient interface device(s) 2834. The patient interface device(s) 2834 may include one or more sensors 2836. The sensor(s) 2836 may be substantially as described herein with regard to the sensor(s) 2832b.

The sensor(s) 2832b and 2836 may include sensing electrodes (e.g., the sensing electrodes 2838), ventilation and/or respiration sensors (e.g., the ventilation and/or respiration sensors 2830), temperature sensors (e.g., the temperature sensor 2842), chest compression sensors (e.g., the chest compression sensor 2844), etc. In some implementations, the information obtained from the sensors 2832b and 2836 can be used to generate information displayed at the therapeutic medical device 2802 and simultaneously at the display views at companion device 2804 and described above. In one example, the sensing electrodes 2838 may include cardiac sensing electrodes. The cardiac sensing electrodes may be conductive and/or capacitive electrodes configured to measure changes in a patient's electrophysiology to measure the patient's ECG information. The sensing electrodes 2838 may further measure the transthoracic impedance and/or a heart rate of the patient. The ventilation and/or respiration sensors 2830 may include spirometry sensors, flow sensors, pressure sensors, oxygen and/or carbon dioxide sensors such as, for example, one or more of pulse oximetry sensors, oxygenation sensors (e.g., muscle oxygenation/pH), O2 gas sensors and capnography sensors, impedance sensors, and combinations thereof. The temperature sensors 2842 may include an infrared thermometer, a contact thermometer, a remote thermometer, a liquid crystal thermometer, a thermocouple, a thermistor, etc. and may measure patient temperature internally and/or externally. The chest compression sensor 2844 may include one or more motion sensors including, for example, one or more accelerometers, one or more force sensors, one or more magnetic sensors, one or more velocity sensors, one or more displacement sensors, etc. The chest compression sensor 2844 may provide one or more signals indicative of the chest motion to the therapeutic medical device 2802 via a wired and/or wireless connection. The chest compression sensor 2844 may be, for example, but not limited to, a compression puck, a smart-phone, a hand-held device, a wearable device, etc. The chest compression sensor 2844 may be configured to detect chest motion imparted by a rescuer and/or an automated chest compression device (e.g., a belt system, a piston system, etc.). The chest compression sensor 2844 may provide signals indicative of chest compression data including displacement data, velocity data, release velocity data, acceleration data, force data, compression rate data, dwell time data, hold time data, blood flow data, blood pressure data, etc. In an implementation, the defibrillation and/or pacing electrodes may include or be configured to couple to the chest compression sensor 2844.

In various implementations, the sensors 2832b and 2836 may include one or more sensor devices configured to provide sensor data that includes, for example, but not limited to ECG, blood pressure, heart rate, respiration rate, heart sounds, lung sounds, respiration sounds, end tidal CO₂, saturation of muscle oxygen (SMO₂), oxygen saturation (e.g., SpO₂ and/or PaO₂), cerebral blood flow, point of care laboratory measurements (e.g., lactate, glucose, etc.), temperature, electroencephalogram (EEG) signals, brain oxygen level, tissue pH, tissue fluid levels, images and/or videos via ultrasound, laryngoscopy, and/or other medical imaging techniques, near-infrared spectroscopy, pneumography, cardiography, and/or patient movement. Images and/or videos may be two-dimensional or three-dimensional, such a various forms of ultrasound imaging.

The one or more therapy delivery components 2832a may include electrotherapy electrodes (e.g., the electrotherapy electrodes 2838a), ventilation device(s) (e.g., the ventilation devices 2838b), intravenous device(s) (e.g., the intravenous devices 2838c), compression device(s) (e.g., the compression devices 2838d), etc. For example, the electrotherapy electrodes 2838a may include defibrillation electrodes, pacing electrodes, and combinations thereof. The ventilation devices 2838b may include a tube, a mask, an abdominal and/or chest compressor (e.g., a belt, a cuirass, etc.), etc. and combinations thereof. The intravenous devices 2838c may include drug delivery devices, fluid delivery devices, and combinations thereof. The compression devices 2838d may include mechanical compression devices such as abdominal compressors, chest compressors, belts, pistons, and combinations thereof. In various implementation, the therapy delivery component(s) 2832a may be configured to provide sensor data and/or be coupled to and/or incorporate sensors. For example, the electrotherapy electrodes 2838a may provide sensor data such as transthoracic impedance, ECG, heart rate, etc. Further the electrotherapy electrodes 2838a may include and or be coupled to a chest compression sensor. As another example, the ventilation devices 2838b may be coupled to and/or incorporate flow sensors, gas species sensors (e.g., oxygen sensor, carbon dioxide sensor, etc.), etc. As a further example, the intravenous devices 2838c may be coupled to and/or incorporate temperature sensors, flow sensors, blood pressure sensors, etc. As yet another example, the compression devices 2838d may be coupled to and/or incorporate chest compression sensors, patient position sensors, etc. The therapy delivery control modules 2818 may be configured to couple to and control the therapy delivery component(s) 2832a, respectively.

The one or more sensor(s) 2832b and 2836 and/or the therapy delivery component(s) 2832a may provide sensor data. The patient data provided at the display screens of the therapeutic medical device 2802 and companion device 2804 may display the sensor data. For example, the therapeutic medical device 2802 may process signals received from the sensor(s) 2832b and/or the therapy delivery component(s) 2832a to determine the sensor data. Similarly, the companion device 2804 may process signals received from the sensor(s) 2836 and/or sensor data from the sensors 2832b received via the therapeutic medical device 2802 to determine the sensor data.

While certain embodiments have been described, these embodiments have been presented by way of example only and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1. An apparatus for treating a patient experiencing respiratory distress, the apparatus comprising:
a respiratory distress management device, comprising:
a housing;
a mechanical ventilation apparatus, disposed within the housing, for providing mechanical ventilation to the patient, comprising:
a gas flow generator disposed within the housing; and
a gas delivery apparatus, disposed at least partially within the housing, coupled with the gas flow generator, comprising a patient circuit extending from the housing and configured to interface with the patient at least in part for delivery of gas to the patient; and
a controller, disposed within the housing, the controller comprising a processor and a memory, the controller being configured to:
determine whether a fault mode condition exists at a particular time;
if the fault mode condition is determined not to exist, enable control of the mechanical ventilation apparatus of the respiratory distress management device by at least one source in delivering mechanical ventilation to the patient, via signals received by the controller from the at least one source; and
if the fault mode condition is determined to exist, control the mechanical ventilation apparatus of the respiratory distress management device in delivering the mechanical ventilation to the patient.

2. The apparatus of claim 1, wherein, if the fault mode condition is determined to exist at the particular time, the controller controls the mechanical ventilation apparatus of the respiratory distress management device in delivering the mechanical ventilation to the patient according to at least one of: previously stored ventilation control parameter values and previously stored last received valid ventilation control parameter values.

3. (canceled)

4. The apparatus of claim 1, wherein the controller is configured to:
determine whether the fault mode condition exists, wherein the fault mode condition is associated with the respiratory distress management device, and wherein the at least one source is separate from the respiratory distress management device.

5. The apparatus of claim 1, wherein the controller is configured to:
determine whether a fault mode condition exists at a particular time, wherein the particular time is a first predetermined time;
if the fault mode condition is determined not to exist at the first predetermined time, enable control of the mechanical ventilation apparatus of the respiratory distress management device by at least one source in delivering mechanical ventilation to the patient, via signals received by the controller from the at least one source; and
if the fault mode condition is determined to exist at the first predetermined time, control the mechanical ventilation apparatus of the respiratory distress management device in delivering the mechanical ventilation to the patient.

6. The apparatus of claim 5, wherein the controller is configured to:
determine whether a fault mode condition exists at a second predetermined time, wherein the second predetermined time follows the first predetermined time by a predetermined period of time;
if the fault mode condition is determined not to exist at the second predetermined time, enable control of the mechanical ventilation apparatus of the respiratory distress management device by at least one source in delivering mechanical ventilation to the patient, via signals received by the controller from the at least one source; and
if the fault mode condition is determined to exist at the second predetermined time, control the mechanical ventilation apparatus of the respiratory distress management device in delivering the mechanical ventilation to the patient.

7. (canceled)

8. The apparatus of claim 6, wherein the controller is configured such that the predetermined period of time is set to one of: one second, between 0.1 second and 1 second, and between 1 second and 10 seconds.

9-11. (canceled)

12. The apparatus of claim 1, wherein the mechanical ventilation apparatus comprises:
at least one pressure sensor, disposed within the mechanical ventilation apparatus, configured to sense signals representative of gas flow within the gas delivery apparatus; and
wherein the controller is configured to:
receive the signals representative of the gas flow;
based at least in part on the received signals representative of the gas flow, generate respiratory parameter data corresponding with at least one respiratory parameter of the patient; and
transmit the respiratory parameter data to a second device coupled with the respiratory distress management device, the respiratory parameter data being for use in determining a respiratory status of the patient.

13. The apparatus of claim 12, wherein the controller is configured to transmit the respiratory parameter data to the second device for use by the second device in displaying at least one of data related to the respiratory status of the patient on a graphical user interface of a display of the second device and user-interactive context sensitive guidance relating to treatment of the patient.

14. The apparatus of claim 13, wherein the controller is configured to transmit the respiratory parameter data to the second device for use by the second device in displaying user-interactive context sensitive guidance relating to treatment of the patient, the context sensitive guidance comprising a therapy recommendation provided to a user of the second device and relating to a therapy to be delivered to the patient at least in part by the respiratory distress management device upon selection by the user of the therapy recommendation.

15-16. (canceled)

17. The apparatus of claim 12, wherein the controller is configured to generate respiratory parameter data corresponding with at least one respiratory parameter of the patient, wherein the at least one respiratory parameter comprises forced vital capacity (FVC) and forced expiratory volume (FEV).

18-19. (canceled)

20. The apparatus of claim 12, wherein the mechanical ventilation apparatus comprises at least one spirometer, and wherein the at least one pressure sensor is coupled with or part of at least one pneumotachometer disposed within the mechanical ventilation apparatus.

21-22. (canceled)

23. The apparatus of claim 1, wherein the controller determines whether the fault mode condition exists using one or more algorithms stored in the memory of the controller and executed by the processor of the controller.

24. The apparatus of claim 1, wherein the controller is configured to enable the control by the at least one source, wherein the at least one source comprises at least one of: a portable computing device, a tablet, a critical care monitor, a defibrillator, and one or more aspects of a cloud-based computing system.

25-28. (canceled)

29. The apparatus of claim 1, wherein the controller is configured to enable the control by the at least one source, wherein the control by the at least one source comprises utilizing closed loop ventilation control based on oxygen concentration measurements of the patient's blood, wherein the closed loop ventilation control comprises utilizing at least one oxygen saturation sensor to monitor an oxygen concentration of the patient's blood and adjusting oxygen delivery during the delivery of the mechanical ventilation to the patient to maintain the oxygen concentration of the patient's blood at a desired level or range.

30. The apparatus of claim 29, wherein the controller is configured to enable control by the at least one source, wherein the at least one source comprises a critical care monitor, and wherein the at least one oxygen saturation sensor is part of a pulse oximeter of the critical care monitor.

31. The apparatus of claim 1, wherein the controller is configured to, in an event that the fault mode condition is determined to exist, switch from enabling the control of the mechanical ventilation by the at least one source to the controller controlling the mechanical ventilation.

32. The apparatus of claim 1, wherein the fault mode condition indicates that the control by the at least one source would be at least one of: insufficiently safe,. insufficiently reliable with respect to receipt of mechanical ventilation control data by the controller from the at least one source, and suboptimal with respect to the treatment of the patient relative to the control by the controller.

33-35. (canceled)

36. The apparatus of claim 1, wherein the fault mode condition indicates that ventilation control data received by the controller from the at least one source is at least one of: invalid, corrupt, and inconsistent.

37-38. (canceled)

39. The apparatus of claim 1, wherein the fault mode condition indicates that enabling the control by the at least one source would cause operation of the mechanical ventilation apparatus according to at least one ventilation parameter value that exceeds at least one range comprising at least one of: a range associated with patient safety, a flow rate range, and a pressure range.

40-42. (canceled)

43. The apparatus of claim 1, wherein the fault mode condition indicates that enabling the control by the at least one source would cause operation of the mechanical ventilation apparatus according to values for at least two parameters, wherein a combination of the values would exceed a range of allowed value combinations for the at least two parameters.

44-46. (canceled)

47. The apparatus of claim 1, wherein the respiratory distress management device is configured such that, upon a reset of the controller following a controller processing freeze that occurred during the control by the at least one source, if communication ability does not exist between the respiratory distress management device and the at least one source, the controller controls the mechanical ventilation in accordance with at least one of: one or more backup ventilation control parameter values stored in the memory of the controller, and current ventilation control data from the at least one source.

48. The apparatus of claim 1, wherein the controller is configured to perform cyclic redundancy checks on mechanical ventilation control data received from the at least one source.

49. (canceled)

50. The apparatus of claim 1, wherein the controller is configured to perform processing utilizing at least one of: task prioritization, data sampling protection, one or more histograms in data handling in order to minimize data noise, a single thread configuration, thread-safe operating system objects, a round robin software processing design in order to minimize processing delay, performance of token count checks on ventilation control data received from the at least one source, disabling of software processing interrupts under one or more conditions, prioritization with respect to one or more aspects of communication with the at least one source, one or more checks relating to a ventilation control data receipt rate from the at least one source, maintainance of a minimum portion of unused memory in order to protect against potential data storage overload, an acknowledgement (ACK) and non-acknowledgement (NACK) scheme in ensuring receipt of ventilation control data from the at least one source, an automatic reset in the event of a software processing freeze or deadlock condition, continuous communication between the respiratory distress management device and the at least one source in correcting any data receipt errors continuously, and one or more checks to ensure that the at least one source is properly configured for communication with the respiratory distress management device.

51-63. (canceled)

64. The apparatus of claim 1, wherein the controller is configured to generate user alarms relating to particular detected conditions, the alarms comprising at least one of: alarms to provide alerts regarding potentially unsafe ventilation conditions, alarms to provide alerts regarding potentially unsafe respiratory parameter values of the patient, alarm indications that are for display to a user of the respiratory distress management system on a graphical user interface that is separate from the respiratory distress management system, alarms relating to ventilation parameter values relating to control by the at least one source, alarms relating to patient physiological parameters, one or more alarms relating to a high breath rate of the patient, and one or more alarms relating to an incomplete exhalation condition of the patient.

65-72. (canceled)

73. A system for treating a patient experiencing respiratory distress, the system comprising:
a respiratory distress management system, comprising:
a mechanical ventilation system for providing mechanical ventilation to the patient, comprising at least one pressure sensor configured to sense signals representative of gas flow within the mechanical ventilation system; and
a controller, comprising a processor and a memory, the controller being configured to:
determine whether a fault mode condition exists at a particular time;
if the fault mode condition is determined not to exist, enable control of the mechanical ventilation system of the respiratory distress management system by at least one source in delivering mechanical ventilation to the patient, via signals received by the controller from the at least one source; and
if the fault mode condition is determined to exist, control the mechanical ventilation system of the respiratory distress management system in delivering the mechanical ventilation to the patient.

74. The system of claim 73, wherein the controller is configured to:
determine whether the fault mode condition exists, wherein the fault mode condition is associated with the respiratory distress management system, and wherein the at least one source is separate from the respiratory distress management system.

75. The system of claim 73, wherein the controller is configured to:
receive the signals representative of the gas flow;
based at least in part on the received signals representative of the gas flow, generate respiratory parameter data corresponding with at least one respiratory parameter of the patient; and
transmit the respiratory parameter data to a device coupled with the respiratory distress management system, the respiratory parameter data being for use in determining a respiratory status of the patient.

76. The system of claim 75, wherein the controller is configured to transmit the respiratory parameter data to the device for use by the device in displaying data related to the respiratory status of the patient on a graphical user interface of a display of the device.

77. The system of claim 76, wherein the controller is configured to transmit the respiratory parameter data to the device for use by the device in displaying user-interactive context sensitive guidance relating to treatment of the patient.

78. The system of claim 77, wherein the controller is configured to transmit the respiratory parameter data to the device for use by the device in displaying user-interactive context sensitive guidance relating to treatment of the patient, the context sensitive guidance comprising a therapy recommendation provided to a user of the device and relating to a therapy to be delivered at least in part by the respiratory distress management device upon selection by the user of the therapy recommendation.

79-82. (canceled)

83. The system of claim 73, wherein the controller determines whether the fault mode condition exists using one or more algorithms stored in the memory of the controller and executed by the processor of the controller.

84-87. (canceled)

88. The system of claim 73, wherein the controller is configured to enable the control by the at least one source, wherein the control by the at least one source comprises utilizing closed loop ventilation control based on oxygen concentration measurements of the patient's blood, wherein the closed loop ventilation control comprises utilizing at least one oxygen saturation sensor to monitor an oxygen concentration of the patient's blood and adjusting oxygen delivery during the delivery of the mechanical ventilation to the patient to maintain the oxygen concentration of the patient's blood at a desired level or range.

89-130. (canceled)

131. A computer-implemented method for treating a patient experiencing respiratory distress, the method comprising:
a controller, disposed within a respiratory distress management device, the controller comprising a processor and a memory, determining whether, at a particular time, a fault mode condition exists, the fault mode condition being associated with the respiratory distress management device and at least one source that is separate from the respiratory distress management device;
if the fault mode condition is determined not to exist, the controller enabling control of a mechanical ventilation system of the respiratory distress management device by the at least one source in delivering mechanical ventilation to the patient, via signals received by the controller from the at least one source; and
if the fault mode condition is determined to exist, the controller controlling the mechanical ventilation system of the respiratory distress management device in delivering the mechanical ventilation to the patient.

132. The method of claim 131, comprising the controller determining whether the fault mode condition exists, wherein the mechanical ventilation system comprises:
at least one pressure sensor, disposed within a gas delivery system of the mechanical ventilation system, configured to sense signals representative of gas flow within the gas delivery system; and
wherein the controller:
receives the signals representative of the gas flow;
based at least in part on the received signals representative of the gas flow, generates respiratory parameter data corresponding with at least one respiratory parameter of the patient; and
transmits the respiratory parameter data to a second device coupled with the respiratory distress management device, the respiratory parameter data being for use in determining a respiratory status of the patient.

133-143. (canceled)

144. The method of claim 131, wherein the controller enables the control by the at least one source, wherein the control by the at least one source comprises utilizing closed loop ventilation control based on oxygen concentration measurements of the patient's blood, wherein the closed loop ventilation control comprises utilizing at least one oxygen saturation sensor to monitor an oxygen concentration of the patient's blood and adjusting oxygen delivery during the delivery of the mechanical ventilation to the patient to maintain the oxygen concentration of the patient's blood at a desired level or range.

145-342. (canceled)
